(19) United States
(12) Patent Application Publication
(10) Pub. No.: US 2016/0200789 A1
(43) Pub. Date: Jul. 14, 2016

(54) IL4 CONJUGATED TO ANTIBODIES AGAINST EXTRACELLULAR MATRIX COMPONENTS

(71) Applicant: PHILOGEN S.P.A., Sienna (IT)

(72) Inventors: Teresa Hemmerle, Vaduz (LI); Dario Neri, Buchs (CH)

(73) Assignee: Philogen S.P.A., Sienna (IT)

(21) Appl. No.: 14/787,140

(22) PCT Filed: Feb. 28, 2014
(86) PCT No.: PCT/EP2014/053998

§ 371 Date: Oct. 26, 2015

(30) Foreign Application Priority Data
Apr. 26, 2013 (GB) 1307599.9
Oct. 11, 2013 (GB) 1318043.5
Nov. 22, 2013 (GB) 1320647.9

(51) Int. Cl. C07K 14/54; A61K 31/573; A61K 39/395; C07K 16/18; A61K 38/20

(52) U.S. Cl. CPC C07K 14/5406; C07K 16/18; A61K 38/2026; A61K 39/3955; A61K 31/573; C07K 2317/626; C07K 2317/565; C07K 2319/035; C07K 2319/30; C07K 2317/21; C07K 2317/76; A61K 2039/505

(57) ABSTRACT

A conjugate comprising interleukin-4 (IL4) and a specific binding member is disclosed. The specific binding member preferably binds an extra-cellular matrix component associated with neoplastic growth and/or angiogenesis, and the conjugate may be used for targeting IL4 to tissues in vivo. In particular, the therapeutic use of such conjugates in the treatment of a disease/disorder, such as cancer and/or autoimmune diseases, including rheumatoid arthritis (RA), multiple sclerosis (MS), endometriosis, inflammatory bowel disease (IBD), psoriasis, psoriatic arthritis, and periodontitis is envisaged. Other diseases which may be treated or prevented using the conjugates include autoimmune insulitis and diabetes, in particular autoimmune diabetes. In the treatment of cancer, the conjugate may be administered in combination with a conjugate comprising either interleukin-12 (IL12) or interleukin-2 (IL2) and a specific binding member. In the treatment of autoimmune diseases, the conjugate may be administered in combination with a glucocorticoid, such as dexamethasone.

### FIELD OF THE INVENTION

The present invention relates to a conjugate comprising interleukin-4 (IL4) and a specific binding member. The specific binding member preferably binds an extra-cellular matrix component associated with neoplastic growth and/or angiogenesis, and the conjugate may be used for targeting IL4 to tissues in vivo. In particular, the present invention relates to the therapeutic use of such conjugates in the treatment of a disease/disorder, such as cancer and/or autoimmune diseases, including rheumatoid arthritis (RA), multiple sclerosis (MS), endometriosis, inflammatory bowel disease (IBD), psoriasis, psoriatic arthritis, and periodontitis. Other diseases which may be treated or prevented using the conjugates of the invention include autoimmune insulitis and diabetes, in particular autoimmune diabetes. In the treatment of cancer, the conjugate may be administered in combination with a conjugate comprising either interleukin-12 (IL12) or interleukin-2 (IL2) and a specific binding member. In the treatment of autoimmune diseases, the conjugate may be administered in combination with a glucocorticoid, such as dexamethasone.

### BACKGROUND TO THE INVENTION

Cytokines are key mediators of innate and adaptive immunity. Many cytokines have been used for therapeutic purposes in patients, such as those with advanced cancer, but their administration is typically associated with severe toxicity, hampering dose escalation to therapeutically active regimens and their development as anticancer drugs, for example. To overcome these problems, the use of ‘immunocytokines’ (i.e. cytokines fused to antibodies or antibody fragments) has been proposed, with the aim to concentrate the immune-system stimulating activity at the site of disease while sparing normal tissues (Savage et al., 1993; Schrama et al., 2006; Neri et al. 2005; Dela Cruz et al., 2004; Reisfeld et al., 1997; Konterman et al., 2012).

For example, several pro-inflammatory immunocytokines (e.g., those based on IL2, IL12, IL15, TNF) have been shown to display a potent anti-tumoural effect in mouse models of cancer (Borsi et al. 2003; Carnemolla et al., 2002; Frey et al., 2010; Kaspar et al., 2007; Pasche et al., 2012). In contrast, anti-inflammatory immunocytokines (e.g., those based on IL10) have been shown to be capable of conferring a therapeutic benefit in mouse models of chronic inflammatory conditions (rheumatoid arthritis, endometriosis [Schwager et al. 2011; Schwager et al., 2009]) but have no impact on tumour growth.

Antibodies specific to splice-isoforms of fibronectin and of tenascin-C have been described as vehicles for pharmacodelivery applications, as these antigens are virtually undetectable in the normal healthy adult (with the exception of the placenta, endometrium and some vessels in the ovaries) while being strongly expressed in the majority of solid tumours and lymphomas, as well as other diseases (Brack et al., 2006; Pedretti et al., 2009; Schliemann et al. 2009). For example, antibodies F8 and L19, specific to the alternatively-spliced EDA and EDB domains of fibronectin, respectively, and anti-tenascin C antibody F16 (Brack et al. 2006, Villa et al., 2008, Viti et al., 1999), have been employed for the development of armed antibodies, some of which have begun clinical testing in oncology and in rheumatology (Eigentler et al., 2011; Papadia et al., 2012). The tumour targeting properties of these antibodies have also been documented in mouse models of cancer and in patients.

Interleukin 4 (IL4) is a 14 kDa compact globular cytokine, stabilized by three internal disulfide bonds. It was first identified in the early 1980s as a B cell activating factor and exhibits many biological and immunoregulatory functions. It can control proliferation, differentiation and apoptosis in several cell types of hematopoietic and non-hematopoietic origin, including myeloid, mast, dendritic, endothelial, muscular and neuronal cells (Janeway, Immunobiology, 2005; Zamorano et al., 1996). As a key regulator in humoral and adaptive immunity, IL4 acts as a growth and survival factor for lymphocytes, stimulating the proliferation of activated B cells and T cells. The cytokine is crucially involved in the balance between Th1 and Th2 immunological responses, inducing the differentiation of naïve helper T cells into Th2 cells after antigen challenge (Janeway, Immunobiology, 2005). This activity is in stark contrast to the activity of IL12, which drives a Th1 polarization of immune response. Interleukin 4 also stimulates the proliferation of NK (natural killer) cells and up-regulates MHC class II production, therefore enhancing the antigen presentation (Chomarat et al., 1997).

Nowadays, IL4 is mostly considered to be an anti-inflammatory cytokine. However, although IL4 has been shown to exhibit disease-suppressing effects in in vivo mouse models of collagen-induced arthritis when high doses of murine IL4 were administered (Joosten et al., 1999), administration of low doses of murine IL4 showed no effect on the course of arthritis in the same mouse model (Joosten et al., 1999; Joosten et al., 1997). In contrast, administration of even low doses of murine IL10 in this mouse model lead to suppression of arthritis (Joosten et al., 1997). In addition, IL4 certainly does not exhibit anti-inflammatory properties under all conditions. For example, IL4 treatment has been shown to significantly accelerate the development of colitis in a mouse model of the disease (Fort et al., 2001). IL10 therefore represents a more promising candidate than IL4 for the preparation of immunoconjugates, in particular for the treatment of inflammatory conditions, such as RA and colitis.

The effect of IL4 on tumours is also far from clear. It appears that both expression patterns and doses influence the effect of IL4 on tumour growth. For example, opposite biological effects on endothelial cell migration have been observed at low (promotion) and high concentrations (inhibition) of IL4 (Volpert et al., 1998), while Li et al. (2008) reports that endogenous IL4 promotes tumour development by increasing tumour cell resistance to apoptosis while exogenous IL4 has anti-tumour effects. Fukushi et al. (2000) similarly discusses the variable effects of IL4 on angiogenesis reported in the literature, with the authors themselves concluding that IL4 induces angiogenesis both in vitro and in vivo in a corneal pocket assay. The paradoxical effect of IL4 on tumours is summarized, for example, in Li et al. (2009).

In addition, although preclinical studies with recombinant, untargeted, murine IL4 as therapeutic agent showed promising anti-tumour activity in various mouse models of cancer (Tepper et al., 1989; Tepper et al., 1992; Wei et al., 1995; Yu et al., 1993), which led to the clinical investigation of recombinant human IL4 in several cancer types (Wiernik et al., 2010; Whitehead et al., 2002), only minimal anti-tumour activity was observed in several clinical studies with more than 154 patients. Only one complete response in a patient with disseminated malignant melanoma and one in a patient with relapsed and resistant NHL was observed. Furthermore, IL4 therapy had substantial toxicity, the most common side effects being nausea, vomiting, diarrhea, headache/pain or malaise/fatigue/lethargy, including cases of grade 4 toxicities. As a consequence, the systemic use of IL4 was determined not to be suitable for cancer treatment (Whitehead et al., 2002; Whitehead et al., 1998; Kurtz et al., 2007).

Cytokines can be conjugated to antibody molecules to produce immunocytokines as mentioned above. However, although several immunocytokines have been successfully made, not all immunocytokines exhibit therapeutic effects, even where such effects would be expected based on the effects of treatment with the untargeted cytokine. For example, F8-IL7, F8-IL17, F8-IFN-alpha and IFN-gamma did not display the expected therapeutic effects or pharmaceutical quality when tested in mice (Pasche et al., 2011; Pasche et al., 2012; Frey et al., 2011; Ebbinghaus et al., 2005). In addition, not all immunocytokines retain the in vivo targeting properties of the parental antibody (Pasche & Neri 2012). Even in instances where the targeting properties of the parental antibody are retained, and the immunocytokine localizes efficiently to the site of disease, the therapeutic effect of the immunocytokine may be no better than that of the untargeted immunocytokine (Frey et al., 2011). The preparation of immunocytokines with therapeutic effects, such as anti-tumoural or anti-inflammatory activity, in particular the preparation of immunocytokines with more potent therapeutic activity than the untargeted cytokine, is therefore far from straightforward.

The preparation of a conjugate comprising IL4 fused to the Fc region of murine IgG2a is described in Walz et al. (2002). The authors postulate that fusion of the Fc region to IL4 will result in an increase the half-life of the conjugate compared with monomeric IL4, although no evidence for this is provided. The purpose of the Fc region in this instance is therefore not to target the IL4 to regions of disease as is the case with immunocytokines as described in the preceding paragraph.

The possibility of conjugating IL4 to targeting moieties is mentioned in WO03/092737 and WO01/10912. However, a conjugate comprising IL4 and a specific binding member which binds an extra-cellular matrix component associated with neoplastic growth and/or angiogenesis is not disclosed in either of these documents, nor is any therapeutic effect for such a conjugate demonstrated.

### STATEMENTS OF INVENTION

The present inventors have shown that interleukin-4 (IL4) can be conjugated to antibodies which bind an extra-cellular matrix component associated with neoplastic growth and/or angiogenesis, while retaining not only the targeting properties of the unconjugated antibody but also the biological activity of IL4. Furthermore, the present inventors have shown that these conjugates exhibit superior activity to untargeted IL4 in several disease models. As explained above, the preparation of such immunoconjugates remains difficult and unpredictable and there is thus no guarantee of success. Furthermore, given what was known about IL4 in the art, in particular in relation to the treatment of cancer, as well as inflammatory diseases, there was no incentive to prepare immunoconjugates comprising IL4. Even more surprisingly, immunoconjugates comprising IL4 show therapeutic activity in cancer. RA, psoriasis, endometriosis. MS and diabetes mellitus type 1. To our knowledge this is the first report of an immunocytokine displaying both anti-tumoural and anti-inflammatory activity.

In one aspect, the present invention therefore relates to a conjugate comprising interleukin-4 (IL4) and a specific binding member. For example, the conjugate of the present invention may consist of interleukin-4 (IL4) conjugated to a specific binding member. The specific binding member preferably binds an extra-cellular matrix component associated with neoplastic growth, and/or angiogenesis, and/or tissue remodeling. Most preferably the specific binding member binds an extra-cellular matrix component associated with neoplastic growth and/or angiogenesis.

The specific binding member is preferably an antibody. The specific binding member may comprise or consist of a single chain Fv (ScFv) or be a diabody. Most preferably, the specific binding member is a diabody.

Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site: antigen binding sites are formed by the association of the first domain of one polypeptide within the multimer with the second domain of another polypeptide within the multimer (WO94/13804; Holliger and Winter, Cancer Immunol. Immunother. (1997) 45:128-130; Holliger et al., Proc. Natl. Acad. Sci. USA 90 6444-6448, 1993).

In a diabody a heavy chain variable domain (VH) is connected to a light chain variable domain (VL) on the same polypeptide chain. The VH and VL domains are connected by a peptide linker that is too short to allow pairing between the two domains (generally around 5 amino acids). This forces paring with the complementary VH and VL domains of another chain. Examples of this format are shown in SEQ ID NOs 20, 59, 60 and 62. The VH and VL domains in a diabody are thus preferably linked by a 5 amino acid linker. The linker preferably has the sequence shown in SEQ ID NO: 23. The linker may consist of 3, 4, 5 or 6 amino acids.

Alternatively, a diabody for use in the invention may be a single chain diabody. In a single chain diabody two sets of VH and VL domains are connected together in sequence on the same polypeptide chain. For example, the two sets of VH and VL domains may be assembled in a single chain sequence as follows: (VH-VL)-(VH-VL), where the brackets indicate a set. In the single chain diabody format each of the VH and VL domains within a set is connected by a short or ‘non-flexible’ peptide linker. This type of peptide linker sequence is not long enough to allow pairing of the VH and VL domains within the set.

Generally a short or ‘non flexible’ peptide linker is around 5 amino acids. The two sets of VH and VL domains are connected as a single chain by a long or ‘flexible’ peptide linker. This type of peptide linker sequence is long enough to allow pairing of the VH and VL domains of the first set with the complementary VH and VL domains of the second set. Generally a long or ‘flexible’ linker is around 15 amino acids. Single chain diabodies have been previously generated (Kontermann, R. E., and Muller, R. (1999), J. Immunol. Methods 226: 179-188). A bispecific single chain diabody has been used to target adenovirus to endothelial cells (Nettelbeck et al., Molecular Therapy (2001) 3, 882-891).

The specific binding member preferably binds an extra-cellular matrix (ECM) component associated with neoplastic growth and/or angiogenesis, as mentioned above. The specific binding member may bind fibronectin. For example, the specific binding member may bind the Extra Domain-A (ED-A) isoform or Extra Domain-B (ED-B) isoform of fibronectin, or tenascin C. Preferably, the specific binding member binds the ED-A or ED-B of fibronectin, or binds the A1 domain of tenascin C. Most preferably, the specific binding member binds the ED-A of fibronectin.

The specific binding member may comprise an antigen binding site having the complementarity determining regions (CDRs), or the VH and/or VL domains of an antibody capable of specifically binding to an antigen of interest, for example, one or more CDRs or VH and/or VL domains of an antibody capable of specifically binding to an antigen of the ECM. Such antigens include fibronectin and tenascin C, as described above.

Thus, the specific binding member may comprise an antigen binding site of the antibody F8, the antibody L19 or the antibody F16, which have all been shown to bind specifically to ECM antigens. The specific binding member may comprise an antigen binding site having one, two, three, four, five or six CDRs, or the VH and/or VL domains of antibody F8, L19 or F16. The specific binding member may comprise or consist of the sequence of antibody F8, L19 or F16, in scFv or diabody format. Preferably, the specific binding member is a diabody.

F8, as referred to herein, is a human monoclonal diabody to the alternatively spliced ED-A domain of fibronectin. The sequence of this antibody is shown in SEQ ID NO: 20. An scFv version of this antibody is described Villa A et al. Int. J. Cancer. 2008 Jun. 1; 122(11): 2405-13. L19 is a human monoclonal scFv specific to the alternatively spliced ED-B domain of fibronectin and has been previously described (WO2006/119897). The sequence of this antibody is shown in SEQ ID NO: 33. The sequence of diabody versions of this antibody is shown in SEQ ID NOs. 59 and 62. F16 is a human monoclonal scFv specific to the A1 domain of Tenascin C and has been previously described (WO2006/050834). The sequence of this antibody is shown in SEQ ID NO: 42. The sequence of a diabody version of this antibody is shown in SEQ ID NO: 60.

An antigen binding site may comprise one, two, three, four, five or six CDRs of antibody F8. Amino acid sequences of the CDRs of F8 are:
SEQ ID NO:12 (CDR1 VH);
SEQ ID NO:13 (CDR2 VH);
SEQ ID NO:14 (CDR3 VH);
SEQ ID NO:15 (CDR1 VL);
SEQ ID NO:16 (CDR2 VL), and/or
SEQ ID NO:17 (CDR3 VL).

SEQ ID NOs 12-14 are the amino acid sequences of the VH CDR regions (1-3, respectively) of the human monoclonal antibody F8. SEQ ID NOs 15-17 are the amino acid sequences of the VL CDR regions (1-3, respectively) of the human monoclonal antibody F8. The CDRs of F8 shown in SEQ ID NOs 12-17 are encoded by the nucleotide sequences shown in SEQ ID NOs 1-6, respectively. The amino acid sequences of the VH and VL domains of F8 correspond to SEQ ID NO: 18 and SEQ ID NO: 19, respectively. The nucleotide sequences encoding the VH and VL domains of F8 correspond to SEQ ID NO: 7 and SEQ ID NO: 8, respectively. The sequence of the F8 diabody is shown in SEQ ID NO: 20.

An antigen binding site may comprise one, two, three, four, five or six CDRs of antibody L19. Amino acid sequences of the CDRs of L19 are:
SEQ ID NO:25 (CDR1 VH);
SEQ ID NO:26 (CDR2 VH);
SEQ ID NO:27 (CDR3 VH);
SEQ ID NO:28 (CDR1 VL);
SEQ ID NO:29 (CDR2 VL), and/or
SEQ ID NO:30 (CDR3 VL).

SEQ ID NOs 25-27 are the amino acid sequences of the VH CDR regions (1-3, respectively) of the human monoclonal antibody L19. SEQ ID NOs 28-30 are the amino acid sequences of the VL CDR regions (1-3, respectively) of the human monoclonal antibody L19. The amino acid sequence of the VH and VL domains of antibody L19 correspond to SEQ ID NO: 31 and SEQ ID NO: 32, respectively. The amino acid sequence of the scFv(L19) is given in SEQ ID NO: 33. The amino acid sequence of the L19 diabody is given in SEQ ID NO: 59. The amino acid sequence of the L19 diabody with an alternative VH/VL linker sequence to that of SEQ ID NO: 59 is given in SEQ ID NO: 62.

An antigen binding site may comprise one, two, three, four, five or six CDRs of antibody F16. Amino acid sequences of the CDRs of F16 are:
SEQ ID NO:34 (CDR1 VH);
SEQ ID NO:35 (CDR2 VH);
SEQ ID NO:36 (CDR3 VH);
SEQ ID NO:37 (CDR1 VL);
SEQ ID NO:38 (CDR2 VL), and/or
SEQ ID NO:39 (CDR3 VL).

SEQ ID NOs 34-36 are the amino acid sequences of the VH CDR regions (1-3, respectively) of the human monoclonal antibody F16. SEQ ID NOs 37-39 are to the amino acid of the VL CDR regions (1-3, respectively) of the human monoclonal antibody F16. The amino acid sequence of the VH and VL domains of antibody F16 correspond to SEQ ID NO: 40 and SEQ ID NO: 41, respectively. The amino acid sequence of the scFv(F16) is given in SEQ ID NO: 42. The amino acid sequence of the F16 diabody is given in SEQ ID NO: 60.

A specific binding member may comprise a VH domain having at least 70%, more preferably one of at least 75%, 80%, 85%, 90%, 95%, 96%, 97%, 98%, 99% or 100%, sequence identity to the F8 VH domain amino acid sequence of SEQ ID NO: 18, the L19 VH domain amino acid sequence of SEQ ID NO: 31, or the F16 VH domain amino acid sequence of SEQ ID NO: 40. The VH domain may be encoded by a nucleotide sequence having at least 70%, more preferably one of at least 75%, 80%, 85%, 90%, 95%, 96%, 97%, 98%, 99% or 100%, sequence identity to the F8 VH domain nucleotide sequence set forth in SEQ ID NO: 7.

A specific binding member may comprise have a VL domain having at least 70%, more preferably one of at least 75%, 80%, 85%, 90%, 95%, 96%, 97%, 98%, 99% or 100%, sequence identity to the F8 VL domain amino acid sequence of SEQ ID NO: 19, the L19 VL domain amino acid sequence of SEQ ID NO: 32, or the F16 VL domain amino acid sequence of SEQ ID NO: 41. The VL domain may be encoded by a nucleotide sequence having at least 70%, more preferably one of at least 75%, 80%, 85%, 90%, 95%, 96%, 97%, 98%, 99% or 100%, sequence identity to the F8 VL domain nucleotide sequence set forth in SEQ ID NO: 8.

Sequence identity is commonly defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), or the TBLASTN program, of Altschul et al. (1990) supra, generally employing default parameters. In particular, the psi-Blast algorithm (Nucl. Acids Res. (1997) 25 3389-3402) may be used.

Variants of these VH and VL domains and CDRs may also be employed in specific binding members for use in the conjugates described herein. Suitable variants can be obtained by means of methods of sequence alteration, or mutation, and screening. Particular variants for use as described herein may include one or more amino acid sequence alterations (addition, deletion, substitution and/or insertion of an amino acid residue), maybe less than about 20 alterations, less than about 15 alterations, less than about 10 alterations or less than about 5 alterations, 4, 3, 2 or 1. Alterations may be made in one or more framework regions and/or one or more CDRs. In particular, alterations may be made in VH CDR1, VH, CDR2 and/or VH CDR3.

The amino acid sequence of the F8 diabody is found in SEQ ID NO: 20. The F8 diabody may comprise or consist of the amino acid sequence of SEQ ID NO: 20. The nucleotide sequence encoding the F8 diabody is found in SEQ ID NO: 9.

A diabody for use in the invention may have at least 70%, more preferably one of at least 75%, 80%, 85%, 90%, 95%, 96%, 97%, 98%, 99% or 100%, sequence identity to the amino acid sequence of the F8 diabody set forth in SEQ ID NO: 20. It may be encoded by a nucleotide sequence having at least 70%, more preferably one of at least 75%, 80%, 85%, 90%, 95%, 96%, 97%, 98%, 99% or 100%, sequence identity to the nucleotide sequence set forth in SEQ ID NO: 9.

Preferably, the specific binding member comprises the CDRs, VH and/or VL domains, or the sequence of the F8 antibody.

The conjugate of the present invention comprises interleukin-4 (IL4). IL4 is preferably human IL4. IL4 may comprise or consist of the sequence shown in SEQ ID NO: 54. Typically, IL4 has at least 70%, more preferably one of at least 75%, 80%, 85%, 90%, 95%, 96%, 97%, 98%, 99% or 100%, sequence identity to the amino acid sequence shown in SEQ ID NO: 54. IL4 may be encoded by a nucleotide sequence having least 70%, more preferably one of at least 75%, 80%, 85%, 90%, 95%, 96%, 97%, 98%, 99% or 100%, sequence identity to SEQ ID NO: 53. The inventors have shown that substitution of the asparagine residue at position 38 of SEQ ID NO:54 with glutamine prevents glycosylation of IL4 at this residue. Substitution of the asparagine residue at position 38 of SEQ ID NO:54 with serine or alanine is expected to similarly prevent glycosylation of IL4. It is generally preferable to avoid glycosylation, as glycosylation may interfere with conjugate production, including batch consistency, and result in more rapid clearance of the conjugate from the patient's body. Preferably, a conjugates of the present invention, and in particular the IL4 present in a conjugate of the present invention, is not glycosylated. Thus, IL4 may comprise or consist of the sequence shown in SEQ ID NO: 54, except that the residue at position 38 of SEQ ID NO: 54 is a serine, glutamine, or alanine residue rather than an asparagine residue. Preferably, IL4 comprises or consists of the sequence shown in SEQ ID NO: 54, except that the residue at position 38 of SEQ ID NO: 54 is a glutamine residue rather than an asparagine residue. This sequence is shown in SEQ ID NO: 67. Alternatively, IL4 may comprise or consist of the sequence shown in SEQ ID NO: 54, except that the residue at position 38 of SEQ ID NO: 54 is a serine residue rather than an asparagine residue. As a further alternative, IL4 may comprise or consist of the sequence shown in SEQ ID NO: 54, except that the residue at position 38 of SEQ ID NO: 54 is an alanine residue rather than an asparagine residue. Occasionally IL4 may also be glycosylated at position 105 of SEQ ID NO:54. Thus, in addition to the mutations mentioned above, the residue at position 105 of SEQ ID NO: 54 may be a serine, glutamine, or alanine residue rather than an asparagine residue, in order to prevent glycosylation at this position.

IL4 in conjugates of the invention retains a biological activity of IL4, e.g. anti-inflammatory activity; the ability to inhibit cell proliferation and/or differentiation; the ability to induce apoptosis; the ability to stimulate the proliferation of activated B cells and T cells; the ability to induce the differentiation of naïve helper T cells into Th2 cells after antigen challenge; the ability to stimulate the proliferation of NK cells; the ability to up-regulate MHC class II production; and/or the ability to inhibit tumour growth and/or metastasis.

The peptide linker linking the specific binding member and IL4 may be a flexible peptide linker. Suitable examples of peptide linker sequences are known in the art. The linker may be 10-20 amino acids, preferably 15-20 amino acids in length. Most preferably, the linker is 15 amino acids in length. Most preferably, the linker has the sequence SSSSGSSSSGSSSSG (SEQ ID NO: 24).

In a preferred embodiment, the conjugate of the present invention may comprise or consist of the sequence shown in SEQ ID NO: 22 (F8-[human]IL4). The conjugate may have at least 70%, more preferably one of at least 75%, 80%, 85%, 90%, 95%, 96%, 97%, 98%, 99% or 100%, sequence identity to the amino acid sequence shown in SEQ ID NO: 22. The conjugate may be encoded by a nucleotide sequence having least 70%, more preferably one of at least 75%, 80%, 85%, 90%, 95%, 96%, 97%, 98%, 99% or 100%, sequence identity to SEQ ID NO: 11.

In another preferred embodiment, the conjugate of the present invention may comprise or consist of the sequence shown in SEQ ID NO: 68 (F8-[human]IL4 N284Q). The conjugate may have at least 70%, more preferably one of at least 75%, 80%, 85%, 90%, 95%, 96%, 97%, 98%, 99% or 100%, sequence identity to the amino acid sequence shown in SEQ ID NO: 68, provided the IL4 is not glycosylated.

The present inventors have shown that the conjugates of the present invention can be used in the treatment of various conditions and diseases, in particular conditions and diseases which are characterised by expression of the ED-A isoform of fibronectin, the ED-B isoform of fibronectin, and/or alternatively spliced tenascin C. Expression in this context may refer to over-expression compared with expression of the protein in normal tissue.

In another aspect, the present invention therefore relates to a conjugate according to the present invention for use in a method for treatment of the human or animal body by therapy, wherein the method comprises administering the conjugate to the patient (typically a human patient). Treatment, as referred to herein, may include prophylactic treatment and/or prevention. The present invention also provides methods of treatment comprising administering a conjugate of the invention, for example a pharmaceutical composition comprising such a conjugate, for the treatment of a condition or disease, and a method of making a medicament or pharmaceutical composition comprising formulating the conjugate of the present invention with a physiologically acceptable carrier or excipient.

The ED-A isoform of fibronectin, the ED-B isoform of fibronectin, and alternatively spliced tenascin C are associated with neoplastic growth and/or angiogenesis. Accordingly, a conjugate according to the present invention may be used in a method of inhibiting angiogenesis in a patient by targeting IL4 to the neovasculature in vivo. A conjugate according to the present invention may also be used in a method of delivering IL4 to sites of neovasculature, which are the result of angiogenesis and/or tissue remodelling, in a patient. A method of inhibiting angiogenesis by targeting IL4 to sites of neovasculature in a patient, the method comprising administering a therapeutically effective amount of a conjugate according to the present invention, and a method of delivering a IL4 to sites of neovasculature, which are the result of angiogenesis, in a human or animal comprising administering to the human or animal a specific binding member according to according to the present invention, are also provided. Further provided is the use of a conjugate of the present invention for the preparation, or manufacture, of a medicament for inhibiting angiogenesis, as well as the use of a conjugate of the present invention for the preparation, or manufacture, of a medicament for delivering IL4 to sites of neovasculature which are the result of angiogenesis, in a patient.

The present inventors have also surprisingly shown that that conjugates comprising IL4 and a specific binding member which binds an extra-cellular matrix component associated with neoplastic growth and/or angiogenesis are capable of treating rheumatoid arthritis with high efficacy. Conjugates comprising IL4 and a specific binding member which binds an extra-cellular matrix component associated with neoplastic growth and/or angiogenesis were found to be capable of treating rheumatoid arthritis with at least the same efficacy as the TNF inhibitor, TNFR-Fc. TNF inhibitors, such as Enbrel™ and Humeira™ represent the standard of care in rheumatoid arthritis patients.

The present invention therefore further relates to a conjugate according to the present invention for use in a method of treating an inflammatory autoimmune disease. A conjugate according to the present invention may also be used in a method of delivering IL4 to the sites of inflammatory autoimmune disease in a patient. A method of treating of an inflammatory autoimmune disease in a patient, the method comprising administering a therapeutically effective amount of a conjugate according to the present invention, and a method of delivering IL4 to the neovasculature of sites of inflammatory autoimmune disease in a human or animal comprising administering to the human or animal a specific binding member according to the present invention, are also provided. Further provided is the use of a conjugate of the present invention for the preparation, or manufacture, of a medicament for treating an inflammatory autoimmune disease in a patient, as well as the use of a conjugate of the present invention for the preparation, or manufacture, of a medicament for delivering IL4 to sites of inflammatory autoimmune disease in a patient.

The present inventors have also surprisingly shown that the conjugates of the invention were able to entirely eliminate rheumatoid arthritis symptoms, including paw swelling and arthritic score, in a mouse model of aggressive rheumatoid arthritis when administered in combination with a glucocorticoid. Such a complete suppression of rheumatoid arthritis symptoms has, to our knowledge, never before been seen in a model of aggressive rheumatoid arthritis. The reduction in the arthritic score and paw swelling, as well as weight loss, observed in mice treated with a conjugate of the invention and a glucocorticoid was also significantly greater than the reduction in these symptoms seen in mice treated with either a conjugate according to the invention or a glucocorticoid alone. This in itself is surprising, as combination treatment does not always result in a significant improvement over monotherapy. For example, the reduction in rheumatoid arthritis symptoms in mice treated with a combination of a conjugate of the invention and a conjugate comprising IL10 was only moderately greater than the reduction seen in mice treated with only a conjugate according of the present invention.

Thus, further provided is a conjugate according to the present invention for use in a method of treating an inflammatory autoimmune disease, wherein the method comprises administering the conjugate and glucocorticoid to an individual in need thereof. Also provided is a glucocorticoid for use in a method of treating an inflammatory autoimmune disease, wherein the method comprises administering the glucocorticoid and a conjugate according to the present invention to an individual in need thereof.

A glucocorticoid, as referred to herein, may be dexamethasone, cortisol, cortisone, prednisone, prednisolone, methylprednisolone, betamethasone, triamcinolone, beclometasone, fludrocortisone, deoxycorticosterone, or aldosterone. Preferably, the glucocorticoid is dexamethasone.

The present invention also relates to a kit comprising a conjugate according to the present invention and a glucocorticoid, wherein the conjugate may be for the treatment of an inflammatory autoimmune disease, as well as a method of treating an inflammatory autoimmune disease, the method comprising administering a conjugate according to the present invention and a glucocorticoid to an individual in need thereof.

The inflammatory autoimmune disease may be any inflammatory autoimmune disease which is characterized by expression of the ED-A isoform of fibronectin, the ED-B isoform of fibronectin, and/or alternatively spliced tenascin C, in particular at sites of inflammation in the patient. Preferably, the inflammatory autoimmune disease is rheumatoid arthritis (RA), multiple sclerosis (MS), inflammatory bowel disease (IBD), psoriasis, psoriatic arthritis, peridontitis, endometriosis, Behçet's disease, autoimmune insulitis, or autoimmune diabetes (such as diabetes mellitus type 1). The inflammatory autoimmune disease may be selected from the group of rheumatoid arthritis (RA), multiple sclerosis (MS), inflammatory bowel disease (IBD), psoriasis, psoriatic arthritis, endometriosis, Behçet's disease or peridontitis. Preferably, the inflammatory autoimmune disease is RA, MS, psoriasis, endometriosis, or autoimmune diabetes (such as diabetes mellitus type 1). More preferably, the inflammatory autoimmune disease is RA, MS, psoriasis, or endometriosis. The inflammatory autoimmune disease may be RA or psoriasis. The inflammatory autoimmune disease may be RA. The inflammatory autoimmune disease may be psoriasis. The inflammatory autoimmune disease may be endometriosis. The inflammatory autoimmune disease may be MS. The inflammatory autoimmune disease may be autoimmune diabetes (such as diabetes mellitus type 1). The inflammatory autoimmune disease may be Behçet's disease.

In particular, the present invention relates to a conjugate according to the present invention for use in a method of treating rheumatoid arthritis, and a conjugate according to the present invention for use in a method of delivering IL4 to the neovasculature of sites of rheumatoid arthritis in a patient. Also provided are a method of treating rheumatoid arthritis in a patient, the method comprising administering a therapeutically effective amount of a conjugate according to the present invention to the patient, and a method of delivering IL4 to the neovasculature of sites of rheumatoid arthritis in a human or animal comprising administering to the human or animal a specific binding member according to the present invention. Further provided is the use of a conjugate of the present invention for the preparation, or manufacture, of a medicament for treating rheumatoid arthritis in a patient, as well as the use of a conjugate of the present invention for the preparation, or manufacture, of a medicament for delivering IL4 to sites of rheumatoid arthritis in a patient.

Also provided is a conjugate according to the present invention for use in a method of treating rheumatoid arthritis, wherein the method comprises administering the conjugate and glucocorticoid to an individual in need thereof. Also provided is a glucocorticoid for use in a method of treating rheumatoid arthritis, wherein the method comprises administering the glucocorticoid and a conjugate according to the present invention to an individual in need thereof.

The present invention also relates to a kit comprising a conjugate according to the present invention and a glucocorticoid, wherein the conjugate may be for the treatment of rheumatoid arthritis, as well as a method of treating rheumatoid arthritis, the method comprising administering a conjugate according to the present invention and a glucocorticoid to an individual in need thereof.

The present invention also relates to a conjugate according to the present invention for use in a method of treating psoriasis, and a conjugate according to the present invention for use in a method of delivering IL4 to the neovasculature of sites of psoriasis in a patient. Also provided area method of treating psoriasis in a patient, the method comprising administering a therapeutically effective amount of a conjugate according to the present invention to the patient, and a method of delivering IL4 to the neovasculature of sites of psoriasis in a human or animal comprising administering to the human or animal a specific binding member according to the present invention. Further provided is the use of a conjugate of the present invention for the preparation, or manufacture, of a medicament for treating psoriasis in a patient, as well as the use of a conjugate of the present invention for the preparation, or manufacture, of a medicament for delivering IL4 to sites of psoriasis in a patient. The site of psoriasis may be psoriatic tissue.

Surprisingly, the present inventors have also shown that treatment with a conjugate comprising IL4 and a specific binding member which binds an extra-cellular matrix component associated with neoplastic growth and/or angiogenesis can be used to treat MS. Specifically, the present inventors have demonstrated that treatment with such a conjugate not only significantly reduced the severity of experimental autoimmune encephalomyelitis (EAE) in mice (a mouse model for MS) but was as effective as fingolimod, the gold standard for MS treatment, in treating EAE with the added advantage that the conjugate only needed to be administered every third day, compared with the daily administration required for fingolimod.

The present invention thus also relates to a conjugate according to the present invention for use in a method of treating MS, and a conjugate according to the present invention for use in a method of delivering IL4 to the neovasculature of sites of MS in a patient. Also provided are a method of treating MS in a patient, the method comprising administering a therapeutically effective amount of a conjugate according to the present invention to the patient, and a method of delivering IL4 to the neovasculature of sites of MS in a human or animal comprising administering to the human or animal a specific binding member according to the present invention. Further provided is the use of a conjugate of the present invention for the preparation, or manufacture, of a medicament for treating MS in a patient, as well as the use of a conjugate of the present invention for the preparation, or manufacture, of a medicament for delivering IL4 to sites of MS in a patient.

The present inventors have also found that conjugates comprising IL4 and a specific binding member which binds an extra-cellular matrix component associated with neoplastic growth and/or angiogenesis can be used to treat endometriosis. This was particularly surprising, as IL4 had previously shown to play a role in the progression of endometriosis (OuYang et al. 2008; OuYang et al. 2010). Specifically, OuYang et al. (2008) discloses in vitro experiments demonstrating that the proliferation of endometriotic stromal cells (ESCs) induced by locally produced IL-4 is involved in the development of endometriosis (see abstract and discussion). A later paper from the same group, OuYang et al. (2010), further shows that IL-4 induces eotaxin expression in ESCs in vitro, and postulates that IL4 may promote angiogenesis and the subsequent development of endometriosis. Based on these finding, the authors suggest that IL4 represents a possible target for anti-angiogenic therapy in the treatment of endometriosis (see abstract and discussion). In contrast, in vivo experiments performed by the present inventors show that administration of a conjugate comprising IL4 and a specific binding member which binds an extra-cellular matrix component associated with neoplastic growth and/or angiogenesis reduced both the volume and number of endometriotic lesions and in some cases was even capable of completely curing the disease. Administration of a control antibody conjugate comprising IL4 had no significant effect on the endometrial lesions, demonstrating that targeting of IL4 to the endometriotic tissue through the use of a binding member which binds an extra-cellular matrix component associated with neoplastic growth and/or angiogenesis is needed in order for a therapeutic effect to be observed.

Thus, the present invention relates to a conjugate according to the present invention for use in a method of treating endometriosis, and a conjugate according to the present invention for use in a method of delivering IL4 to the neovasculature of sites of endometriosis in a patient. Also provided are a method of treating endometriosis in a patient, the method comprising administering a therapeutically effective amount of a conjugate according to the present invention to the patient, and a method of delivering IL4 to the neovasculature of sites of endometriosis in a human or animal comprising administering to the human or animal a specific binding member according to the present invention. Further provided is the use of a conjugate of the present invention for the preparation, or manufacture, of a medicament for treating endometriosis in a patient, as well as the use of a conjugate of the present invention for the preparation, or manufacture, of a medicament for delivering IL4 to sites of endometriosis in a patient. The site of endometriosis may be endometrial tissue, such as endometrial lesions.

The present inventors have also surprisingly shown that conjugates comprising IL4 and a specific binding member which binds an extra-cellular matrix component associated with neoplastic growth and/or angiogenesis are capable of potently inhibiting tumour growth in three different syngeneic immunocompetent models of cancer. Previous studies with untargeted interleukin 4 could not achieve high enough concentrations of the cytokine at the site of malignancy in cancer patients at the doses tested due to toxicity of IL4. Based on the results disclosed in the present application, use of the conjugates of the present invention is expected to overcome this problem. In particular, the data in the present application shows that IL4 can be delivered to the tumour site using the conjugates of the invention. This is not possible for all cytokines.

In addition, the conjugates of the invention were found to be very well tolerated and to mediate durable cancer eradication when used in combination with conjugates comprising IL2 or IL12 and a specific binding member which binds an extra-cellular matrix component associated with neoplastic growth and/or angiogenesis. The synergistic effect observed when IL4-based conjugates were administered in combination with IL12-based conjugates was particularly surprising as these two cytokines are thought to mediate opposite effects on the regulation of T cell activity.

Thus, in another aspect, the present invention relates to a conjugate according to the present invention for use in a method of treating cancer by targeting IL4 to the neovasculature in vivo, and a conjugate according to the present invention for use in a method of delivering IL4 to the tumour neovasculature in a patient. A method of treating cancer by targeting IL4 to the neovasculature in a patient, the method comprising administering a therapeutically effective amount of a conjugate according to the present invention and a method of delivering IL4 to the tumour neovasculature in a human or animal comprising administering to the human or animal a specific binding member according to the present invention, are also provided. Further provided is the use of a conjugate of the present invention for the preparation, or manufacture, of a medicament for treating cancer in a patient, as well as the use of a conjugate of the present invention for the preparation, or manufacture, of a medicament for delivering IL4 to the tumour neovasculature in a patient. Also provided are a conjugate according to the present invention for use in a method of treating cancer comprising administering the conjugate and a second conjugate to an individual in need thereof, wherein the second conjugate comprises interleukin-12 (IL12), or interleukin-2 (IL2), and a specific binding member which binds an extra-cellular matrix component associated with neoplastic growth and/or angiogenesis; and a second conjugate comprising IL12, or IL2, and a specific binding member which binds an extra-cellular matrix component associated with neoplastic growth and/or angiogenesis for use in a method of treating cancer comprising administering the conjugate and a first conjugate according to the present invention to an individual in need thereof.

A kit comprising a conjugate according to the present invention and a second conjugate comprising IL12, or IL2, and a specific binding member which binds an extra-cellular matrix component associated with neoplastic growth and/or angiogenesis, wherein the conjugates are for treatment of cancer, and a method of treating cancer comprising administering a conjugate according to the present invention and a second conjugate comprising IL12, or IL2, and a specific binding member which binds an extra-cellular matrix component associated with neoplastic growth and/or angiogenesis to an individual in need thereof.

The second conjugate may comprise an scFv or be a diabody. The second conjugate may comprise a single chain diabody conjugated to IL12 or IL2. The second conjugate may bind the same or a different extra-cellular matrix component associated with neoplastic growth and/or angiogenesis than the conjugate of the invention. The second conjugate may bind a different epitope on an extra-cellular matrix component associated with neoplastic growth and/or angiogenesis than the conjugate of the invention.

The second conjugate may comprise a specific binding member, as described herein. In particular, the second conjugate may comprise a specific binding member that binds fibronectin or tenascin C. For example, the second conjugate may comprise a specific binding member that binds the Extra Domain-A (ED-A) isoform, Extra Domain-B (ED-B) isoform of fibronectin, or tenascin C. Preferably, the second conjugate comprises a specific binding member that binds the ED-A or ED-B of fibronectin, or binds the A1 domain of tenascin C. Most preferably, the second conjugate comprises a specific binding member that binds the ED-A of fibronectin.

The second conjugate may comprise a specific binding member which comprises an antigen binding site having the complementarity determining regions (CDRs) of antibody F8 set forth in SEQ ID NOs 12-17. The second conjugate may comprise a specific binding member which comprises the VH and VL domains of antibody F8 set forth in SEQ ID NOs 18 and 19. The second conjugate may comprise a specific binding member which comprises the amino acid sequence of antibody F8 set forth in SEQ ID NO: 20.

Alternatively, the second conjugate may comprise a specific binding member which comprises an antigen binding site having the complementarity determining regions (CDRs) of antibody L19 set forth in SEQ ID NOs 25-30. The second conjugate may comprise a specific binding member which comprises the VH and VL domains of antibody L19 set forth in SEQ ID NOs 31 and 32. The second conjugate may comprise a specific binding member which comprises the amino acid sequence of antibody L19 in scFv format set forth in SEQ ID NO: 33 or the amino acid sequence of antibody L19 in diabody format set forth in SEQ ID NO: 59 or SEQ ID NO: 62.

As a further alternative, the second conjugate may comprise a specific binding member which comprises an antigen binding site having the complementarity determining regions (CDRs) of antibody F16 set forth in SEQ ID NOs 34-39. The second conjugate may comprise a specific binding member which comprises the VH and VL domains of antibody F16 set forth in SEQ ID NOs 40 and 41. The second conjugate may comprise a specific binding member which comprises the amino acid sequence of antibody F16 in scFv format set forth in SEQ ID NO: 42 or the amino acid sequence of antibody F16 in diabody format set forth in SEQ ID NO: 60.

Preferably, the second conjugate comprises a specific binding member which comprises the CDRs, VH and/or VL domains, or the sequence of the F8 antibody. Preferably, the second conjugate comprises a specific binding member which is a diabody. The second conjugate may be a single chain fusion protein.

The second conjugate comprises IL2 or IL12. IL2 and IL12 are preferably human IL2 and human IL12, respectively. IL2 may comprise or consist of the sequence of IL2 shown in SEQ ID NO: 56. Typically, IL2 has at least 70%, more preferably one of at least 75%, 80%, 85%, 90%, 95%, 96%, 97%, 98%, 99% or 100%, sequence identity to the amino acid sequence shown in SEQ ID NO: 56. IL2 may be encoded by a nucleotide sequence having least 70%, more preferably one of at least 75%, 80%, 85%, 90%, 95%, 96%, 97%, 98%, 99% or 100%, sequence identity to SEQ ID NO: 55. IL12 may comprise or consist of the sequence of IL12 shown in SEQ ID NO: 58. Typically, IL12 has at least 70%, more preferably one of at least 75%, 80%, 85%, 90%, 95%, 96%, 97%, 98%, 99% or 100%, sequence identity to the amino acid sequence shown in SEQ ID NO: 58. IL12 may be encoded by a nucleotide sequence having least 70%, more preferably one of at least 75%, 80%, 85%, 90%, 95%, 96%, 97%, 98%, 99% or 100%, sequence identity to SEQ ID NO: 57. IL2 and IL12 in conjugates for use in the invention retain a biological activity of IL2 or IL12, respectively, e.g. the ability to inhibit tumour growth and/or metastasis.

The second conjugate of the present invention may comprise or consist of the sequence shown in SEQ ID NO: 50 (F8-[human]IL2). The conjugate may have at least 70%, more preferably one of at least 75%, 80%, 85%, 90%, 95%, 96%, 97%, 98%, 99% or 100%, sequence identity to the amino acid sequence shown in SEQ ID NO: 50. The conjugate may be encoded by a nucleotide sequence having least 70%, more preferably one of at least 75%, 80%, 85%, 90%, 95%, 96%, 97%, 98%, 99% or 100%, sequence identity to SEQ ID NO: 49.

Alternatively, the second conjugate of the present invention may comprise or consist of the sequence shown in SEQ ID NO: 48 ([murine]IL12-F8-F8), except that the sequence of murine IL12 is replaced with the sequence of human IL12, as shown in SEQ ID NO: 58, for example. Such a conjugate is disclosed in WO2013/014149. The sequence of such a conjugate is shown in SEQ ID NO: 61. The conjugate may have at least 70%, more preferably one of at least 75%, 80%, 85%, 90%, 95%, 96%, 97%, 98%, 99% or 100%, sequence identity with such a sequence. The conjugate may be encoded by a nucleotide sequence having least 70%, more preferably one of at least 75%, 80%, 85%, 90%, 95%, 96%, 97%, 98%, 99% or 100%, sequence identity to SEQ ID NO: 47, except that the coding sequence for murine IL12 has been replaced with a sequence coding for human IL12, such as SEQ ID NO: 57.

Cancer and other tumours and neoplastic conditions which may be treated using the conjugates of the present invention whether in combination with a second conjugate as described above, or not, include cancers which express an isoform of fibronectin comprising domain ED-A or ED-B, or alternatively spliced tenascin-C comprising for example domain A1. Preferably the cancer expresses the ED-A isoform of fibronectin. For example, the conjugates of the present invention may be used to treat any type of solid or non-solid cancer or malignant lymphoma and especially germ cell cancer (such as teratocarcinoma), liver cancer, lymphoma (such as Hodgkin's or non-Hodgkin's lymphoma), leukaemia (e.g. acute myeloid leukaemia), skin cancer, melanoma, sarcoma (e.g. fibrosarcoma), bladder cancer, breast cancer, uterine cancer, ovarian cancer, prostate cancer, lung cancer, colorectal cancer, cervical cancer, head and neck cancer, oesophageal cancer, pancreatic cancer, renal cancer, stomach cancer and cerebral cancer. Cancers may be familial or sporadic. Cancers may be metastatic or non-metastatic. Preferably, the cancer is a cancer selected from the group consisting of germ cell cancer (such as teratocarcinoma); colorectal cancer; Hodgkin's or non-Hodgkin's lymphoma; melanoma; pancreatic cancer; soft tissue sarcoma; fibrosarcoma; or renal cell carcinoma. The cancer may be selected from the group consisting of germ cell cancers, such as teratocarcinoma; colorectal cancer; and lymphoma.

In some instances, patients requiring treatment are polymorbid. This is particularly the case where patients are elderly. The ability of the conjugates of the present invention to treat both cancer and inflammatory autoimmune diseases, such as rheumatoid arthritis, therefore makes them particularly suitable for treating such polymorbid patients.

Thus, in another aspect, the present invention relates to a conjugate according to the present invention for use in a method of treating cancer and rheumatoid arthritis in a patient; and a conjugate according to the present invention for use in a method of delivering IL4 to the tumour neovasculature and to the neovasculature of sites of rheumatoid arthritis in a patient. Also provided are a method of treating cancer and rheumatoid arthritis by targeting IL4 to the neovasculature in a patient, the method comprising administering a therapeutically effective amount of a conjugate according to the present invention to the patient, and a method of delivering IL4 to the tumour neovasculature and the neovasculature of sites of rheumatoid arthritis in a human or animal comprising administering to the human or animal a specific binding member according to the present invention. Further provided is the use of a conjugate of the present invention for the preparation, or manufacture, of a medicament for treating cancer and rheumatoid arthritis in a patient, as well as the use of a conjugate of the present invention for the preparation, or manufacture, of a medicament for delivering IL4 to the tumour neovasculature and sites of rheumatoid arthritis in a patient.

In another aspect, the present invention provides a conjugate according to the present invention for use in a method of treating, preventing, or delaying the onset of autoimmune insulitis or autoimmune diabetes in a patient, as well as a conjugate according to the present invention for use in a method of delivering IL4 to sites of autoimmune insulitis or autoimmune diabetes in a patient. Also provided are a method of treating, preventing, or delaying the onset of autoimmune insulitis or autoimmune diabetes in a patient, the method comprising administering a therapeutically effective amount of a conjugate according to the present invention to the patient, and a method of delivering IL4 to sites of autoimmune insulitis or autoimmune diabetes in a human or animal comprising administering to the human or animal a conjugate according to the present invention. Further provided is the use of a conjugate of the present invention for the preparation, or manufacture, of a medicament for treating, preventing, or delaying the onset of autoimmune insulitis or autoimmune diabetes in a patient, as well as the use of a conjugate of the present invention for the preparation, or manufacture, of a medicament for delivering IL4 to sites of autoimmune insulitis or autoimmune diabetes in a patient. Delivery of IL4 to sites of sites of autoimmune insulitis or autoimmune diabetes may refer to delivery of IL4 to the pancreas. Autoimmune diabetes may refer to diabetes mellitus type 1.

### BRIEF DESCRIPTION OF THE FIGURES

FIGS. 1A, B and C show the expression and in vitro characterization of the non-covalent dimer F8-IL4 (monomer 39.6 kDa, dimer 79.2 kDa). A: SDS-Page analysis (M=molecular marker; N=non-reducing; R=reducing conditions). B: Size exclusion chromatography profile. C: Surface plasmon analysis of F8-IL4 on an EDA coated sensor chip. The Surface Plasmon Resonance (SPR) signal (expressed in response units [RU]) is indicated on the left. The peak at 13.8 ml retention volume corresponds to the homodimer F8-IL4.

FIGS. 2A, B and C show the expression and in vitro characterization of covalent homodimeric muTNFR-Fc dimer. A: SDS-PAGE analysis of purified muTNFR-Fc (M=molecular marker; N=non-reducing; R=reducing conditions). B: Size exclusion chromatography (SEC200). C: Bioactivity assay of muTNFR-Fc. muTNFR-Fc inhibits TNF induced killing of mouse fibroblasts.

FIG. 3 shows the characterization of therapeutic potential of F8-murine IL4 in an aggressive model of collagen induced arthritis in the mouse. DBA/J1 mice immunized with bovine collagen/CFA were included in the therapy experiments when showing symptoms of arthritis (paw and/or toe swelling) and received intravenous injections of either TNFR-Fc (30 μg; diamonds), F8-IL4 (5 μg; triangles) F8-IL4 (100 μg; circles) or PBS (buffer vehicle; squares) on days 1, 4 and 7 (arrows indicate injection time points). A: The arthritic score was evaluated daily and results are expressed as the mean arthritic score (±standard error of the mean [SEM]) (n≧9). F8-IL4 in the high dose schedule exhibited a more potent disease-modulating effect than the murine version of Enbrel (TNFR-Fc) in this model of aggressive arthritis. B: shows changes in weight of treated mice compared to the weight at the start of therapy. Mice that received F8-IL4 at a dose of 100 μg/injection lost less weight than mice receiving only the buffer vehicle (PBS). This indicates that the therapy was well tolerated and mice were in a better general state of health. C: Paw swelling was measured daily and paw thickness is expressed as the mean of thickness of all four paws of each animal (±SEM). Mice treated with 100 μg of F8-IL4 had less severe swollen paws than mice in other treatment groups. D and E: F8-IL4 showed superior therapeutic activity than the untargeted control immunocytokine KSF-IL4 in reducing arthritic score and paw swelling. No synergistic effect was seen when F8-IL4 was administered in combination with TNFR-Fc.

FIG. 4A: shows the results of a bioactivity assay with CTLL2 cells (20000cells/well). EC₅₀: KSF-IL4 (20 pM), F8-IL4 (23 pM), recombinant IL4 (28 pM). B: shows the results of an immunofluorescence analysis of biotinylated F8-IL4 respectively KSF-IL4 on tumour sections (scale bar=100 μm). C: Monitoring of changes in weight of treated mice. Results are expressed as percentage of the weight on therapy start.

FIG. 5 shows targeting of F8-IL4 in F9 teratocarcinoma. A: Quantitative biodistribution study of radioiodinated F8-IL4 (black bars) respectively KSF-IL4 (grey bars). Mice bearing subcutaneous (s.c.) tumours were injected intravenously (i.v.) with 15 μg radiolabeled protein. Mice were sacrificed after 24 hours. Organs were excised and radioactivity counted, expressing results as percent of injected dose per gram of tissue (% ID/g±SE). B: Histochemical confirmation of targeting by the analysis of treated tumours. Mice received 3 injections of protein, 24 h later tumours were excised. Cryostat sections of tumours were stained with anti-IL4 antibody (Alexa488) and anti-CD31 antibody (Alexa594). Scale bar=100 μm. FIGS. 5A and B show that in contrast to the non-targeted KSF-IL4, the EDA targeting F8-IL4 conjugate accumulates in tumour tissue while sparing healthy tissue (tumour-to-blood ratio of 12). The low level of F8-IL4 in blood indicates fast clearance from the blood stream, which is favorable in terms of unwanted systemic effects.

FIG. 6 shows the therapeutic performance of F8-IL4 against F9 teratocarcinoma. A: Dose escalation study on F9 bearing mice. Treatment was started when tumours reached a volume of 70 mm³ and mice were injected every 48 h (indicated by arrows) with either PBS (×), 45 μg F8-IL4 (⋄) or 90 μg F8-IL4 (♦). Data represent mean tumour volumes (±SEM), n=4 mice per group. B: Comparison of targeted delivery of IL4 to non-targeted administration. Mice received i.v. injections of 90 μg F8-IL4 (♦), 90 μg KSF-IL4 (▴) or PBS (×) every second day (indicated by arrows). Data represent mean tumour volumes (±SEM), n=5 mice per group.

FIG. 7 shows the therapeutic activity of F8-IL4 in combination with F8-IL2 or F8-IL12 against F9 teratocarcinoma. A: Combination treatment of F8-IL4 with F8-IL2. When F9 tumours were clearly palpable, mice were randomly grouped and injected with PBS, 90 μg F8-IL4, 20 μg F8-IL2 or the combination of both (90 μg F8-IL4 plus 20 μg F8-IL2). Data represent mean tumour volumes (±SEM), n=5 mice per group. B: Mice bearing F9 tumours were injected with PBS, 90 μg F8-IL4, 8.75 μg F8-IL12 or the combination of the single agents (90 μg F8-IL4 plus 8.75 μg F8-IL12). Data represent mean tumour volumes (±SEM), n=5 mice per group. C: Weight monitoring of tumour-bearing mice treated with PBS, F8-IL4, F8-IL2, F8-IL12 and combinations thereof as indicated.

FIG. 8 shows ex vivo immunofluorescence analysis of tumour infiltrating cells on F9 tumour section following treatment with PBS, KSF-IL4, F8-IL4, F8-IL2, F8-IL4 in combination with F8-IL2, F8-IL12 or F8-IL4 in combination with F8-IL12. Scale bars, 100 μm.

FIG. 9 shows the anti-tumoural activity of targeted IL4 against CT26 colon carcinoma. A: Biodistribution study of radioiodinated F8-IL4 with CT26-tumour-bearing mice. Mice were sacrificed after 24 hours. Organs were excised and radioactivity counted, expressing results as percent of injected dose per gram of tissue (% ID/g±SE). B: Immunofluorescence analysis of biotinylated F8-IL4 respectively KSF-IL4 on tumour sections (scale bar=100 μm). C: Therapeutic comparison of F8-IL4 to KSF-IL4 (negative control, specific to egg lysozyme). Mice received i.v. injections of 90 μg F8-IL4 (♦), 90 μg KSF-IL4 (▴) or PBS (×) every 48 h. Data represent mean tumour volumes (±SEM), n=5 mice per group. D: Combination of F8-IL4 with F8-IL12 in the treatment of CT26 tumours. Mice were given 4 injections (every 48 h) of either PBS (×), 90 μg F8-IL4 (♦), 8.75 μg F8-IL12 (□) and the combination (90 μg F8-IL4 plus 8.75 μg F8-IL12) (▪) per injection. Data represent mean tumour volumes (±SEM), n=5 mice per group.

FIG. 10 shows the functional activity of F8-IL4 against A20 lymphoma. A: Quantitative Biodistribution study of radioiodinated F8-IL4 with A20-tumour-bearing mice. Mice were sacrificed 24 hours after the injection of 15 μg radioiodinated protein. Organs were excised and radioactivity counted. Results are expressed as percent of injected dose per gram of tissue (% ID/g±SE). B: Accumulation of biotinylated F8-IL4 respectively KSF-IL4 on A20 tumour sections (scale bar=100 μm). C: Comparison of targeted IL4 to non-targeted administration. Mice received i.v. injections of 90 μg F8-IL4, 90 μg KSF-IL4 or PBS every second day. Data represent mean tumour volumes (±SEM), n=5 mice per group. D: Therapeutic activity of F8-IL4 in combination with F8-IL12. Treatment was started when tumours reached a volume of 70 mm³ and mice were 4 times injected (every 48 h) of either PBS (×), 90 μg F8-IL4 (♦), 8.75 μg F8-IL12 (□) and the combination (90 μg F8-IL4 plus 8.75 μg F8-IL12) (▪) per injection. Data represent mean tumour volumes (±SEM), n=5 mice per group.

FIG. 11 shows the functional activity of F8-IL4 in an IMQ-induced inflammation model of psoriasis. A: Experiment timeline. Imiquimod-containing Aldara cream was applied to the ears of C57BL/6 mice were treated on days 1, 2, 3, 4, 5 and 7. Therapy was started on day 7 and repeated on days 9 and 11. Mice were sacrificed on day 13. B: Ear thickness of C57BL/6 mice on days 1 through to 13. Treatment was started on day 7 and repeated on days 9 and 11. Mice were injected with either PBS (), 100 ug SIP (F8) (▪), 30 ug murine TNFR-Fc (▴), 100 ug F8-IL4 (♦), or 100 ug KSF-IL4 (×). Results are expressed as ear thickness in μm±SEM. C: The change in ear thickness from initiation of treatment (day 7). Results are expressed as delta ear thickness in μm±SEM. D: Difference in the weight of the ear draining lymph nodes. Mice were sacrificed on day 13 and the ear draining lymph nodes were excised and weighted. E: Weight of mice undergoing treatment. Mice were weighed daily from initiation of treatment (day 7). No loss of weight was observed.

FIG. 12 shows a quantitative analysis of the biodistribution of SIP (F8) and F8-IL4 in tissue from mice with IMQ-induced inflammation in the ears. Mice were injected with 10 ug radioiodinated protein (I-125) and after 24 h mice were sacrificed and organs were excised. Results are expressed as % injected dose per gram A: Biodistribution analysis of SIP (F8). B: Biodistribution analysis of F8-IL4.

FIG. 13 shows the functional activity of F8-IL4 in a CHS-induced skin inflammation model of psoriasis. A: Experiment timeline. Heterozygous female VEGF-A transgenic mice were sensitized. Five days after sensitization the ears were challenged (day 0). Therapy was started on day 7. Mice were sacrificed on day 15. B: Ear thickness of mice on days 1, 7, 9, 11, 13 and 15. Treatment was started on day 7 and repeated on days 9, 11 and 13. Mice were injected with either PBS (), 100 ug SIP (F8) (▪), 30 ug murine TNFR-Fc(▴), 100 ug F8-IL4 (♦), or 100 ug KSF-IL4 (×). Results are expressed as ear thickness in μm±SEM. C: The change in ear thickness from initiation of treatment (day 7). Results are expressed as delta ear thickness in μm±SEM. D: Difference in the weight of the ear draining lymph nodes. Mice were sacrificed on day 15 and the ear draining lymph nodes were excised and weighted. E: Weight of mice undergoing treatment. Mice were weighed at the start of treatment (day 7) and days 9, 11, 13 and 15. No loss of weight was observed.

FIG. 14: Analysis of cytokine levels in tissue extracts from psoriasis models. A: Analysis of cytokine levels tissue of mice following treatment in an IMQ-induced inflammation mode of psoriasis. B: Analysis of cytokine levels tissue of mice following treatment in a CHS-induced ear inflammation model.

FIG. 15 shows treatment of rheumatoid arthritis in an aggressive model of collagen induced arthritis in the mouse using F8-IL4 in combination with dexamethasone or L19-IL10. DBA/J1 mice were immunized with bovine collagen/Complete Freund's Adjuvant (CFA). Mice used for experiments shown in FIG. 15 had a clinical score of 1 to 4 and received injections of F8-IL4 either subcutaneously (s.c.) (100 μg; diamonds), or intravenously (i.v.) (100 μg; triangles), or received dexamethasone (100 μg; circles), L19-IL10 (200 μg; indicated by “x”), F8-IL4 and dexamethasone (100 μg of each; crosses), or F8-IL4 and L19-IL10 (100 μg and 200 μg, respectively; filled squares), or PBS (empty squares). F8-IL4 and L19-IL10 injections were given on days 1, 3 and 7. Dexamethasone injections were given daily until day 9. A: shows the arthritic score for the treated mice. The arthritic score was evaluated daily and results are expressed as the mean arthritic score (+standard error of the mean [SEM]). Treatment with a combination of F8-IL4 and dexamethasone exhibited a more potent disease-modulating effect than treatment with either F8-IL4 or dexamethasone alone. Treatment with a combination of F8-IL4 and L19-IL10 also exhibited a more potent disease-modulating effect than treatment with L19-IL10 alone. B: Paw swelling was measured daily and paw thickness is expressed as the mean of thickness of all four paws of each animal (+SEM). Mice treated with a combination of F8-IL4 and dexamethasone exhibited less paw swelling than mice treated with either F8-IL4 or dexamethasone alone. Mice treated with a combination of F8-IL4 and L19-IL10 also exhibited less paw swelling than treatment with L19-IL10 alone. The dashed line indicates a baseline thickness of 1.8 mm which represents the average paw thickness of healthy DBA/J1 mice. C: shows changes in weight of treated mice compared to the weight at the start of therapy. Mice treated with a combination of F8-IL4 and dexamethasone exhibited less weight loss than mice treated with F8-IL4 alone. Similarly, mice treated with a combination of F8-IL4 and L19-IL10 exhibited less weight loss than mice treated with either F8-IL4 or L19-IL10 alone. This indicates that both combination treatments were well tolerated.

FIG. 16 shows the anti-tumoural activity of L19-IL4 against Wehi 164 mouse fibrosarcoma. Mice were injected intravenously with L19-IL10 (▪), KSF-IL10 (▴), L19-IL4 (×), KSF-IL4 (*), or PBS (♦) every 48 h. Data represent mean tumour volumes (+SEM), n=3-4 mice per group. Results are shown as mean tumour weight (mg) over time (days).

FIG. 17 shows that F8-IL4 can be used to treat endometriosis in mice. A: the volume [cm³] of endometriotic lesions was significantly reduced in mice treated using F8-IL4 compared with mice who received PBS. B: the number of endometriotic lesions was also significantly reduced in mice treated with F8-IL4 compared with mice who received PBS. In three mice out of ten, the administration of F8-IL4 resulted in a complete cure of the disease. In contrast, treatment of mice with KSF-IL4 (specific to egg lysozyme) showed no effect on either the volume or number of endometriotic lesions compared with the control mice treated with PBS.

FIG. 18 shows that F8-IL4 can be used to treat experimental autoimmune encephalomyelitis (EAE), a model for multiple sclerosis, in mice. EAE severity in mice treated using F8-IL4 (filled circles) was significantly reduced compared with mice who received PBS (vehicle; filled diamonds). F8-IL4 was also as efficacious at treating EAE as fingolimod (filled squares), the gold standard for treatment of MS. *=P<0.05, n.s.=not significant (as determined by two-way ANOVA followed by Bonferroni correction).

FIG. 19 shows that IL4 can be targeted to the perivascular space in the pancreas of diabetic mice. CD31⁺ blood vessels (filled white arrows) colocalize with EDA (open white arrows) in the pancreas of diabetic mice. No such colocalization was seen when cells were stained using KSF antibody, which is specific to egg lysozyme, or in the absence of a primary antibody (see “no antibody” in FIG. 19).

FIG. 20 shows that wild-type F8-human IL4 (F8-hIL4) is glycosylated, while the mutant F8-hIL4 N284Q is not. A and B show the integrated and deconvoluted mass spectra for wild-type F8-hIL4, respectively. C and D show the integrated and deconvoluted mass spectra for the F8-hIL4 N284Q mutant, respectively. The deconvoluted spectrum for wild-type F8-hIL4 (B) shows two major peaks at 43289.6 and 42998.5 Da, which are significantly higher than the expected mass of wild-type F8-hIL4 with five disulfide bonds (40938.0 Da), indicating the presence of N-linked glycosylation. In contrast, the deconvoluted spectrum of the F8-hIL4 N284Q mutant (D) displaying only a single peak at 40951.4 Da, corresponds with the theoretical mass of the F8-hIL4 N284Q mutant with five disulfide bonds (40952.0 Da), demonstrating that the mutant is not glycosylated. Common ESI adduct peaks are indicated with an asterisk (*) in FIG. 20.

FIG. 21 shows that wild-type F8-hIL4 and the F8-hIL4-N284Q mutant have comparable targeting properties in vivo. A: in vivo targeting performances of wild-type F8-hIL4. B: in vivo targeting performances of F8-hIL4-N284Q. FIG. 21 shows the percentage injected dose per gram of tissue 24 hours after intravenous administration of the respective antibodies (% ID/g+SE).

### TERMINOLOGY

Conjugate

A conjugate may comprise a specific binding member and an interleukin, such as IL4. IL2 or IL12. The specific binding member is preferably an antibody, most preferably a diabody, as described herein. Where the conjugate comprises a diabody, one or both of the single chain Fvs (scFvs) of the diabody may be conjugated to the interleukin, e.g. IL4. An scFv may be conjugated to the interleukin, such as IL4, by means of a peptide linker, allowing the scFv-interleukin construct to be expressed as a fusion protein. By “fusion protein” is meant a polypeptide that is a translation product resulting from the fusion of two or more genes or nucleic acid coding sequences into one open reading frame (ORF). The fused expression products of the two genes in the ORF may be conjugated by a peptide linker encoded in-frame. Suitable peptide linkers are described herein.

Specific Binding Member

This describes one member of a pair of molecules that bind specifically to one another. The members of a specific binding pair may be naturally derived or wholly or partially synthetically produced. One member of the pair of molecules has an area on its surface, or a cavity, which binds to and is therefore complementary to a particular spatial and polar organization of the other member of the pair of molecules. Examples of types of binding pairs are antigen-antibody, biotin-avidin, hormone-hormone receptor, receptor-ligand, enzyme-substrate. The present invention is concerned with antigen-antibody type reactions.

A specific binding member normally comprises a molecule having an antigen-binding site. For example, a specific binding member may be an antibody molecule or a non-antibody protein that comprises an antigen-binding site. A specific binding member, as referred to herein, is preferably an antibody molecule.

An antigen binding site may be provided by means of arrangement of complementarity determining regions (CDRs) on non-antibody protein scaffolds such as fibronectin or cytochrome B etc. (Haan & Maggos, (2004), BioCentury, 12(5): A1-A6; Koide et al., (1998). Journal of Molecular Biology, 284: 1141-1151; Nygren et al., (1997), Current Opinion in Structural Biology, 7: 463-469), or by randomizing or mutating amino acid residues of a loop within a protein scaffold to confer binding specificity for a desired target. Scaffolds for engineering novel binding sites in proteins have been reviewed in detail by Nygren et al. (1997) (Current Opinion in Structural Biology, 7: 463-469). Protein scaffolds for antibody mimics are disclosed in WO/0034784, in which the inventors describe proteins (antibody mimics) that include a fibronectin type III domain having at least one randomized loop. A suitable scaffold into which to graft one or more CDRs, e.g. a set of HCDRs, may be provided by any domain member of the immunoglobulin gene superfamily. The scaffold may be a human or non-human protein. An advantage of a non-antibody protein scaffold is that it may provide an antigen-binding site in a scaffold molecule that is smaller and/or easier to manufacture than at least some antibody molecules. Small size of a specific binding member may confer useful physiological properties such as an ability to enter cells, penetrate deep into tissues or reach targets within other structures, or to bind within protein cavities of the target antigen. Use of antigen binding sites in non-antibody protein scaffolds is reviewed in Wess, 2004, In: BioCentury, The Bernstein Report on BioBusiness, 12(42), A1-A7. Typical are proteins having a stable backbone and one or more variable loops, in which the amino acid sequence of the loop or loops is specifically or randomly mutated to create an antigen-binding site that binds the target antigen. Such proteins include the IgG-binding domains of protein A from S. aureus, transferrin, tetranectin, fibronectin (e.g. 10th fibronectin type III domain) and lipocalins. Other approaches include synthetic “Microbodies” (Selecore GmbH), which are based on cyclotides—small proteins having intra-molecular disulphide bonds.

In addition to antibody sequences and/or an antigen-binding site, a specific binding member for use in the present invention may comprise other amino acids, e.g. forming a peptide or polypeptide, such as a folded domain, or to impart to the molecule another functional characteristic in addition to ability to bind antigen.

For example, a specific binding member may comprise a catalytic site (e.g. in an enzyme domain) as well as an antigen binding site, wherein the antigen binding site binds to the antigen and thus targets the catalytic site to the antigen. The catalytic site may inhibit biological function of the antigen, e.g. by cleavage.

Although, as noted, CDRs can be carried by non-antibody scaffolds, the structure for carrying a CDR or a set of CDRs will generally be an antibody heavy or light chain sequence or substantial portion thereof in which the CDR or set of CDRs is located at a location corresponding to the CDR or set of CDRs of naturally occurring VH and VL antibody variable domains encoded by rearranged immunoglobulin genes. The structures and locations of immunoglobulin variable domains may be determined by reference to Kabat et al. (1987) (Sequences of Proteins of Immunological Interest. 4^{th} Edition. US Department of Health and Human Services.), and updates thereof, now available on the Internet (at immuno.bme.nwu.edu or find “Kabat” using any search engine).

By CDR region or CDR, it is intended to indicate the hypervariable regions of the heavy and light chains of the immunoglobulin as defined by Kabat et al. (1987) Sequences of Proteins of Immunological Interest, 4^{th} Edition, US Department of Health and Human Services (Kabat et al., (1991a), Sequences of Proteins of Immunological Interest, 5^{th} Edition, US Department of Health and Human Services, Public Service, NIH, Washington, and later editions). An antibody typically contains 3 heavy chain CDRs and 3 light chain CDRs. The term CDR or CDRs is used here in order to indicate, according to the case, one of these regions or several, or even the whole, of these regions which contain the majority of the amino acid residues responsible for the binding by affinity of the antibody for the antigen or the epitope which it recognizes.

Among the six short CDR sequences, the third CDR of the heavy chain (HCDR3) has a greater size variability (greater diversity essentially due to the mechanisms of arrangement of the genes which give rise to it). It can be as short as 2 amino acids although the longest size known is 26. Functionally, HCDR3 plays a role in part in the determination of the specificity of the antibody (Segal et al., (1974), PNAS, 71:4298-4302; Amit et al., (1986), Science, 233:747-753; Chothia et al., (1987), J. Mol. Biol., 196:901-917; Chothia et al., (1989), Nature, 342:877-883; Caton et al., (1990), J. Immunol., 144:1965-1968; Sharon et al., (1990a), PNAS, 87:4814-4817; Sharon et al., (1990b), J. Immunol., 144:4863-4869; Kabat et al., (1991b), J. Immunol., 147:1709-1719).

Antibody Molecule

This describes an immunoglobulin whether natural or partly or wholly synthetically produced. The term also relates to any polypeptide or protein comprising an antibody antigen-binding site. It must be understood here that the invention does not relate to the antibodies in natural form, that is to say they are not in their natural environment but that they have been able to be isolated or obtained by purification from natural sources, or else obtained by genetic recombination, or by chemical synthesis, and that they can then contain unnatural amino acids as will be described later. Antibody fragments that comprise an antibody antigen-binding site include, but are not limited to, antibody molecules such as Fab, Fab′, Fab′-SH, scFv, Fv, dAb, Fd; and diabodies. A specific binding member, or antibody, for use in the present invention preferably comprises an scFv or is a diabody. Most preferably, a specific binding member, or antibody, for use in the present invention is a diabody.

It is possible to take monoclonal and other antibodies and use techniques of recombinant DNA technology to produce other antibodies or chimeric molecules that bind the target antigen. Such techniques may involve introducing DNA encoding the immunoglobulin variable region, or the CDRs, of an antibody to the constant regions, or constant regions plus framework regions, of a different immunoglobulin. See, for instance, EP-A-184187, GB 2188638A or EP-A-239400, and a large body of subsequent literature. A hybridoma or other cell producing an antibody may be subject to genetic mutation or other changes, which may or may not alter the binding specificity of antibodies produced.

As antibodies can be modified in a number of ways, the term “antibody molecule” should be construed as covering any specific binding member or substance having an antibody antigen-binding site with the required specificity and/or binding to antigen. Thus, this term covers antibody fragments and derivatives, including any polypeptide comprising an antibody antigen-binding site, whether natural or wholly or partially synthetic. Chimeric molecules comprising an antibody antigen-binding site, or equivalent, fused to another polypeptide (e.g. derived from another species or belonging to another antibody class or subclass) are therefore included. Cloning and expression of chimeric antibodies are described in EP-A-0120694 and EP-A-0125023, and a large body of subsequent literature.

Further techniques available in the art of antibody engineering have made it possible to isolate human and humanized antibodies. For example, human hybridomas can be made as described by Kontermann & Dubel (2001), S, Antibody Engineering, Springer-Verlag New York, LLC; ISBN: 3540413545. Phage display, another established technique for generating specific binding members has been described in detail in many publications such as WO92/01047 (discussed further below) and U.S. Pat. No. 5,969,108, U.S. Pat. No. 5,565,332, U.S. Pat. No. 5,733,743, U.S. Pat. No. 5,858,657, U.S. Pat. No. 5,871,907, U.S. Pat. No. 5,872,215, U.S. Pat. No. 5,885,793, U.S. Pat. No. 5,962,255, U.S. Pat. No. 6,140,471, U.S. Pat. No. 6,172,197, U.S. Pat. No. 6,225,447, U.S. Pat. No. 6,291,650, U.S. Pat. No. 6,492,160, U.S. Pat. No. 6,521,404 and Kontermann & Dubel (2001), S, Antibody Engineering, Springer-Verlag New York, LLC; ISBN: 3540413545. Transgenic mice in which the mouse antibody genes are inactivated and functionally replaced with human antibody genes while leaving intact other components of the mouse immune system, can be used for isolating human antibodies (Mendez et al., (1997), Nature Genet, 15(2): 146-156).

Synthetic antibody molecules may be created by expression from genes generated by means of oligonucleotides synthesized and assembled within suitable expression vectors, for example as described by Knappik et al. (2000) J. Mol. Biol. 296, 57-86 or Krebs et al. (2001) Journal of Immunological Methods, 254 67-84.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward et al. (1989) Nature 341, 544-546; McCafferty et al., (1990) Nature, 348, 552-554; Holt et al. (2003) Trends in Biotechnology 21, 484-490), which consists of a VH or a VL domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al. (1988) Science, 242, 423-426; Huston et al. (1988) PNAS USA, 85, 5879-5883); (viii) bispecific single chain Fv dimers (PCT/US92/09965) and (ix) “diabodies”, multivalent or multispecific fragments constructed by gene fusion (WO94/13804; Holliger et al. (1993a), Proc. Natl. Acad. Sci. USA 90 6444-6448). Fv, scFv or diabody molecules may be stabilized by the incorporation of disulphide bridges linking the VH and VL domains (Reiter et al. (1996), Nature Biotech, 14, 1239-1245). Minibodies comprising a scFv joined to a CH3 domain may also be made (Hu et al. (1996), Cancer Res., 56(13):3055-61). Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group.

Antibody fragments for use in the invention can be obtained starting from any of the antibody molecules described herein, e.g. antibody molecules comprising VH and/or VL domains or CDRs of any of antibodies described herein, by methods such as digestion by enzymes, such as pepsin or papain and/or by cleavage of the disulfide bridges by chemical reduction. In another manner, antibody fragments of the present invention may be obtained by techniques of genetic recombination likewise well known to the person skilled in the art or else by peptide synthesis by means of, for example, automatic peptide synthesizers such as those supplied by the company Applied Biosystems, etc., or by nucleic acid synthesis and expression.

Functional antibody fragments according to the present invention include any functional fragment whose half-life is increased by a chemical modification, especially by PEGylation, or by incorporation in a liposome.

A dAb (domain antibody) is a small monomeric antigen-binding fragment of an antibody, namely the variable region of an antibody heavy or light chain (Holt et al. (2003) Trends in Biotechnology 21, 484-490). VH dAbs occur naturally in camelids (e.g. camel, llama) and may be produced by immunizing a camelid with a target antigen, isolating antigen-specific B cells and directly cloning dAb genes from individual B cells. dAbs are also producible in cell culture. Their small size, good solubility and temperature stability makes them particularly physiologically useful and suitable for selection and affinity maturation. A specific binding member of the present invention may be a dAb comprising a VH or VL domain substantially as set out herein, or a VH or VL domain comprising a set of CDRs substantially as set out herein.

As used herein, the phrase “substantially as set out” refers to the characteristic(s) of the relevant CDRs of the VH or VL domain of specific binding members described herein will be either identical or highly similar to the specified regions of which the sequence is set out herein. As described herein, the phrase “highly similar” with respect to specified region(e)of one or more variable domains, it is contemplated that from 1 to about 5, e.g. from 1 to 4, including 1 to 3, or 1 or 2, or 3 or 4, amino acid substitutions may be made in the CDR and/or VH or VL domain.

Bispecific or bifunctional antibodies form a second generation of monoclonal antibodies in which two different variable regions are combined in the same molecule (Holliger and Bohlen 1999 Cancer and metastasis rev. 18: 411-419). Their use has been demonstrated both in the diagnostic field and in the therapy field from their capacity to recruit new effector functions or to target several molecules on the surface of tumor cells. Where bispecific antibodies are to be used, these may be conventional bispecific antibodies, which can be manufactured in a variety of ways (Holliger et al. (1993b), Current Opinion Biotechnol 4, 446-449), e.g. prepared chemically or from hybrid hybridomas, or may be any of the bispecific antibody fragments mentioned above. These antibodies can be obtained by chemical methods (Glennie et al., (1987) J. Immunol. 139, 2367-2375; Repp et al., (1995) J. Hemat. 377-382) or somatic methods (Staerz U. D. and Bevan M. J. (1986) PNAS 83; Suresh et al. (1986) Method. Enzymol. 121: 210-228) but likewise by genetic engineering techniques which allow the heterodimerization to be forced and thus facilitate the process of purification of the antibody sought (Merchand et al., 1998 Nature Biotech. 16:677-681). Examples of bispecific antibodies include those of the BiTE™ technology in which the binding domains of two antibodies with different specificity can be used and directly linked via short flexible peptides. This combines two antibodies on a short single polypeptide chain. Diabodies and scFv can be constructed without an Fc region, using only variable domains, potentially reducing the effects of anti-idiotypic reaction.

Bispecific antibodies can be constructed as entire IgG, as bispecific Fab′2, as Fab′PEG, as diabodies or else as bispecific scFv. Further, two bispecific antibodies can be linked using routine methods known in the art to form tetravalent antibodies.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against a target antigen, then a library can be made where the other arm is varied and an antibody of appropriate specificity selected. Bispecific whole antibodies may be made by alternative engineering methods as described in Ridgeway et al. (1996), Protein Eng., 9, 616-621.

Various methods are available in the art for obtaining antibodies against a target antigen. The antibodies may be monoclonal antibodies, especially of human, murine, chimeric or humanized origin, which can be obtained according to the standard methods well known to the person skilled in the art.

In general, for the preparation of monoclonal antibodies or their functional fragments, especially of murine origin, it is possible to refer to techniques which are described in particular in the manual “Antibodies” (Harlow and Lane, Antibodies: A Laboratory Manual, Cold Spring Harbor Laboratory, Cold Spring Harbor N.Y., pp. 726, 1988) or to the technique of preparation from hybridomas described by Kohler and Milstein, 1975, Nature, 256:495-497.

Monoclonal antibodies can be obtained, for example, from an animal cell immunized against A-FN, B-FN, or tenascin C or a fragment thereof containing the epitope recognized by said monoclonal antibodies, e.g. a fragment comprising or consisting of ED-A, ED-B, the A1 Domain of Tenascin C, or a peptide fragment thereof. The A-FN, B-FN, or tenascin C, or a fragment thereof, can especially be produced according to the usual working methods, by genetic recombination starting with a nucleic acid sequence contained in the cDNA sequence coding for A-FN, B-FN, or tenascin C, or fragment thereof, or by peptide synthesis starting from a sequence of amino acids comprised in the peptide sequence of the B-FN, or tenascin C, and/or a fragment thereof.

Monoclonal antibodies can, for example, be purified on an affinity column on which A-FN, B-FN, or tenascin C, or a fragment thereof containing the epitope recognized by said monoclonal antibodies, e.g. a fragment comprising or consisting of ED-A, B-FN, or tenascin C, or a peptide fragment of ED-A, B-FN, or tenascin C has previously been immobilized. Monoclonal antibodies can be purified by chromatography on protein A and/or G, followed or not followed by ion-exchange chromatography aimed at eliminating the residual protein contaminants as well as the DNA and the LPS, in itself, followed or not followed by exclusion chromatography on Sepharose gel in order to eliminate the potential aggregates due to the presence of dimers or of other multimers. The whole of these techniques may be used simultaneously or successively.

Antigen-Binding Site

This describes the part of a molecule that binds to and is complementary to all or part of the target antigen. In an antibody molecule it is referred to as the antibody antigen-binding site, and comprises the part of the antibody that binds to and is complementary to all or part of the target antigen. Where an antigen is large, an antibody may only bind to a particular part of the antigen, which part is termed an epitope. An antibody antigen-binding site may be provided by one or more antibody variable domains. An antibody antigen-binding site may comprise an antibody light chain variable region (VL) and an antibody heavy chain variable region (VH).

Isolated

This refers to the state in which specific binding members for use in the invention or nucleic acid encoding such specific binding members, will generally be in accordance with the present invention. Thus, specific binding members, VH and/or VL domains of the present invention may be provided isolated and/or purified, e.g. from their natural environment, in substantially pure or homogeneous form, or, in the case of nucleic acid, free or substantially free of nucleic acid or genes of origin other than the sequence encoding a polypeptide with the required function. Isolated members and isolated nucleic acid will be free or substantially free of material with which they are naturally associated such as other polypeptides or nucleic acids with which they are found in their natural environment, or the environment in which they are prepared (e.g. cell culture) when such preparation is by recombinant DNA technology practised in vitro or in vivo. Specific binding members and nucleic acid may be formulated with diluents or adjuvants and still for practical purposes be isolated—for example the members will normally be mixed with gelatin or other carriers if used to coat microtitre plates for use in immunoassays, or will be mixed with pharmaceutically acceptable carriers or diluents when used in diagnosis or therapy. Specific binding members may be glycosylated, either naturally or by systems of heterologous eukaryotic cells (e.g. CHO or NS0 (ECACC 85110503) cells, or they may be (for example if produced by expression in a prokaryotic cell) unglycosylated.

Heterogeneous preparations comprising antibody molecules may also be used in the invention. For example, such preparations may be mixtures of antibodies with full-length heavy chains and heavy chains lacking the C-terminal lysine, with various degrees of glycosylation and/or with derivatized amino acids, such as cyclization of an N-terminal glutamic acid to form a pyroglutamic acid residue.

One or more specific binding members for an antigen, e.g. the A-FN, the ED-A, B-FN, the ED-B, tenascin C, or the A1 domain of tenascin C may be obtained by bringing into contact a library of specific binding members according to the invention and the antigen or a fragment thereof, e.g. a fragment comprising or consisting of ED-A, ED-B, or the A1 domain of tenascin C, or a peptide fragment thereof, and selecting one or more specific binding members of the library able to bind the antigen.

An antibody library may be screened using Iterative Colony Filter Screening (ICFS). In ICFS, bacteria containing the DNA encoding several binding specificities are grown in a liquid medium and, once the stage of exponential growth has been reached, some billions of them are distributed onto a growth support consisting of a suitably pre-treated membrane filter which is incubated until completely confluent bacterial colonies appear. A second trap substrate consists of another membrane filter, pre-humidified and covered with the desired antigen.

The trap membrane filter is then placed onto a plate containing a suitable culture medium and covered with the growth filter with the surface covered with bacterial colonies pointing upwards. The sandwich thus obtained is incubated at room temperature for about 16 h. It is thus possible to obtain the expression of the genes encoding antibody fragments scFv having a spreading action, so that those fragments binding specifically with the antigen which is present on the trap membrane are trapped. The trap membrane is then treated to point out bound antibody fragments scFv with calorimetric techniques commonly used to this purpose.

The position of the colored spots on the trap filter allows one to go back to the corresponding bacterial colonies which are present on the growth membrane and produced the antibody fragments trapped. Such colonies are gathered and grown and the bacteria-a few millions of them are distributed onto a new culture membrane repeating the procedures described above. Analogous cycles are then carried out until the positive signals on the trap membrane correspond to single positive colonies, each of which represents a potential source of monoclonal antibody fragments directed against the antigen used in the selection. ICFS is described in e.g. WO0246455.

A library may also be displayed on particles or molecular complexes, e.g. replicable genetic packages such bacteriophage (e.g. T7) particles, or other in vitro display systems, each particle or molecular complex containing nucleic acid encoding the antibody VH variable domain displayed on it, and optionally also a displayed VL domain if present. Phage display is described in WO92/01047 and e.g. U.S. Pat. No. 5,969,108, U.S. Pat. No. 5,565,332, U.S. Pat. No. 5,733,743, U.S. Pat. No. 5,858,657, U.S. Pat. No. 5,871,907, U.S. Pat. No. 5,872,215, U.S. Pat. No. 5,885,793, U.S. Pat. No. 5,962,255, U.S. Pat. No. 6,140,471, U.S. Pat. No. 6,172,197, U.S. Pat. No. 6,225,447, U.S. Pat. No. 6,291,650, U.S. Pat. No. 6,492,160 and U.S. Pat. No. 6,521,404.

Following selection of specific binding members able to bind the antigen and displayed on bacteriophage or other library particles or molecular complexes, nucleic acid may be taken from a bacteriophage or other particle or molecular complex displaying a said selected specific binding member. Such nucleic acid may be used in subsequent production of a specific binding member or an antibody VH or VL variable domain by expression from nucleic acid with the sequence of nucleic acid taken from a bacteriophage or other particle or molecular complex displaying a said selected specific binding member.

Ability to bind an extra-cellular matrix component associated with neoplastic growth and/or angiogenesis, such as the A-FN, B-FN, the ED-A, or the ED-B of fibronectin, tenascin C or the A1 domain of tenascin C or other target antigen or isoform may be further tested, e.g. ability to compete with an antibody specific for the A-FN, B-FN, the ED-A, or the ED-B of fibronectin, tenascin C or the A1 domain of tenascin C, such as antibody F8, L19, or F16.

A specific binding member for use in the invention may bind an extra-cellular matrix component associated with neoplastic growth and/or angiogenesis, such as the A-FN, B-FN, the ED-A, or the ED-B of fibronectin, tenascin C or the A1 domain of tenascin C specifically. A specific binding member of the present invention may bind the A-FN and/or the ED-A of fibronectin, with the same affinity as anti-ED-A antibody F8 e.g. in diabody format, or with an affinity that is better. A specific binding member of the present invention may bind the B-FN and/or the ED-B of fibronectin, with the same affinity as anti-ED-B antibody L19 e.g. in scFv or diabody format, or with an affinity that is better. A specific binding member of the present invention may bind the Tenascin C and/or the A1 domain of tenascin C, with the same affinity as anti-ED-A antibody F16 e.g. in scFv or diabody format, or with an affinity that is better.

A specific binding member of the present invention may bind to the same epitope on A-FN and/or the ED-A of fibronectin as anti-ED-A antibody F8. A specific binding member of the present invention may bind to the same epitope on B-FN and/or the ED-B of fibronectin as anti-ED-A antibody L19. A specific binding member of the present invention may bind to the same epitope on tenascin C and/or the A1 domain of tenascin C as antibody F16.

Variants of antibody molecules disclosed herein may be produced and used in the present invention. The techniques required to make substitutions within amino acid sequences of CDRs, antibody VH or VL domains, in particular the framework regions of the VH and VL domains, and specific binding members generally are available in the art. Variant sequences may be made, with substitutions that may or may not be predicted to have a minimal or beneficial effect on activity, and tested for ability to bind A-FN and/or the ED-A of fibronectin, B-FN and/or the ED-B of fibronectin, tenascin C and/or the A1 domain of tenascin C, and/or for any other desired property.

Variable domain amino acid sequence variants of any of the VH and VL domains whose sequences are specifically disclosed herein may be employed in accordance with the present invention, as discussed. Particular variants may include one or more amino acid sequence alterations (addition, deletion, substitution and/or insertion of an amino acid residue), may be less than about 20 alterations, less than about 15 alterations, less than about 10 alterations or less than about 5 alterations, maybe 5, 4, 3, 2 or 1. Alterations may be made in one or more framework regions and/or one or more CDRs. The alterations normally do not result in loss of function, so a specific binding member comprising a thus-altered amino acid sequence may retain an ability to bind A-FN and/or the ED-A of fibronectin, B-FN and/or the ED-B of fibronectin, tenascin C and/or the A1 domain of tenascin C. For example, it may retain the same quantitative binding as a specific binding member in which the alteration is not made, e.g. as measured in an assay described herein. The specific binding member comprising a thus-altered amino acid sequence may have an improved ability to bind A A-FN and/or the ED-A of fibronectin, B-FN and/or the ED-B of fibronectin, tenascin C and/or the A1 domain of tenascin C. For example, a specific binding member that binds the ED-A isoform or ED-A of fibronectin, as referred to herein, may comprise the VH domain shown in SEQ ID NO: 18 and/or the VL domain shown in SEQ ID NO: 19 with 10 or fewer, for example, 5, 4, 3, 2 or 1 amino acid substitution within the framework region of the VH and/or VL domain. Such a specific binding member may bind the ED-A isoform or ED-A of fibronectin with the same or substantially the same, affinity as a specific binding member comprising the VH domain shown in SEQ ID NO: 18 and the VL domain shown in SEQ ID NO: 19 or may bind the ED-A isoform or ED-A of fibronectin with a higher affinity than a specific binding member comprising the VH domain shown in SEQ ID NO: 18 and the VL domain shown in SEQ ID NO: 19. A specific binding member that binds the ED-B isoform or ED-B of fibronectin, as referred to herein, may comprise the VH domain shown in SEQ ID NO: 31 and/or the VL domain shown in SEQ ID NO: 32 with 10 or fewer, for example, 5, 4, 3, 2 or 1 amino acid substitution within the framework region of the VH and/or VL domain. Such a specific binding member may bind the ED-B isoform or ED-B of fibronectin with the same or substantially the same, affinity as a specific binding member comprising the VH domain shown in SEQ ID NO: 31 and the VL domain shown in SEQ ID NO: 32 or may bind the ED-B isoform or ED-B of fibronectin with a higher affinity than a specific binding member comprising the VH domain shown in SEQ ID NO: 31 and the VL domain shown in SEQ ID NO: 32. A specific binding member that binds tenascin C or the A1 domain of tenascin C, as referred to herein, may comprise the VH domain shown in SEQ ID NO: 40 and/or the VL domain shown in SEQ ID NO: 41 with 10 or fewer, for example, 5, 4, 3, 2 or 1 amino acid substitution within the framework region of the VH and/or VL domain. Such a specific binding member may bind tenascin C or the A1 domain of tenascin C with the same or substantially the same, affinity as a specific binding member comprising the VH domain shown in SEQ ID NO: 40 and the VL domain shown in SEQ ID NO: 41 or may bind tenascin C or the A1 domain of tenascin C with a higher affinity than a specific binding member comprising the VH domain shown in SEQ ID NO: 40 and the VL domain shown in SEQ ID NO: 41.

Novel VH or VL regions carrying CDR-derived sequences for use in the invention may be generated using random mutagenesis of one or more selected VH and/or VL genes to generate mutations within the entire variable domain. In some embodiments one or two amino acid substitutions are made within an entire variable domain or set of CDRs. Another method that may be used is to direct mutagenesis to CDR regions of VH or VL genes.

As noted above, a CDR amino acid sequence substantially as set out herein may be carried as a CDR in a human antibody variable domain or a substantial portion thereof. The HCDR3 sequences substantially as set out herein represent embodiments of the present invention and for example each of these may be carried as a HCDR3 in a human heavy chain variable domain or a substantial portion thereof.

Variable domains employed in the invention may be obtained or derived from any germ-line or rearranged human variable domain, or may be a synthetic variable domain based on consensus or actual sequences of known human variable domains. A variable domain can be derived from a non-human antibody. A CDR sequence for use in the invention (e.g. CDR3) may be introduced into a repertoire of variable domains lacking a CDR (e.g. CDR3), using recombinant DNA technology. For example, Marks et al. (1992) describe methods of producing repertoires of antibody variable domains in which consensus primers directed at or adjacent to the 5′ end of the variable domain area are used in conjunction with consensus primers to the third framework region of human VH genes to provide a repertoire of VH variable domains lacking a CDR3. Marks et al. further describe how this repertoire may be combined with a CDR3 of a particular antibody. Using analogous techniques, the CDR3-derived sequences of the present invention may be shuffled with repertoires of VH or VL domains lacking a CDR3, and the shuffled complete VH or VL domains combined with a cognate VL or VH domain to provide specific binding members for use in the invention. The repertoire may then be displayed in a suitable host system such as the phage display system of WO92/01047, or any of a subsequent large body of literature, including Kay, Winter & McCafferty (1996), so that suitable specific binding members may be selected. A repertoire may consist of from anything from 10⁴ individual members upwards, for example at least 10⁵, at least 10⁶, at least 10⁷, at least 10⁸, at least 10⁹ or at least 10¹⁰ members.

Similarly, one or more, or all three CDRs may be grafted into a repertoire of VH or VL domains that are then screened for a specific binding member or specific binding members for A-FN, B-FN, the ED-A, or the ED-B of fibronectin, tenascin C or the A1 domain of tenascin C.

One or more of the HCDR1, HCDR2 and HCDR3 of antibody F8, L19, or F16, or the set of HCDRs of antibody F8, L19, or F16 may be employed, and/or one or more of the LCDR1, LCDR2 and LCDR3 of antibody F8, L19, or F16 the set of LCDRs of antibody F8, L19, or F16 may be employed.

Similarly, other VH and VL domains, sets of CDRs and sets of HCDRs and/or sets of LCDRs disclosed herein may be employed.

An extra-cellular matrix component associated with neoplastic growth and/or angiogenesis, such as the A-FN, B-FN, the ED-A, or the ED-B of fibronectin, tenascin C or the A1 domain of tenascin C may be used in a screen for specific binding members, e.g. antibody molecules, for use in the invention. The screen may a screen of a repertoire as disclosed elsewhere herein.

A substantial portion of an immunoglobulin variable domain may comprise at least the three CDR regions, together with their intervening framework regions. The portion may also include at least about 50% of either or both of the first and fourth framework regions, the 50% being the C-terminal 50% of the first framework region and the N-terminal 50% of the fourth framework region. Additional residues at the N-terminal or C-terminal end of the substantial part of the variable domain may be those not normally associated with naturally occurring variable domain regions. For example, construction of specific binding members of the present invention made by recombinant DNA techniques may result in the introduction of N- or C-terminal residues encoded by linkers introduced to facilitate cloning or other manipulation steps. Other manipulation steps include the introduction of linkers to join variable domains disclosed elsewhere herein to further protein sequences including antibody constant regions, other variable domains (for example in the production of diabodies) or detectable/functional labels as discussed in more detail elsewhere herein.

Although specific binding members may comprise a pair of VH and VL domains, single binding domains based on either VH or VL domain sequences may also be used in the invention. It is known that single immunoglobulin domains, especially VH domains, are capable of binding target antigens in a specific manner. For example, see the discussion of dAbs above.

In the case of either of the single binding domains, these domains may be used to screen for complementary domains capable of forming a two-domain specific binding member able to bind an extra-cellular matrix component associated with neoplastic growth and/or angiogenesis, such as A-FN, B-FN, the ED-A, or the ED-B of fibronectin, tenascin C or the A1 domain of tenascin C. This may be achieved by phage display screening methods using the so-called hierarchical dual combinatorial approach as disclosed in WO92/01047, in which an individual colony containing either an H or L chain clone is used to infect a complete library of clones encoding the other chain (L or H) and the resulting two-chain specific binding member is selected in accordance with phage display techniques such as those described in that reference. This technique is also disclosed in Marks 1992.

Specific binding members for use in the present invention may further comprise antibody constant regions or parts thereof, e.g. human antibody constant regions or parts thereof. For example, a VL domain may be attached at its C-terminal end to antibody light chain constant domains including human C kappa or C lambda chains, e.g. C lambda. Similarly, a specific binding member based on a VH domain may be attached at its C-terminal end to all or part (e.g. a CH1 domain) of an immunoglobulin heavy chain derived from any antibody isotype, e.g. IgG, IgA, IgE and IgM and any of the isotype sub-classes, particularly IgG1 and IgG4. Any synthetic or other constant region variant that has these properties and stabilizes variable regions is also useful in embodiments of the present invention.

In the context of the present invention, a specific binding member (e.g. antibody), as described herein, forms part of a conjugate with IL4. The IL4 is preferably human IL4. The specific binding member preferably comprises an scFv or is a diabody.

The specific binding member and IL4 may be connected to each other directly, for example through any suitable chemical bond, or through a linker, for example a peptide linker. Where the specific binding member is linked to IL4 by means of a peptide linker, the conjugate may be fusion protein.

The chemical bond may be, for example, a covalent or ionic bond. Examples of covalent bonds include peptide bonds (amide bonds) and disulphide bonds. The specific binding member and IL4 may be covalently linked, for example by peptide bonds (amide bonds). Thus, the specific binding member, in particular an scFv portion of a specific binding member, and IL4 may be produces as a fusion protein. Where the specific binding member is a two-chain or multi-chain molecule (e.g. a diabody). IL4 may be conjugated as a fusion polypeptide with one or more polypeptide chains in the specific binding member.

The peptide linker connecting the specific binding member and IL4 may be a flexible peptide linker. Suitable examples of peptide linker sequences are known in the art. The linker may be 10-20 amino acids, preferably 15-20 amino acids in length. Most preferably, the linker is 15 amino acids in length. Most preferably, the linker has the sequence SSSSGSSSSGSSSSG (SEQ ID NO: 24).

Other means for conjugation include chemical conjugation, especially cross-linking using a bifunctional reagent (e.g. employing DOUBLE-REAGENTS™ Cross-linking Reagents Selection Guide, Pierce).

Also provided is an isolated nucleic acid molecule encoding a conjugate according to the present invention. Nucleic acid molecules may comprise DNA and/or RNA and may be partially or wholly synthetic. Reference to a nucleotide sequence as set out herein encompasses a DNA molecule with the specified sequence, and encompasses a RNA molecule with the specified sequence in which U is substituted for T, unless context requires otherwise.

Further provided are constructs in the form of plasmids, vectors, transcription or expression cassettes which comprise such nucleic acids. Suitable vectors can be chosen or constructed, containing appropriate regulatory sequences, including promoter sequences, terminator sequences, polyadenylation sequences, enhancer sequences, marker genes and other sequences as appropriate. Vectors may be plasmids e.g. phagemid, or viral e.g. 'phage, as appropriate. For further details see, for example, Sambrook & Russell (2001) Molecular Cloning: a Laboratory Manual: 3rd edition, Cold Spring Harbor Laboratory Press. Many known techniques and protocols for manipulation of nucleic acid, for example in the preparation of nucleic acid constructs, mutagenesis, sequencing, introduction of DNA into cells and gene expression, and analysis of proteins, are described in detail in Ausubel et al. (1999) 4^{th} eds., Short Protocols in Molecular Biology: A Compendium of Methods from Current Protocols in Molecular Biology, John Wiley & Sons.

A recombinant host cell that comprises one or more constructs as described above is also provided. Suitable host cells include bacteria, mammalian cells, plant cells, filamentous fungi, yeast and baculovirus systems and transgenic plants and animals.

A conjugate according to the present invention may be produced using such a recombinant host cell. The production method may comprise expressing a nucleic acid or construct as described above. Expression may conveniently be achieved by culturing the recombinant host cell under appropriate conditions for production of the conjugate. Following production the conjugate may be isolated and/or purified using any suitable technique, and then used as appropriate. The conjugate may be formulated into a composition including at least one additional component, such as a pharmaceutically acceptable excipient.

Systems for cloning and expression of a polypeptide in a variety of different host cells are well known. The expression of antibodies, including conjugates thereof, in prokaryotic cells is well established in the art. For a review, see for example Plückthun (1991), Bio/Technology 9: 545-551. A common bacterial host is E. coli.

Expression in eukaryotic cells in culture is also available to those skilled in the art as an option for production of conjugates for example Chadd et al. (2001), Current Opinion in Biotechnology 12: 188-194); Andersen et al. (2002) Current Opinion in Biotechnology 13: 117; Larrick & Thomas (2001) Current Opinion in Biotechnology 12:411-418. Mammalian cell lines available in the art for expression of a heterologous polypeptide include Chinese hamster ovary (CHO) cells, HeLa cells, baby hamster kidney cells, NS0 mouse melanoma cells, YB2/0 rat myeloma cells, human embryonic kidney cells, human embryonic retina cells and many others.

A method comprising introducing a nucleic acid or construct disclosed herein into a host cell is also described. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. Introducing nucleic acid in the host cell, in particular a eukaryotic cell may use a viral or a plasmid based system. The plasmid system may be maintained episomally or may be incorporated into the host cell or into an artificial chromosome. Incorporation may be either by random or targeted integration of one or more copies at single or multiple loci. For bacterial cells, suitable techniques may include calcium chloride transformation, electroporation and transfection using bacteriophage.

The nucleic acid may or construct be integrated into the genome (e.g. chromosome) of the host cell. Integration may be promoted by inclusion of sequences that promote recombination with the genome, in accordance with standard techniques.

The conjugates of the present invention are designed to be used in methods of treatment of patients, preferably human patients. Conjugates of the present invention may be used in the treatment of a disease/disorder, such as cancer and/or autoimmune diseases, such as rheumatoid arthritis (RA), multiple sclerosis (MS), inflammatory bowel disease (IBD), psoriasis, psoriatic arthritis, endometriosis, Behçet's disease and periodontitis. Other diseases which may be treated or prevented using the conjugates of the invention include autoimmune insulitis and diabetes, in particular autoimmune diabetes. Polymorbid patients, i.e. patients suffering from more than one of these disease may also be treated using the conjugates of the present invention. Preferably, the conjugates of the present invention are used to treat cancer and/or RA. Most preferably, the conjugates of the present invention are used to treat RA.

Accordingly, the invention provides methods of treatment comprising administration of a conjugate according to the present invention, pharmaceutical compositions comprising such conjugates, and use of such a conjugates in the manufacture of a medicament for administration, for example in a method of making a medicament or pharmaceutical composition comprising formulating the conjugate with a pharmaceutically acceptable excipient. Pharmaceutically acceptable vehicles are well known and will be adapted by the person skilled in the art as a function of the nature and of the mode of administration of the active compound(s) chosen.

Conjugates according to the present invention will usually be administered in the form of a pharmaceutical composition, which may comprise at least one component in addition to the specific binding member. Thus, pharmaceutical compositions described herein, and for use in accordance with the present invention, may comprise, in addition to active ingredient, a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration, which may be by injection, e.g. intravenous or subcutaneous. Preferably, the conjugate of the present invention is administered intravenously, in particular where the disease to be treated or prevented is cancer, MS, IBD, psoriasis, psoriatic arthritis, periodontitis, endometriosis, Behçet's disease, insulitis or diabetes. Where the treatment concerns RA, the conjugate may administered subcutaneously.

Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be employed, as required. Many methods for the preparation of pharmaceutical formulations are known to those skilled in the art. See e.g. Robinson ed., Sustained and Controlled Release Drug Delivery Systems, Marcel Dekker, Inc., New York, 1978.

A composition comprising a conjugate according to the present invention may be administered alone or in combination with other treatments, concurrently or sequentially or as a combined preparation with another therapeutic agent or agents, dependent upon the condition to be treated.

For example, a specific binding member for use in the invention may be used in combination with an existing therapeutic agent for the disease to be treated.

Where the conjugate of the invention is used in the treatment of cancer, or delivery of IL4 to the tumour neovasculature, the conjugate of the invention is preferably administered in combination with a second conjugate, wherein the second conjugate comprises IL12 or IL2 and a binding member which binds an extra-cellular matrix component associated with neoplastic growth and/or angiogenesis.

Where the conjugate of the invention is used in treatment of an inflammatory autoimmune disease, such as RA, or delivery of IL4 to the sites of an inflammatory autoimmune disease, the conjugate of the invention is preferably administered in combination with glucocorticoid. The glucocorticoid is preferably dexamethasone.

A conjugate according to the invention and one or more additional medicinal components, such as a second conjugate or glucocorticoid as described above and elsewhere herein, may be used in the manufacture of a medicament. The medicament may be for separate or combined administration to an individual, and accordingly may comprise the conjugate and the additional component as a combined preparation or as separate preparations. Separate preparations may be used to facilitate separate and sequential or simultaneous administration, and allow administration of the components by different routes.

In accordance with the present invention, compositions provided may be administered to mammals, preferably humans. Administration may be in a “therapeutically effective amount”, this being sufficient to show benefit to a patient. Such benefit may be at least amelioration of at least one symptom. Thus “treatment” of a specified disease refers to amelioration of at least one symptom. The actual amount administered, and rate and time-course of administration, will depend on the nature and severity of what is being treated, the particular patient being treated, the clinical condition of the individual patient, the cause of the disorder, the site of delivery of the composition, the type of conjugate, the method of administration, the scheduling of administration and other factors known to medical practitioners. Prescription of treatment, e.g. decisions on dosage etc., is within the responsibility of general practitioners and other medical doctors, and may depend on the severity of the symptoms and/or progression of a disease being treated. Appropriate doses of antibody are well known in the art (Ledermann et al. (1991) Int. J. Cancer 47: 659-664; and Bagshawe et al. (1991) Antibody, Immunoconjugates and Radiopharmaceuticals 4: 915-922). Specific dosages indicated herein, or in the Physician's Desk Reference (2003) as appropriate for the type of medicament being administered, may be used. A therapeutically effective amount or suitable dose of a conjugate for use in the invention can be determined by comparing its in vitro activity and in vivo activity in an animal model. Methods for extrapolation of effective dosages in mice and other test animals to humans are known. The precise dose will depend upon a number of factors, including whether the antibody is for diagnosis, prevention or for treatment, the size and location of the area to be treated, the precise nature of the conjugate. A typical conjugate dose will be in the range 100 μg to 1 g for systemic applications. An initial higher loading dose, followed by one or more lower doses, may be administered. This is a dose for a single treatment of an adult patient, which may be proportionally adjusted for children and infants, and also adjusted according to conjugate format in proportion to molecular weight. Treatments may be repeated at daily, twice-weekly, weekly or monthly intervals, at the discretion of the physician. Treatments may be every two to four weeks for subcutaneous administration and every four to eight weeks for intravenous administration. In some embodiments of the present invention, treatment is periodic, and the period between administrations is about two weeks or more, e.g. about three weeks or more, about four weeks or more, or about once a month. In other embodiments of the invention, treatment may be given before, and/or after surgery, and may be administered or applied directly at the anatomical site of surgical treatment.

Fibronectin

Fibronectin is an antigen subject to alternative splicing, and a number of alternative isoforms of fibronectin are known, including alternatively spliced isoforms A-FN and B-FN, comprising domains ED-A or ED-B respectively, which are known markers of angiogenesis. A specific binding member, as referred to herein, may selectively bind to isoforms of fibronectin selectively expressed in the neovasculature. A specific binding member may bind fibronectin isoform A-FN, e.g. it may bind domain ED-A (extra domain A). A specific binding member may bind ED-B (extra domain B).

Fibronectin Extra Domain-A (EDA or ED-A) is also known as ED, extra type III repeat A (EIIIA) or EDI. The sequence of human ED-A has been published by Kornblihtt et al. (1984), Nucleic Acids Res. 12, 5853-5868 and Paolella et al . (1988), Nucleic Acids Res. 16, 3545-3557. The sequence of human ED-A is also available on the SwissProt database as amino acids 1631-1720 (Fibronectin type-III 12; extra domain 2) of the amino acid sequence deposited under accession number P02751. The sequence of mouse ED-A is available on the SwissProt database as amino acids 1721-1810 (Fibronectin type-III 13; extra domain 2) of the amino acid sequence deposited under accession number P11276.

The ED-A isoform of fibronectin (A-FN) contains the Extra Domain-A (ED-A). The sequence of the human A-FN can be deduced from the corresponding human fibronectin precursor sequence which is available on the SwissProt database under accession number P02751. The sequence of the mouse A-FN can be deduced from the corresponding mouse fibronectin precursor sequence which is available on the SwissProt database under accession number P11276. The A-FN may be the human ED-A isoform of fibronectin. The ED-A may be the Extra Domain-A of human fibronectin.

ED-A is a 90 amino acid sequence which is inserted into fibronectin (FN) by alternative splicing and is located between domain 11 and 12 of FN (Borsi et al. (1987), J. Cell. Biol., 104, 595-600). ED-A is mainly absent in the plasma form of FN but is abundant during embryogenesis, tissue remodeling, fibrosis, cardiac transplantation and solid tumour growth.

Fibronectin isoform B-FN is one of the best known markers angiogenesis (U.S. Ser. No. 10/382,107, WO01/62298). An extra domain “ED-B” of 91 amino acids is found in the B-FN isoform and is identical in mouse, rat, rabbit, dog and man. B-FN accumulates around neovascular structures in aggressive tumours and other tissues undergoing angiogenesis, such as the endometrium in the proliferative phase and some ocular structures in pathological conditions, but is otherwise undetectable in normal adult tissues.

Tenascin C

Tenascin-C is a large hexameric glycoprotein of the extracellular matrix which modulates cellular adhesion. It is involved in processes such as cell proliferation and cell migration and is associated with changes in tissue architecture as occurring during morphogenesis and embryogenesis as well as under tumourigenesis or angiogenesis. Several isoforms of tenascin-C can be generated as a result of alternative splicing which may lead to the inclusion of (multiple) domains in the central part of this protein, ranging from domain A1 to domain D (Borsi L et al Int J Cancer 1992; 52:688-692, Carnemolla B et al. Eur J Biochem 1992; 205:561-567, WO2006/050834). A specific binding member, as referred to herein, may bind tenascin-C. A specific binding member may bind tenascin-C domain A1.

Cancer

Cancer, as referred to herein, may be a cancer which expresses, or has been shown to express, the ED-A isoform of fibronectin, the ED-B isoform of fibronectin and/or alternatively spliced tenascin C. Preferably the cancer expresses the ED-A isoform of fibronectin. For example, the cancer may be any type of solid or non-solid cancer or malignant lymphoma and especially germ cell cancer (such as teratocarcinoma), liver cancer, lymphoma (such as Hodgkin's or non-Hodgkin's lymphoma), leukaemia (e.g. acute myeloid leukaemia), sarcomas, skin cancer, melanoma, sarcoma, bladder cancer, breast cancer, uterine cancer, ovarian cancer, prostate cancer, lung cancer, colorectal cancer, cervical cancer, head and neck cancer, oesophageal cancer, pancreatic cancer, renal cancer, stomach cancer and cerebral cancer. Cancers may be familial or sporadic. Cancers may be metastatic or non-metastatic. Preferably, the cancer is a cancer selected from the group consisting of germ cell cancer (such as teratocarcinoma); colorectal cancer; Hodgkin's or non-Hodgkin's lymphoma; melanoma; pancreatic cancer; soft tissue sarcoma; or renal cell carcinoma.

Inflammatory Autoimmune Diseases

An inflammatory autoimmune disease, as referred to herein, may be an inflammatory autoimmune disease which is characterized by, or has been shown to be characterized by, expression of the ED-A isoform of fibronectin, the ED-B isoform of fibronectin and/or tenascin C. The conjugate used in the treatment of an inflammatory autoimmune disease, or delivery of IL4 to sites of inflammatory autoimmune disease in a patient, may be selected based on the expression of the ED-A isoform of fibronectin, ED-B isoform of fibronectin and tenascin C in said inflammatory autoimmune disease. Preferably, the inflammatory autoimmune disease is selected from the group consisting of: rheumatoid arthritis (RA), multiple sclerosis (MS), endometriosis, autoimmune diabetes (such as diabetes mellitus type 1), inflammatory bowel disease (IBD), psoriasis, psoriatic arthritis, and periodontitis. More preferably, the autoimmune disease is selected from the group consisting of: rheumatoid arthritis (RA), multiple sclerosis (MS), endometriosis, autoimmune diabetes (such as diabetes mellitus type 1), and psoriasis.

Rheumatoid Arthritis

Rheumatoid arthritis (RA) is an autoimmune disease that may result in a chronic, systemic inflammatory disorder that may affect many tissues and organs, but principally attacks flexible (synovial) joints.

Psoriasis

Psoriasis is an autoimmune disease that may result in a chronic systemic inflammatory disorder that may affect any part of the body but is most commonly found on the elbows, knees, lower back and scalp. Psoriasis may result in red, flaky patches of skin covered with silvery scales.

Psoriatic Arthritis

Psoriatic arthritis is an autoimmune disease which causes inflammation and pain in the joints, although other parts of the body may also be affected. Psoriatic arthritis is a type of inflammatory arthritis and is often associated with psoriasis.

Endometriosis

Endometriosis is a gynecological disease in which cells from the lining of the uterus (endometrium) appear and flourish outside the uterine cavity. Endometriosis causes pain and infertility.

Behçet's Disease

Behçet's disease is an immune-mediated small-vessel systemic vasculitis-that often presents with mucous membrane ulceration and ocular problems.

Multiple Sclerosis

Multiple sclerosis is an inflammatory disease in which the fatty myelin sheaths around the axons of the brain and spinal cord are damaged, leading to demyelination and scarring. “Multiple sclerosis”, as referred to herein, may refer to relapsing remitting, secondary progressive, primary progressive, and/or progressive relapsing, multiple sclerosis.

Endometriosis

Endometriosis is a condition in which cells from the endometrium grow outside the uterine cavity. Symptoms of endometriosis include pelvic pain and fertility problems. Endometriosis, as referred to herein, may be Stage I, Stage II, Stage III, and/or Stage IV endometriosis according to the Revised Classification of the American Society of Reproductive Medicine, 1996, Fertility and Sterility 67 (5): 817-21.

Autoimmune Insulitis

Autoimmune Insulitis refers to a lymphocytic infiltration of the the islets of Langerhans of the pancreas. Autoimmune Insulitis is frequently associated with new-onset type 1 diabetes mellitus.

Autoimmune Diabetes

Autoimmune diabetes is a form of diabetes mellitus that results from autoimmune destruction of the insulin-producing islets of Langerhans of the pancreas. Autoimmune disease diabetes can occur in both adults and children. Autoimmune diabetes, as referred to herein, is preferably diabetes mellitus type 1.

Inflammatory Bowel Disease (IBD)

Inflammatory Bowel Disease is a group of inflammatory conditions that affect the colon and small intestine. The major types of IBD are Crohn's disease (CD) and ulcerative colitis (UC), while other types of IBD include collagenous colitis, lymphocytic colitis, ischaemic colitis, diversion colitis, Behçet's disease and indeterminate colitis. CD can affect any part of the gastrointestinal tract, whereas UC is typically restricted to the colon and rectum.

IBD, as referred to herein, may be CD, UC, collagenous colitis, lymphocytic colitis, ischaemic colitis, diversion colitis, Behçet's disease or indeterminate colitis. In particular, the terms CD, UC, collagenous colitis, lymphocytic colitis, ischaemic colitis, diversion colitis, Behçet's disease and indeterminate colitis, as used herein, may refer to active CD, active UC, active collagenous colitis, active lymphocytic colitis, active ischaemic colitis, active diversion colitis, and active indeterminate colitis, respectively. In one embodiment, the IBD may be CD or UC.

Further aspects and embodiments of the invention will be apparent to those skilled in the art given the present disclosure including the following experimental exemplification.

All documents mentioned in this specification are incorporated herein by reference in their entirety. “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above.

### EXAMPLES

The present examples describe the construction of antibody-IL4 conjugates and demonstrate the suitability of such conjugates for treating rheumatoid arthritis and cancer.

Examples Relating to the Preparation and In Vitro Characterization of Antibody-IL4 Conjugates

### Example 1

### Cloning, Expression and In Vitro Characterization of F8-IL4

A conjugate, F8-IL4, containing the antibody F8 (Villa et al., 2008) (specific to the EDA domain of fibronectin, a marker or tumour angiogenesis) in a stable non-covalent homodimeric diabody format (i.e., scFv fragment with a 5-amino acid linker between VH and VL domains), sequentially fused at the C-terminus to murine interleukin 4 (gene from Source BioScience) via a flexible 15 amino acid linker (SEQ ID NO: 24), was prepared. The gene encoding the F8 antibody (SEQ ID NO: 9) and the gene encoding murine IL4 (SEQ ID NO: 51) were PCR amplified and PCR assembled using standard procedures as described previously (Pasche et al., 2011) to prepare F8-IL4 (SEQ ID NO: 21). The product was ligated into the mammalian expression vector pcDNA3.1(+) (Invitrogen) by a HindIII/NotI restriction site.

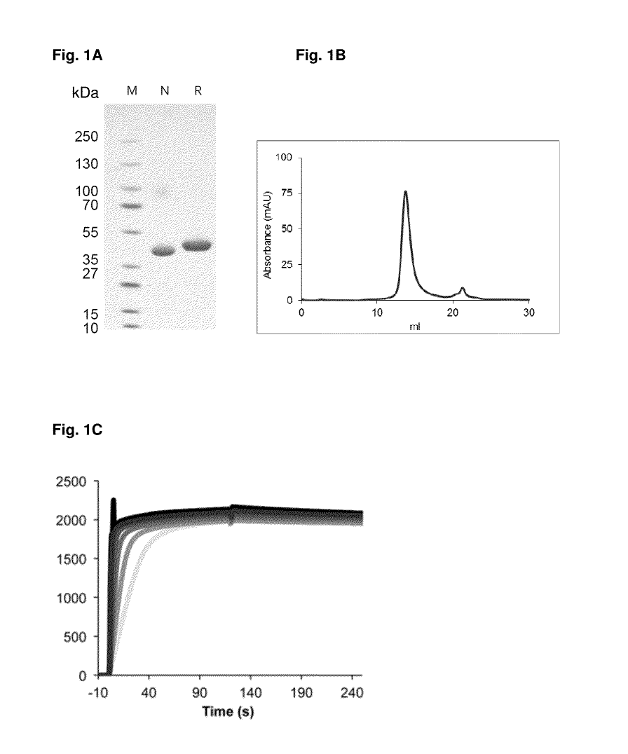

The fusion protein was expressed in stably transfected CHO cells (Invitrogen) grown according to the supplier's protocol and purified to homogeneity by protein A chromatography, as documented by SDS-PAGE analysis and size-exclusion chromatography (Superdex200 10/300GL, GE Healthcare) (see FIGS. 1A and B). The products retained a high-affinity for the cognate antigen, as revealed by surface plasmon analysis (BIAcore) on an EDA antigen-coated sensor chip (FIG. 1C).

Examples Relating to the Treatment of RA using Antibody-IL4 Conjugates

### Example 2

### Preparation and In Vitro Characterization of TNFR-Fc

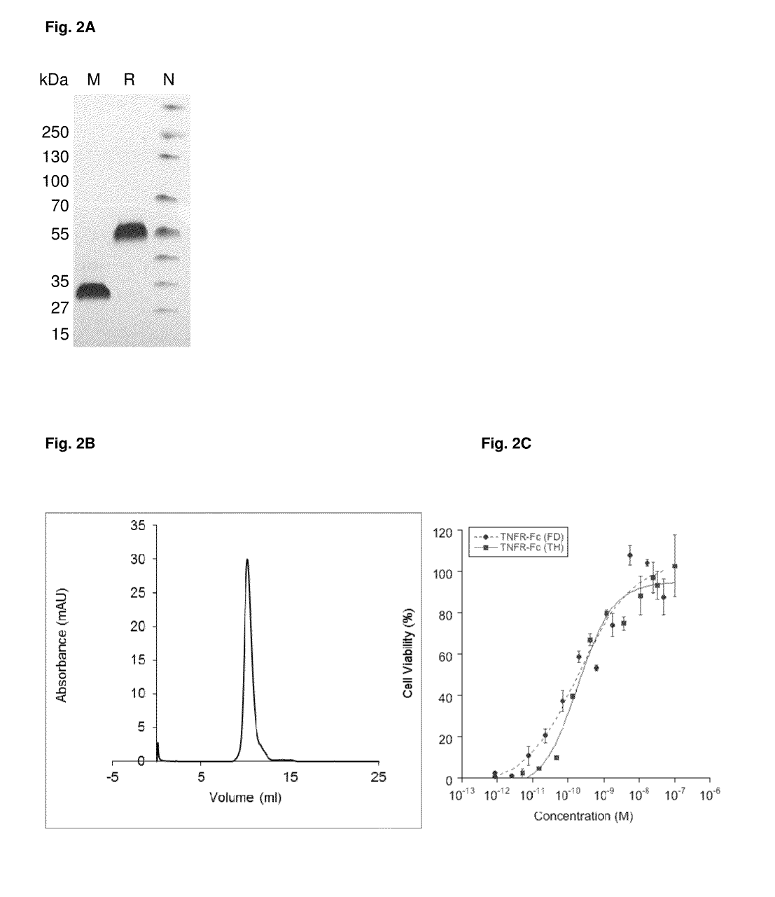

The murine fusion protein muTNFR-Fc (extracellular part of the murine p75-TNF receptor appended at the N-terminus of a murine IgG1 Fc portion, containing the hinge region) was expressed by stable transfection in CHO cells, according to the supplier's protocol. The fusion protein was purified from the culture supernatant by protein A chromatography, yielding a preparation which was pure in SDS-PAGE analysis and size exclusion chromatography (see FIGS. 2A and B). The biological activity of muTNFR-Fc was tested by inhibition of TNF-mediated killing of LM fibroblasts (see FIG. 2C). TNF inhibitors such as Enbrel™ and Humeira™ represent the standard of care in rheumatoid arthritis patients. muTNFR-Fc is a murine version of a TNF inhibitor for use in mouse models of the disease.

### Example 3

### Characterization of Therapeutic Potential of F8-mIL4 in an Aggressive Model of Collagen Induced Arthritis in the Mouse

Male DBA/1J mice were obtained from Janvier (Le Genest-St-Isle, France). 8 week old male DBA/1J mice were immunized by subcutaneous injection at the base of the tail with 0.05 mL of an emulsion of bovine type II collagen emulsified in Completes Freund's Adjuvant (CFA) with a concentration of 0.645 mg/mL collagen (Hooke Laboratories, Lawrence, USA). Three weeks later, a booster injection of 0.04 mL of 0.98 mg/ml bovine collagen/CFA was given. After the booster injection mice were inspected daily and disease was monitored by assignation of a clinical score to each score (0=normal, 1=one toe inflamed and swollen, 2=more than one toe, but not entire paw, inflamed and swollen or mild swelling of entire paw, 3=entire paw inflamed and swollen, 4=very inflamed and swollen paw; adapted from Hooke Laboratories). A maximum score of 16 can be reached. In addition, swelling of affected paws was measured daily with a caliper under isoflurane anesthesia. Paw thickness is expressed as the mean of all four paws of each animal. Animals showing signs of joint inflammation with a total score of 1 to 3 were included in the therapy experiments. When the joint inflammation was too high at day one (more than one paw, score>3) mice were not included in the therapy experiments. Experiments were performed under a project license granted by the Veterinaeramt des Kantons Zuerich, Switzerland (208/2010).

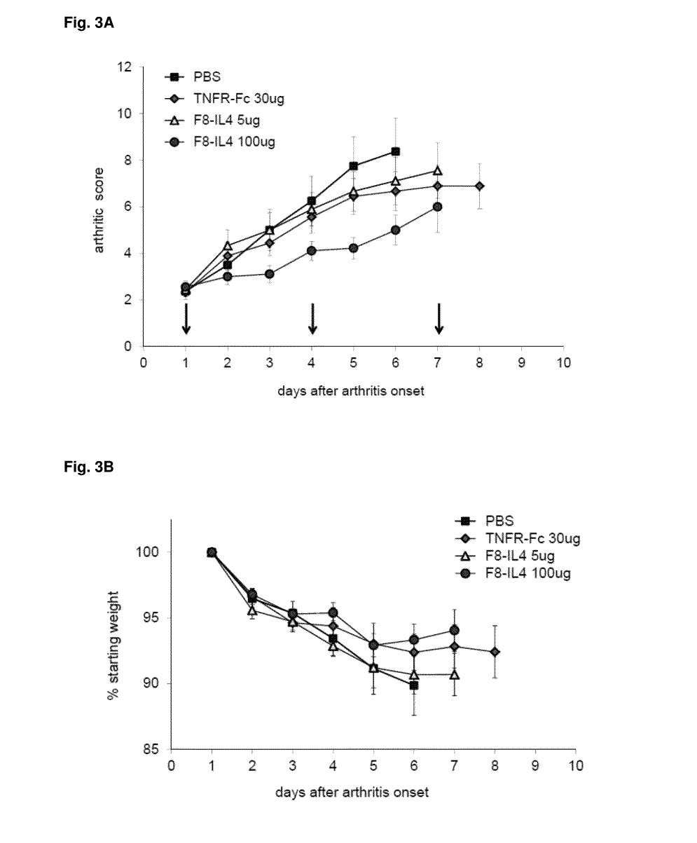

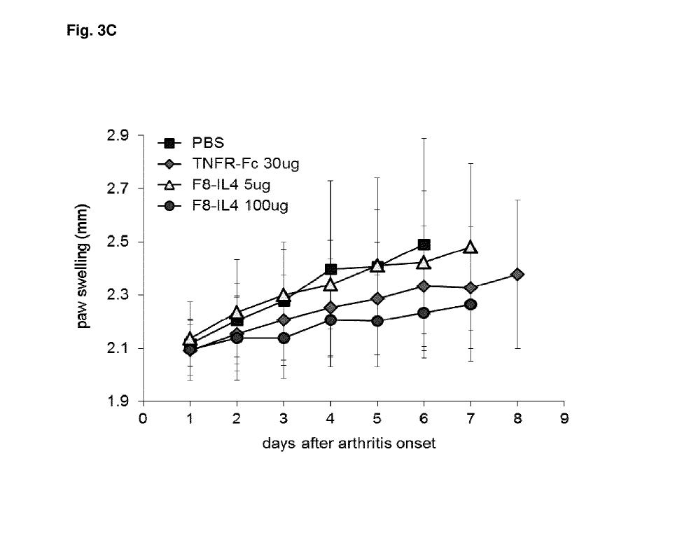

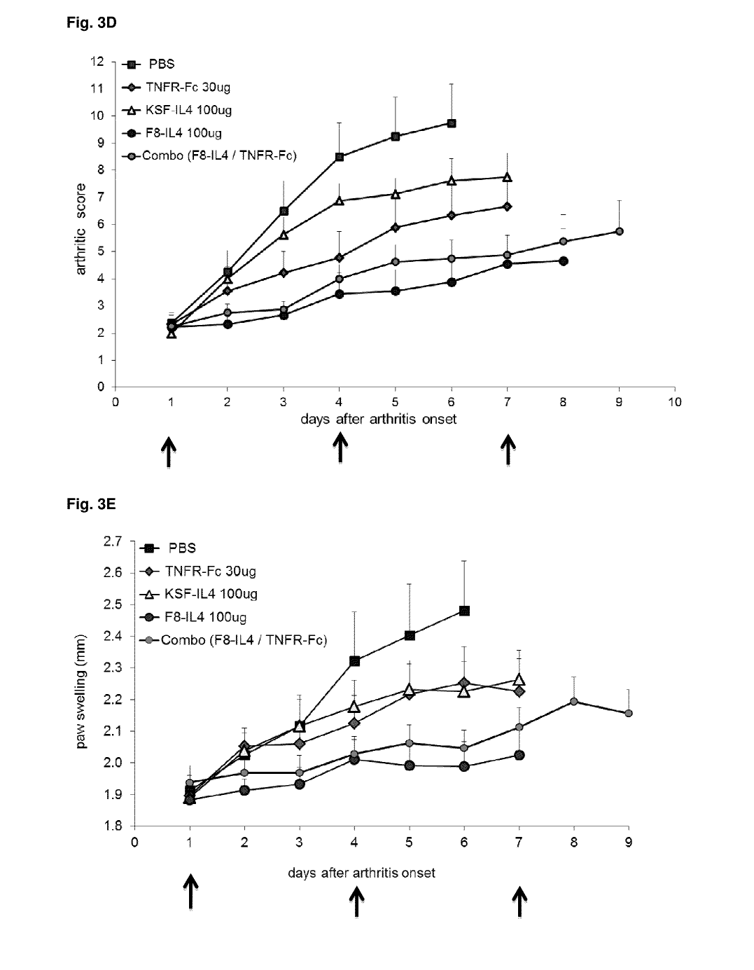

Mice with a new clinical score of 1 to 3 were randomly assigned to a treatment or control group and therapy was started (=day 1). Mice received intravenous injections of muTNFR-Fc (30 μg), F8-IL4 (5 μg), F8-IL4 (100 μg) or PBS (buffer vehicle, negative control) on days 1, 4 and 7. Mice were analyzed per group (n≧9) daily and the arthritic sore, the thickness of inflamed paws and weight was monitored. The results of these experiments are shown in FIG. 3A-C. Mice were sacrificed in accordance with local regulations due to arthritic score (>6 for more than 4 days) and weight loss (>15%). TNFR-Fc and F8-IL4 were administered in differing amounts as they have different effector functions and toxicity potential. The dose for TNFR-Fc was determined by calculations from the human ENBREL dose and the approximate local TNF concentration compared with the bioactivity of the fusion protein. The 30 μg dose for TNFR-Fc was confirmed in a previous experiment with a more moderate mouse model of RA, where a stronger disease modulating effect was observed at this dose. The 30 μg dose of TNFR-Fc administered to the mice in this experiment therefore represents an appropriate comparison for determining the efficacy of the F8-IL4 conjugate in treating RA.

F8-IL4 in the high dose schedule (100 μg/injection) exhibited a disease-modulating effect which was at least as potent as the murine version of Enbrel (TNFR-Fc) in this mouse model of aggressive arthritis. Mice receiving F8-IL4 at a dose of 100 μg/injection also lost less weight than mice receiving only the buffer vehicle (PBS). This indicates that F8-IL4 therapy (100 μg/injection) was well tolerated and mice were in a better general state of health. Mice treated with 100 μg of F8-IL4 also exhibited less severe paw swelling than mice receiving only the buffer vehicle or low amounts of F8-IL4. The reduction in paw swelling was at least equivalent to that observed in mice treated with the murine version of ENBREL. The mouse model used in these experiments was a model of aggressive RA, which results in fast disease progression and early endpoints for the analysis, as mice had to be sacrificed in accordance with local regulations where the arthritic score was >6 for more than 4 days and weight loss was >15%. This explains the convergence of data points for the differently treated mice at later time points, in particular 7 days after RA onset, in FIG. 3A-C. The convergence of the data points at later time points does not affect the validity of the clear differences in disease progression observed at earlier time points between the differently treated mice, as summarized above.

### Example 3a

### Comparison of the Therapeutic Potential of Targeted and Untargeted IL4 in an Aggressive Model of Collagen Induced Arthritis in the Mouse

Male DBA/1J mice were obtained from Janvier (Le Genest-St-Isle, France). 8 week old male DBA/1J mice were immunized by subcutaneous injection at the base of the tail with 0.05 ml of an emulsion of bovine type II collagen emulsified in Completes Freund's Adjuvant (CFA) with a concentration of 0.645 mg/ml collagen (Hooke Laboratories, Lawrence, USA). Three weeks later, a booster injection of 0.04 ml of 0.645 mg/ml bovine collagen/CFA was given. After the booster injection mice were inspected daily and disease was monitored by assignation of a clinical score to each mouse (0=normal, 1=one toe inflamed and swollen, 2=more than one toe, but not entire paw, inflamed and swollen or mild swelling of entire paw, 3=entire paw inflamed and swollen, 4=very inflamed and swollen paw; adapted from Hooke Laboratories). A maximum score of 16 can be reached. In addition, swelling of affected paws was measured daily with a caliper under isoflurane anaesthesia. Paw thickness is expressed as the mean of all four paws of each animal.

Mice with a new clinical score of 1 to 4 were randomly assigned to a treatment or control group and therapy was started (=day 1). 9 mice received intravenous (i.v.) injections of F8-IL4 (100 μg) (SEQ ID NO: 21), 8 mice received intravenous (i.v.) injections of KSF-IL4 (100 μg) (SEQ ID NO: 44), 9 mice received intravenous (i.v.) injections of TNFR-Fc (30 μg) (SEQ ID NO: 46), 9 mice received intravenous (i.v.) injections of TNFR-Fc (30 μg) (SEQ ID NO: 46) and F8-IL4 (100 μg) (SEQ ID NO: 21), and 8 mice received PBS. Injections were administered on day 1, 3 and 7.

Mice were analyzed per group (n≧8) daily and the arthritic sore, the thickness of inflamed paws and weight was monitored. Paw swelling of was measured daily and paw thickness is expressed as the mean of thickness of all four paws of each animal (±SEM). The results of these experiments (arthritic score and paw thickness) are shown in FIGS. 3D and E. Mice were sacrificed in accordance with local regulations due to arthritic score (>6 for more than 4 days) and weight loss (>15%). This resulted in the early termination of some of the treatment schedules.

FIGS. 3D and E compare the effect of F8-IL4 to KSF-IL4 at the dose found to give the highest activity in FIG. 3A-C (100 μg/injection). The statistically significant superior activity of F8-IL4 compared to KSF-IL4 in reducing arthritic score and paw swelling indicates that targeting is important for the therapeutic efficacy. No synergistic effect was observed when F8-IL4 and TNFR-Fc were administered to the mice simultaneously.

All groups in this therapy showed a slightly better performance to the FIG. 3A-C, as the booster immunization in the experiment comparing the 2 doses of F8-IL4 was much stronger as the kits were higher concentrated. Therefore mice developed an even more aggressive form of arthritis.

### Example 4

### Treatment of Rheumatoid Arthritis in an Aggressive Model of Collagen Induced Arthritis in the Mouse using F8-Murine IL4 in Combination with Dexamethasone or L19-IL10

Male DBA/1J mice were obtained from Janvier (Le Genest-St-Isle, France). 8 week old male DBA/1J mice were immunized by subcutaneous injection at the base of the tail with 0.05 ml of an emulsion of bovine type II collagen emulsified in Completes Freund's Adjuvant (CFA) with a concentration of 0.645 mg/ml collagen (Hooke Laboratories, Lawrence, USA). Three weeks later, a booster injection of 0.04 ml of 0.645 mg/ml bovine collagen/CFA was given. After the booster injection mice were inspected daily and disease was monitored by assignation of a clinical score to each mouse (0=normal, 1=one toe inflamed and swollen, 2=more than one toe, but not entire paw, inflamed and swollen or mild swelling of entire paw, 3=entire paw inflamed and swollen, 4=very inflamed and swollen paw; adapted from Hooke Laboratories). A maximum score of 16 can be reached. In addition, swelling of affected paws was measured daily with a calliper under isoflurane anaesthesia. Paw thickness is expressed as the mean of all four paws of each animal.

Mice with a new clinical score of 1 to 4 were randomly assigned to a treatment or control group and therapy was started (=day 1). 10 mice received subcutaneous (s.c.) injections of F8-IL4 (100 μg), 9 mice received intravenous (i.v.) injections of F8-IL4 (100 μg), 8 mice received dexamethasone (100 μg), 9 mice received F8-IL4 and dexamethasone (100 μg of each), 10 mice received L19-IL10 (200 μg), 9 mice received F8-IL4 and L19-IL10 (100 μg and 200 μg, respectively), 9 mice received PBS. F8-IL4 was injected either intravenously (i.v.) or subcutaneously (s.c.) on days 1, 3 and 7 in the monotherapy groups. F8-IL4 was always administered intravenously (i.v.) in the combination groups (i.e. with dexamethasone or with L19-IL10) on days 1, 3 and 7. L19-IL10 was injected subcutaneously (s.c.) on days 1, 3 and 7. Intraperitoneal injections of dexamethasone were administered daily until day 9 (injections were given on day 1, 2, 3, 4, 5, 6, 7, 8 and 9). The F8 antibody was in diabody format and conjugated to murine IL4. The sequence of this antibody is shown in SEQ ID NO: 21. The sequence of the L19-IL10 antibody is shown in SEQ ID NO: 63. L19-IL10 was administered subcutaneously in an amount of 200 μg, as this is the dose and route of administration which has been shown to be therapeutically effective in the literature (Trachsel et al., 2007; Schwager et al., 2009).

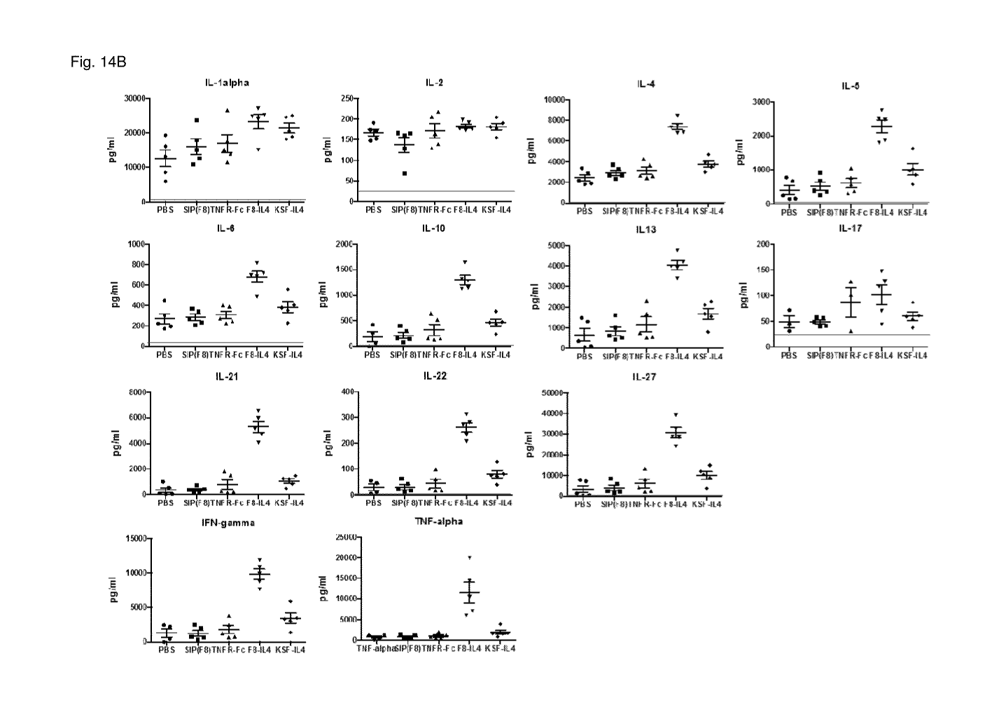

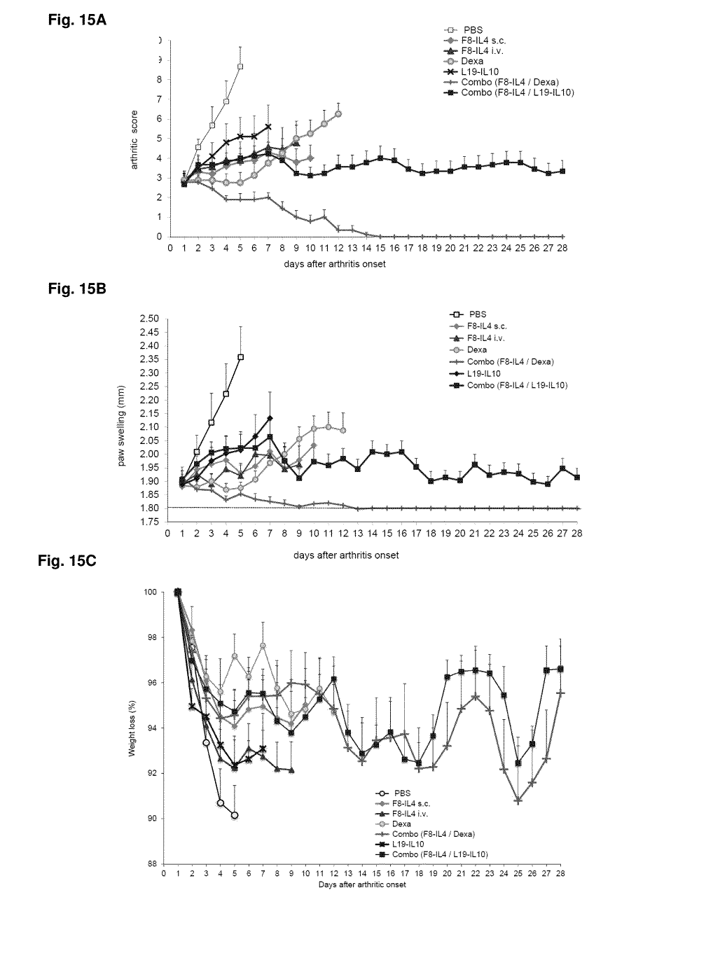

Mice were analyzed per group (n≧8) daily and the arthritic sore, the thickness of inflamed paws and weight was monitored. The results of these experiments are shown in FIG. 15. Mice were sacrificed in accordance with local regulations due to arthritic score (>6 for more than 4 days) and weight loss (>15%). This resulted in the early termination of some of the treatment schedules.

As shown in FIG. 15, treatment with a combination of F8-IL4 and dexamethasone resulted in a more potent disease-modulating effect than treatment with either F8-IL4 or dexamethasone alone. Mice treated with F8-IL4 in combination with dexamethasone lost less weight than mice treated with either F8-IL4 alone, indicating that the combination treatment was well tolerated. Mice treated with F8-IL4 in combination with dexamethasone also exhibited a significantly lower arthritic score and less severe paw swelling than mice treated with either F8-IL4 or dexamethasone alone. In contrast, mice treated with a combination of F8-IL4 and L19-IL10 only showed a moderate improvement in symptoms compared with F8-IL4 monotherapy, demonstrating that the significant further reduction in symptoms in mice treated with F8-IL4 and dexamethasone compared with mice receiving monotherapy is particularly surprising. To our knowledge this is the first time that symptoms of RA (as indicated by arthritic score and paw swelling) have been entirely suppressed (i.e. not simply reduced) in 100% of the treated mice in such an aggressive model of RA. F8-IL4 in combination with a glucocorticoid, such as dexamethasone, thus represents a very promising candidate for treatment of RA in humans.

Treatment with F8-IL4 also exhibited a more potent disease-modulating effect than treatment with L19-IL10, as indicated by the fact that mice treated with F8-IL4 exhibited less paw swelling, weight loss, and a greater reduction in arthritic score than mice treated with L19-IL10. This is particularly surprising, as twice as much L19-IL10 was administered to the mice compared with F8-IL4 (the molecular weight of these two conjugates is almost identical). The reduced weight loss in mice treated with F8-IL4 further indicates that F8-IL4 treatment was well tolerated. F8-IL4 thus also represents a promising candidate for treatment of RA in humans.

Examples Relating to the Treatment of Cancer using Antibodv-IL4 Conjugates

In the description of the below experiments, data are expressed as the mean (±SEM). Differences in tumor volume, % ID/g and Tumor-to-blood ratio between therapeutic groups were compared using Graphpad Prism's repeated measures (mixed model) ANOVA analysis.

### Example 5

### Cloning, Expression and In Vitro Characterization of KSF-IL4

A fusion protein, KSF-IL4, containing the antibody KSF (Frey et al. 2011) (specific to hen egg lysozyme and used as negative control in the experiments) in a stable non-covalent homodimeric diabody format (i.e., scFv fragment with a 5-amino acid linker between VH and VL domains), sequentially fused at the C-terminus to murine interleukin 4 (gene from Source BioScience) via a flexible 15 amino acid linker (SEQ ID NO: 24), was prepared. The gene encoding the KSF antibody and the gene encoding murine IL4 (SEQ ID NO: 51) were PCR amplified and PCR assembled using standard procedures as described for F8-IL4 above to prepare KSF-IL4 (SEQ ID NO: 44). The product was again ligated into the mammalian expression vector pcDNA3.1(+) (Invitrogen) by a HindIII/NotI restriction site.

The fusion protein was expressed and purified to homogeneity as described for F8-IL4 above. Purity of KSF-IL4 was confirmed by SDS-PAGE and size exclusion chromatography, again as described for F8-IL4 above. KSF-IL4 also retained a high-affinity for the cognate antigen, as revealed by surface plasmon analysis (BIAcore) on an EDA antigen-coated sensor chip.

### Example 6

### Biological Cytokine Activity of F8-IL4 and KSF-IL4

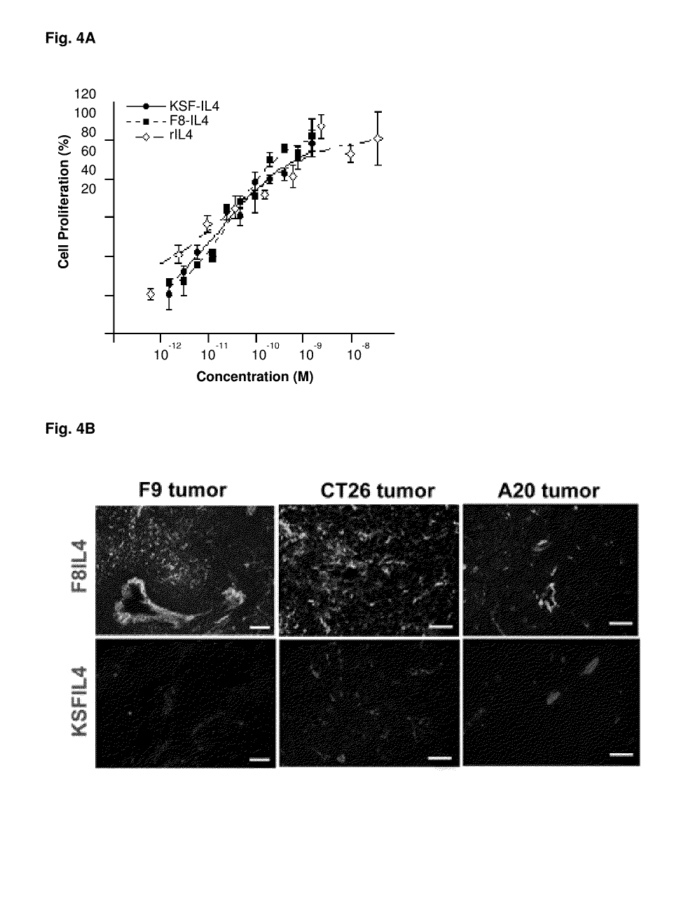

The biological activity of murine IL4 was determined by its ability to stimulate the proliferation of CTLL2 cells. CTLL2 cells were grown according to the supplier's protocol. Cells (20000 cells/well) were seeded in 96-well plates in the culture medium supplemented with varying concentrations of recombinant murine IL4 (eBioscience), F8-IL4 or KSF-IL4. After incubation at 37° C. for 24 h, cell proliferation was determined with Cell Titer Aqueous One Solution (Promega). As shown in FIG. 4A, the biological cytokine activity of F8-IL4 and KSF-IL4 was comparable to that of recombinant murine IL4 (FIG. 4A).

### Example 7

### Immunofluorescence Studies of F8-IL4 and KSF-IL4 on Tumour Sections

For this, as well as the other experiments described below, F9 teratocarcinoma (CRL-1720; ATCC, Molsheim Cedex, France), CT26 colon carcinoma (CRL-2638; ATCC, Molsheim Cedex, France) and A20 lymphoma (TIB-208; ATCC, Molsheim Cedex, France) were grown according to the supplier's protocol and tumour cells implanted subcutaneously in the flank using 25×10⁶ cells (F9), 2×10⁶ cells (CT26) or 8×10⁶ cells (A20). F9 teratocarcinoma cells were implanted into 129/SvEv mice (Charles River, Sulzfeld, Germany), CT26 colon carcinoma cells were implanted into Balb/c mice (Elevage Janvier, Le Genest-St-Isle, France) and A20 lymphoma cells were implanted into Balb/c mice (Elevage Janvier, Le Genest-St-Isle, France). Mice were sacrificed when tumour volumes reached a maximum of 2000 mm³.

For immunofluorescence, 10 μm cryostat sections of untreated tumours were fixed in ice-cold acetone and stained with biotinylated F8-IL4 respectively KSF-IL4 and rat anti-CD31 (BD Bioscience). For detection streptavidin-Alexa Fluor 488 and anti-rat-Alexa Fluor 594 (Invitrogen) were used. Slides were mounted with fluorescent mounting medium (Dako) and analyzed with an Axioskop2 mot plus microscope (Zeiss). Immunofluorescence showed that F8-IL4 (but not KSF-IL4) was able to recognize tumour neovascular structures in murine F9, CT26 and A20 tumours (FIG. 4B).

### Example 8

### Biodistribution Studies

The in vivo targeting performance was assessed by quantitative biodistribution where 15 μg of radioiodinated fusion protein was injected i.v. into the lateral tail vein of immunocompetent 11-12 week old female Sv129Ev mice (obtained from Charles River [Germany]), bearing sub-cutaneously grafted F9 tumours (for labelling protocol, see (Pasche et al. 2011)). Mice were sacrificed 24 hours after injection, organs were excised, weighed and radioactivity was measured using a Packard Cobra γ counter. Radioactivity of organs was expressed as percentage of injected dose per gram of tissue (% ID/g±SEM).

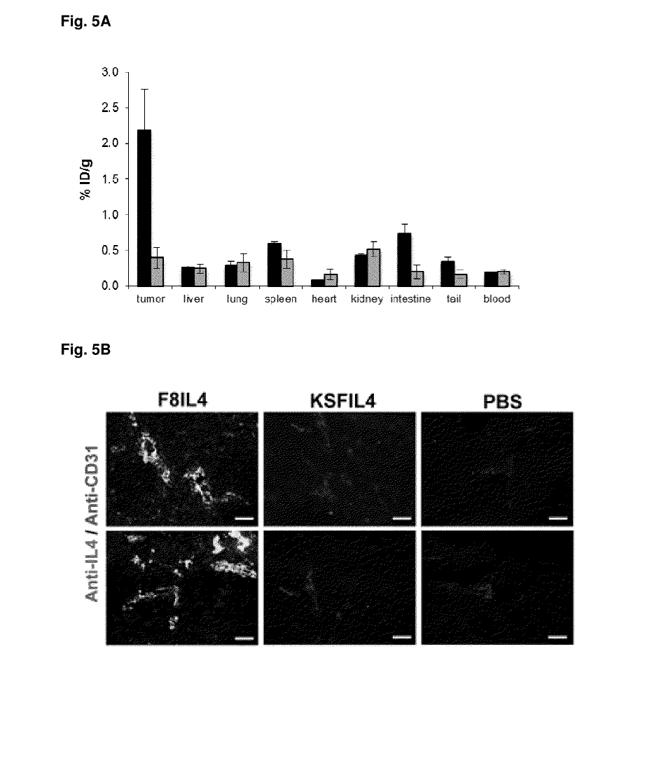

Analysis of percentage injected dose per gram of tissue 24 hours after intravenous administration showed a preferential accumulation of F8-IL4 at the tumour site, which was not observed for the negative control KSF-IL4 fusion protein (p<0.001) (FIG. 5A). An ex vivo immunofluorescence analysis of tumour sections following intravenous administration of the immunocytokines confirmed a preferential accumulation of F9-IL4 on CD31-positive tumour neo-vascular structures (FIG. 5B).

### Example 9

### Therapy Studies

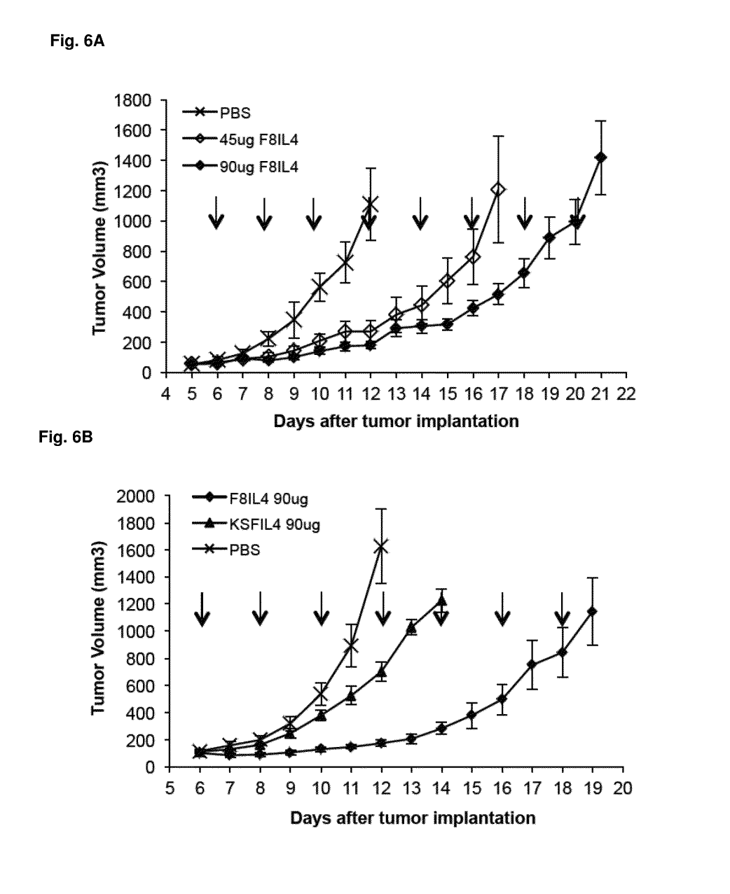

In a dose finding study, F9 tumour-bearing mice were randomly grouped (n=5) and injected intravenously (i.v.) into the lateral tail vein when tumours were clearly palpable. Specifically, the anti-tumour activity of F8-IL4 was tested by intravenous injections into the lateral tail vein every second day at doses of 45 μg and 90 μg, starting when tumours were 50 mg in weight. The therapeutic antibodies were all dissolved in phosphate buffered saline (PBS) and this buffer was also used in the negative control treatment groups. The mice were monitored daily and tumour volume was measured with a calliper (volume=length×width²×0.5). The higher dose exhibited the best tumour retardation results (FIG. 6A). A higher dose could not be tested, due to insufficient solubility of the product. A comparison between F8-IL4 and KSF-IL4 revealed that the EDA-specific immunocytokine exhibited a substantially more potent (for P values, see below) therapeutic effect (FIG. 6B).

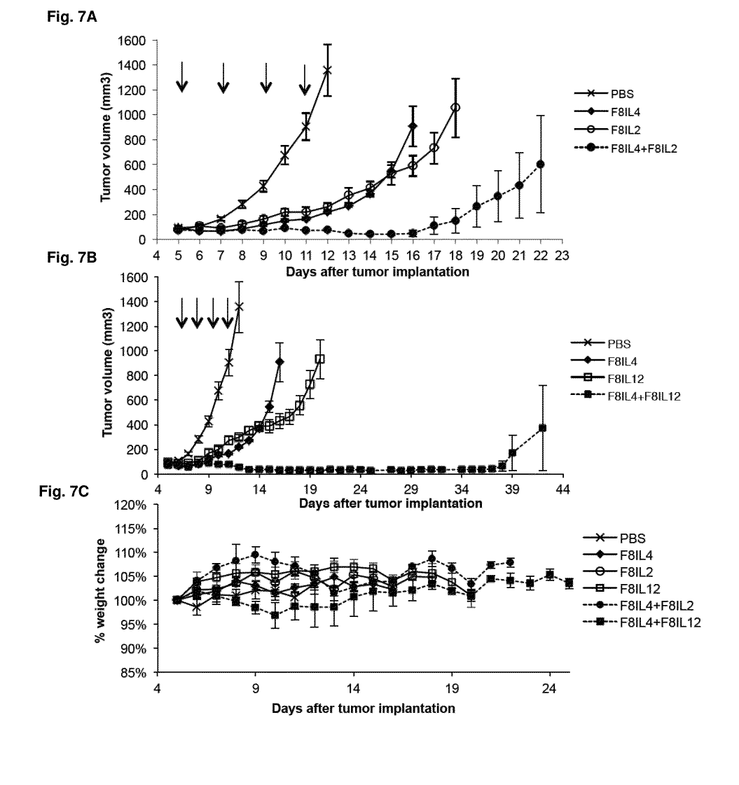

F9 tumours were not cured by F8-IL4 treatment alone. Treatment of F9 tumour-bearing mice using F8-IL4 plus F8-IL2 (Frey et al., 2010) was therefore performed. FIG. 7A shows the therapy results obtained with four injections of F8-IL4 (90 μg) and F8-IL2 (20 μg) (SEQ ID NO: 50).

Substantial tumour growth retardation was observed for the combination regimen, which led to complete tumour eradications in 2/5 mice. Upon re-challenge with F9 cells, however, both animals developed tumours. The combination of F8-IL4 with IL12-F8-F8 (8.6 μg) (SEQ ID NO: 48), a newly-developed IL12-based immunocytokine which had shown activity in several mouse models of cancer (Pasche et al., Clin. Cancer Research, 2012) was then studied (FIG. 7B). Here, the combination treatment led to complete tumour eradications in 4/5 mice. Again, in a re-challenge experiment, all animals developed new tumour lesions.

The observation that the F8-mediated targeted delivery of IL4 and IL12 may lead to additive anti-cancer effects was counter-intuitive and unexpected, since the two cytokines are thought to mediate opposite effects on the regulation of T cell activity. While interleukin 4 primes naïve T cells into a Th2 response, IL12 promotes T cell differentiation into IFNγ-producing Th1 cells, thereby activating an opposing arm of the adaptive immune response. (26). In spite of the different immunobiology of the two cytokines, both F8-IL4 and IL12-F8-F8 mediated comparable patterns of leukocyte infiltration into the tumour mass and displayed a potent single-agent therapeutic activity.

The treatment with F8-IL4, F8-IL2, IL12-F8-F8 and the corresponding combinations was well tolerated, as reflected by the fact that weight loss was <5% (FIG. 7C).

F9 Teratocarcinoma: Dose-Escalation of F8-IL4

| | | | |
|---|---|---|---|
| F8-IL4 45 μg vs PBS: | from day | 10 | p < 0.05 |
| | | 11 | p < 0.001 |
| | | 12 | p < 0.0001 |
| F8-IL4 90 μg vs PBS: | from day | 10 | p < 0.01 |
| | | 11 | p < 0.0001 |
| F8-IL4 45 μg vs F8-IL4 90 μg: | from day | 17 | p < 0.0001 |

F9 Teratocarcinoma: F8-IL4 vs KSF-IL4

| | | | |
|---|---|---|---|
| F8-IL4 vs PBS: | from day | 10 | p < 0.01 |
| | | 11 | p < 0.001 |
| | | 12 | p < 0.0001 |
| KSF-IL4 vs PBS: | from day | 11 | p < 0.001 |
| | | 12 | p < 0.0001 |
| F8-IL4 vs KSF-IL4: | from day | 10 | p < 0.0001 |

F9 Teratocarcinoma: F8-IL4, F8-IL2, F8-IL12 and Combinations

| | | | |
|---|---|---|---|
| F8-IL4 vs PBS: | from day | 9 | p < 0.0001 |
| F8-IL2 vs PBS: | from day | 9 | p < 0.0001 |
| F8-IL12 vs PBS: | from day | 9 | p < 0.0001 |
| F8-IL4/F8-IL2 vs PBS: | from day | 13 | p < 0.01 |
| | | 14 | p < 0.0001 |
| F8-IL4/F8-IL12 vs PBS: | from day | 13 | p < 0.01 |
| | | 14 | p < 0.0001 |
| F8-IL4 vs F8-IL2: | from day | 16 | p < 0.0001 |
| F8-IL4 vs FR-IL12: | from day | 16 | p < 0.0001 |
| F8-IL2 vs F8-IL12: | from day | 17 | p < 0.01 |
| | | 18 | p < 0.0001 |
| FB-IL4 vs F8-IL4/F8-IL2: | from day | 9 | p < 0.01 |
| | | 10 | p < 0.0001 |
| FR-IL2 vs F8-IL4/F8-IL2: | from day | 13 | p < 0.05 |
| | | 14 | p < 0.001 |
| | | 15 | p < 0.0001 |
| F8-IL4 vs F8-IL4/F8-IL12: | from day | 9 | p < 0.001 |
| | | 10 | p < 0.0001 |
| F8-IL2 vs F8-IL4/F8-IL12: | from day | 13 | p < 0.05 |
| | | 14 | p < 0.001 |
| | | 15 | p < 0.0001 |
| F8-IL12 vs F8-IL4/F8-IL2: | from day | 13 | p < 0.01 |
| | | 14 | p < 0.001 |
| | | 15 | p < 0.001 |
| | | 16 | p < 0.0001 |
| F8-IL12 vs F8-IL4/F8-IL12: | from day | 11 | p < 0.05 |
| | | 12 | p < 0.001 |
| | | 13 | p < 0.0001 |
| F8-IL4/F8-IL2 vs F8-IL4/F8-IL12: | from day | 20 | p < 0.05 |
| | | 21 | p < 0.001 |

### Example 10

### Immunofluoreseence Studies of Treated Tumours

For ex vivo detection of the localization of the IL4-based immunocytokines and of tumour infiltrating cells after therapy, mice were injected three times with the appropriate proteins as for therapy experiments and sacrificed two days after the last injection. Tumours were excised, embedded in cryoembedding medium (Thermo Scientific) and cryostat sections (10 μm) were stained using the antibodies: Anti-IL4 antibody (eBioscience), CD45 (leukocytes, BD Biosciences), CD4 (CD4+Tcells, BioXCell), CD8 (CD8+Tcells, BioXCell), F4/80 (macrophages, Abcam), Asialo GM1 (NK cells, Wako Pure Chemical Industries), CD45R (B cells, eBioscience), Foxp3 (eBioscience), CD31 (Santa Cruz); and detected with AlexaFluor488 respectively AlexaFluor594 coupled secondary antibodies (Invitrogen). Slides were mounted with fluorescent mounting medium (Dako) and analyzed with an Axioskop2 mot plus microscope (Zeiss).

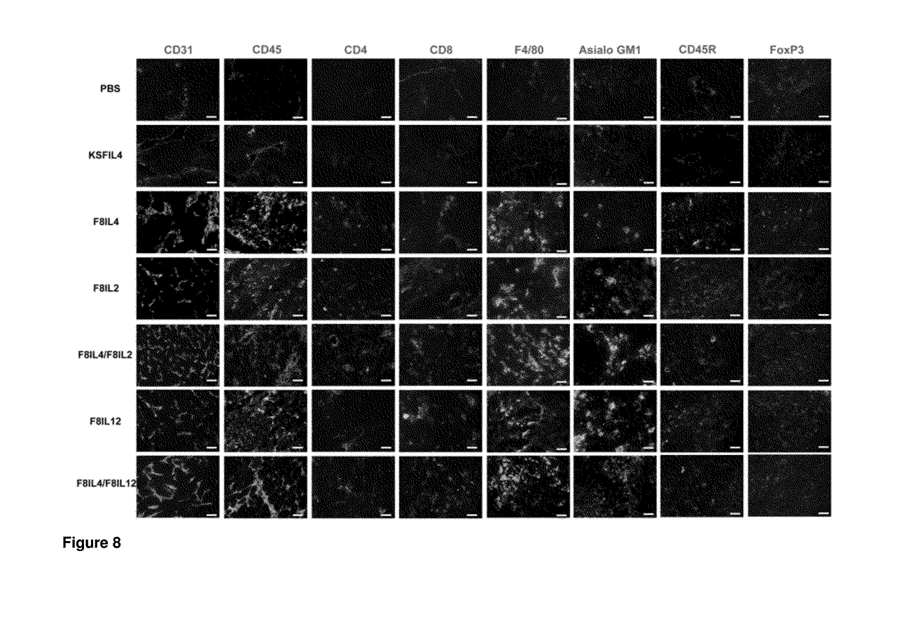

A microscopic analysis of tumour sections following immunocytokine treatment, staining for vascular structures (CD31), CD45-positive leukocytes, CD4 and CD8-positive lymphocytes, F4/80-positive cells (mainly macrophage), Asialo GM1 positive cells (mainly NK cells), CD45R-positive cells (mainly B cells) and FoxP3 (a marker expressed in regulatory T cells), revealed a rich infiltrate of a variety of leukocyte in the immunocytokine treatment groups, without substantial changes in vascular structures or in FoxP3-positive cells (FIG. 8).

### Example 11

### In Vivo Studies in CT26 Tumour-Bearing Mice

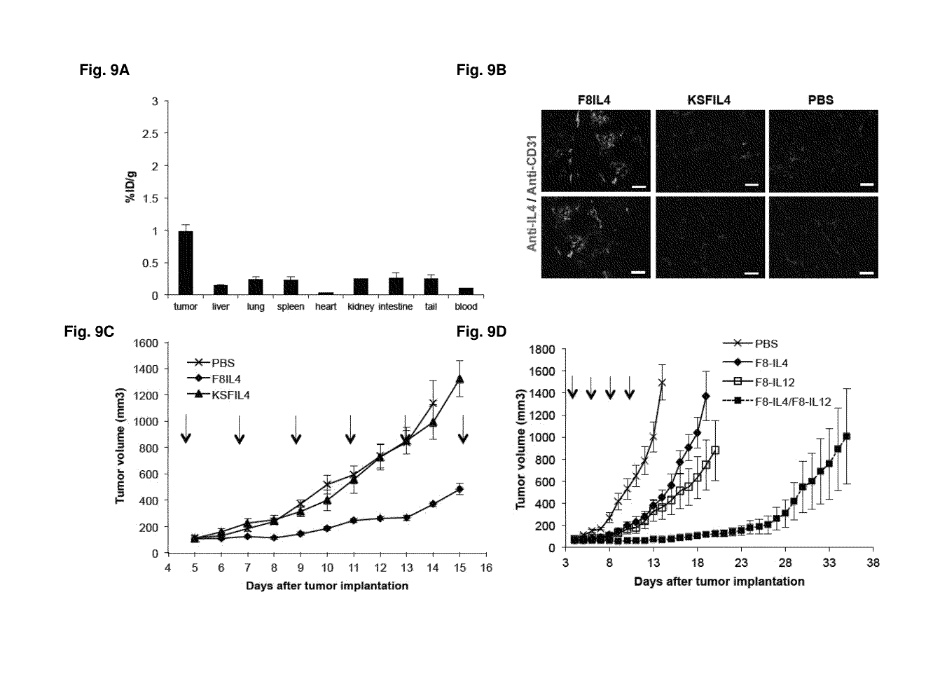

In order to study a second immunocompetent mouse model of cancer, the inventors focused on Balb/c mice (obtained from Elevage Janvier [France]) bearing subcutaneous CT26 tumours of colorectal origin. As for F9 tumours, a preferential tumour targeting was observed for F8-IL4, although with lower % ID/g in neoplastic lesions (FIG. 9A). A microscopic ex vivo immunofluorescence analysis confirmed that only F8-IL4 had preferentially accumulated at the tumour site (FIG. 9B). Also in this model, F8-IL4 displayed a superior therapeutic performance compared to KSF-IL4 (for P values, see below), which was not better than saline (FIG. 9C). Substantial tumour growth retardation could be observed in the F8-IL4 plus IL12-F8-F8 combination group, with complete cures in 2/5 mice (FIG. 9D), without any detectable weight loss. A microscopic analysis of tumour sections following immunocytokine treatment confirmed a rich leukocyte infiltrate, in analogy to the data presented for F9 tumours.

CT26 Colon Carcinoma: F8-IL4 vs KSF-IL4

| | | | |
|---|---|---|---|
| F8-IL4 vs PBS: | from day | 10 | p < 0.01 |
| | | 11 | p < 0.01 |
| | | 12 | p < 0.0001 |

| | |
|---|---|
| KSF-IL4 vs PBS: | not statistically significant |

| | | | |
|---|---|---|---|
| F8-IL4 vs KSF-IL4: | from day | 11 | p < 0.01 |
| | | 12 | p < 0.0001 |

CT26 Colon Carcinoma: F8-IL4 vs F8-IL12

| | | | |
|---|---|---|---|
| F8-IL4 vs PBS: | from day | 9 | p < 0.05 |
| | | 10 | p < 0.001 |
| | | 11 | p < 0.0001 |
| F8-IL12 vs PBS: | from day | 10 | p < 0.01 |
| | | 11 | p < 0.0001 |
| F8-IL4/F8-IL12 vs PBS: | from day | 9 | p < 0.001 |
| | | 10 | p < 0.0001 |
| F8-IL4 vs F8-IL12: | from day | 19 | p < 0.01 |
| F8-IL4 vs F8-IL4/F8-IL12: | from day | 14 | p < 0.01 |
| | | 15 | p < 0.0001 |
| F8-IL12 vs F8-IL4/F8-IL12: | from day | 13 | p < 0.01 |
| | | 14 | p < 0.01 |
| | | 15 | p < 0.0001 |

### Example 12

### In Vivo Studies in A20 Tumour-Bearing Mice

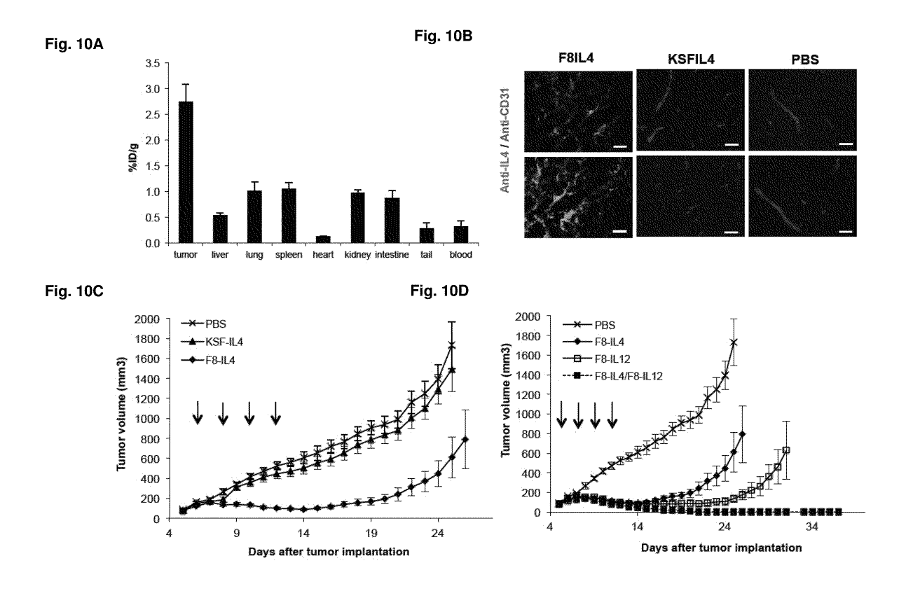

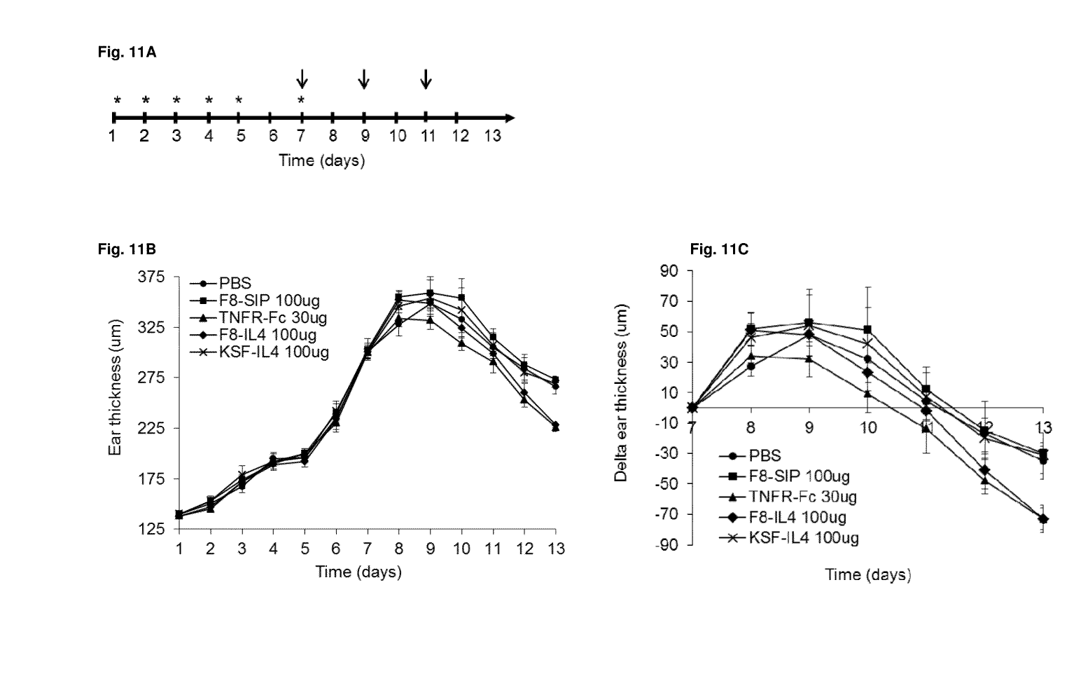

A20 murine lymphomas were studied as a third model of cancer, since not only solid tumours but also the majority of lymphomas have previously been reported to strongly express oncofetal fibronectin around vascular structures (Schliemann et al., Leuk. Res., 2009; Sauer et al. 2009; Schliemann et al., Blood, 2009). As for the previous two models, a quantitative biodistribution study and an ex vivo immunofluorescence analysis following intravenous administration confirmed a preferential accumulation of F8-IL4 around tumour neo-vascular structures (FIGS. 10A and B). In keeping with its selective tumour targeting properties, F8-IL4 displayed a superior tumour growth retardation compared to KSF-IL4 (FIG. 10C). The combination of F8-IL4 and IL12-F8-F8 led to complete eradications of implanted tumours in all treated mice (FIG. 10D), without any weight loss. However, 25 days after the beginning of therapy, small tumour nodules appeared at inguinal lymph nodes in 4/5 mice, which continued to grow. A microscopic analysis of tumour sections following immunocytokine treatment confirmed, also in this case, a rich leukocyte infiltrate. The analysis of B cell infiltration did not yield informative results with the CD45R marker, because of the B-cell lymphoma origin of A20 tumours.

A20 Lymphoma: F8-IL4 vs KSF-IL4

| | | | |
|---|---|---|---|
| F8-IL4 vs PBS: | from day | 11 | p < 0.05 |
| | | 12 | p < 0.001 |
| | | 13 | p < 0.0001 |

| | |
|---|---|
| KSF-IL4 vs PBS: | not statistically significant |

| | | | |
|---|---|---|---|
| F8-IL4 vs KSF-IL4: | from day | 12 | p < 0.05 |
| | | 13 | p < 0.01 |
| | | 14 | p < 0.001 |
| | | 15 | p < 0.0001 |

A20 Lymphoma: F8-IL4 vs F8-IL12

| | | | |
|---|---|---|---|
| F8-IL4 vs PBS: | from day | 11 | p < 0.05 |
| | | 12 | p < 0.001 |
| | | 13 | p < 0.0001 |
| F8-IL12 vs PBS: | from day | 11 | p < 0.01 |
| | | 12 | p < 0.001 |
| | | 13 | p < 0.0001 |
| F8-IL4/F8-IL12 vs PBS: | from day | 11 | p < 0.01 |
| | | 12 | p < 0.0001 |
| F8-IL4 vs F8-IL12: | from day | 22 | p < 0.05 |
| | | 23 | p < 0.001 |
| | | 24 | p < 0.0001 |
| F8-IL4 vs F8-IL4/F8-IL12: | from day | 21 | p < 0.05 |
| | | 22 | p < 0.0001 |
| F8-IL12 vs F8-IL4/F8-IL12: | from day | 28 | p < 0.05 |
| | | 29 | p < 0.001 |
| | | 30 | p < 0.0001 |

### Example 13

### In Vivo Studies in Fibrosarcoma Tumour-Bearing Mice

Fibrosarcoma Wehi-164 exponentially growing cells were harvested, repeatedly washed and re-suspended in serum-free medium prior to injection. The tumor cells were injected subcutaneously (5×10⁶) in the right flank of Balb/c mice. Tumor weights (mg=mm³) were calculated as follows: (length [mm]×width² [mm²])/2.

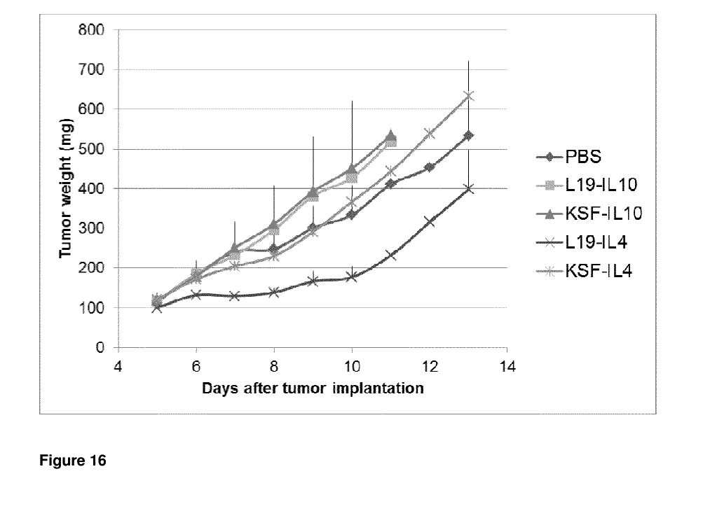

Treatment started when tumors reached approximately 100 mg. Mice received 4 cycles of therapy, every 48 h starting from day 5 after tumor cell injection. L19-IL4 (SEQ ID NO:64) was administered i.v. at the dose of 130 μg/mouse; equimolar amounts of KSF-IL4 (130 μg/mouse) (SEQ ID NO:44), KSF-IL10 (150 μg/mouse) (SEQ ID NO:65) and L19-IL10 (150 μg/mouse) (SEQ ID NO:66) were administered. Control mice received sterile vehicle solution (PBS). The results are shown in FIG. 16 (mean tumour weight over time). The number of mice in each treatment group was 3-4.

FIG. 16 shows that L19-IL4, as well as F8-IL4, can be used to treat cancer, as demonstrated by the reduced tumour weight in mice treated with this conjugate compared with the PBS control and the control KSF-IL4 antibody. The F8 and L19 antibodies both bind an extra-cellular matrix component associated with neoplastic growth and/or angiogenesis. Thus, these results show that conjugates comprising IL4 and a specific binding member which binds an extra-cellular matrix component associated with neoplastic growth and/or angiogenesis can be used in the treatment of cancer. In addition, these results show that that while untargeted IL10 and untargeted IL4 do not promote any significant therapeutic activity, targeted IL4, but not targeted IL10, shows therapeutic activity, given the reduced tumour weight in mice treated with L19-IL4 compared with those treated with L19-IL10, KSF-IL10 and KSF-IL4.

Examples Relating to the Treatment of Psoriasis using Antibody-IL4 Conjugates

### Example 14

### IMQ-Induced Inflammation Model of Psoriasis

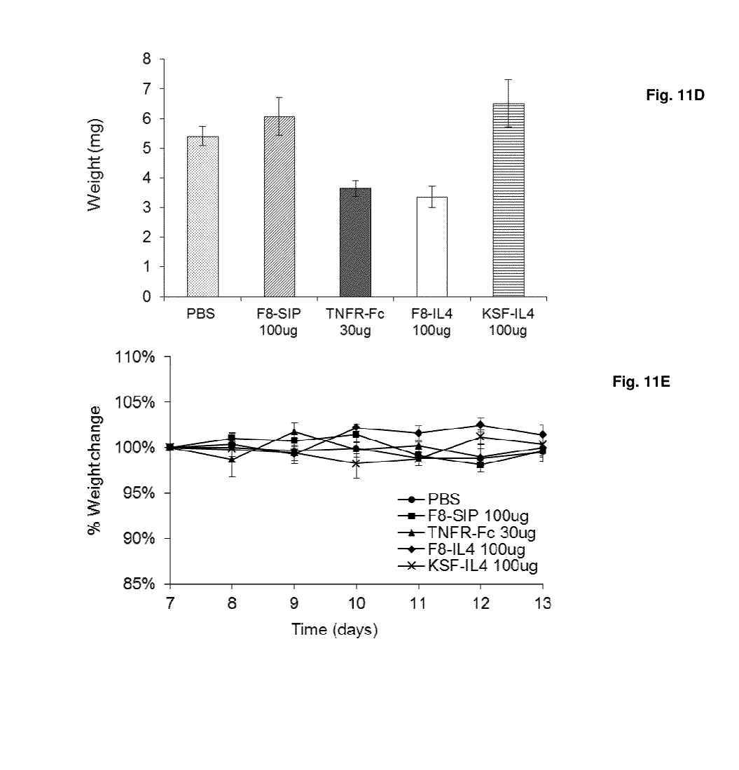

In order to evaluate the therapeutic potential of F8-IL4 in psoriasis, an imiquimod (IMQ)-induced inflammation experiment was carried out. C57BL/6 mice (Charles River, Germany) were treated on each side of each ear with 5 mg Aldara cream (containing 0.25 mg Imiquimod). The treatment schedule is illustrated in FIG. 11A. The Aldara cream was applied every day for 5 days (on day 1, 2, 3, 4 5 and 7: each application is illustrated by an asterisk in FIG. 11A). Ear thickness was measured with a caliper before the application of the cream. On day 7, mice were randomly grouped and intravenously injected with either PBS, 100 μg SIP (F8), 30 μg murine TNFR-Fc (positive control), 100 μg F8-IL4 or 100 μg KSF-IL4. Treatment was repeated on day 9 and 11 (each treatment event is illustrated by an arrow in FIG. 11A).

Ear thickness as measured over the course of the experiment is shown in FIG. 11B. Treatment with F8-IL4 significantly reduced ear thickness by day 13 compared to either treatment with PBS or KSF-IL4 (PBS vs F8-IL4=p<0.05; KSF-IL4 vs F8-IL4=p<0.05, both at day 13). Results in FIG. 11B are expressed as ear thickness in μm±SEM.

The change in ear thickness from the day of treatment initiation (day 7) is shown in FIG. 11C. Results are expressed as delta ear thickness in μm±SEM. The decrease in ear thickness of mice treated with F8-IL4 was significantly more than in mice receiving either PBS, F8-SIP or KSF-IL4 treatment.

Mice were sacrificed on day 13 and the ear draining lymph nodes were excised and weighted (FIG. 11D). Results are expressed in weight±SEM. The lymph nodes of mice treated with F8-IL4 or the positive control murine TNFR-Fc were smaller, indicating that the inflammation of the ear is not as severe as in the groups receiving F8-SIP, KSF-IL4 or PBS treatment. Statistical analysis was carried out: PBS vs F8-IL4 p≦0.05; KSF-IL4 vs F8-IL4 p≦0.001 (both at day 13).

FIG. 11 E shows the weight change of the mice during treatment. No loss of weight could be observed, what indicates that the treatment was well tolerated with no indication of toxicity. Results are expressed in weight±SEM.

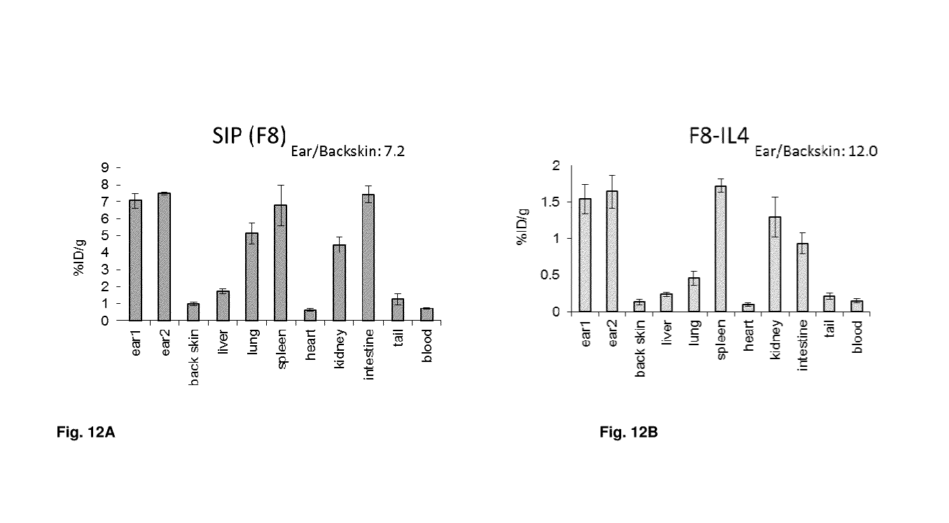

Biodistribution experiments were carried out in order to analyze the distribution of SIP (F8) and F8-IL4 in mice with IMQ-induced inflammation in the ears. On day 7 of inflammation mice were injected intravenously with 10 μg radioiodinated protein (I-125). After 24 h, mice were sacrificed and organs were excised, weighted and counted in a Packard cobra y-counter. The results are illustrated in FIG. 12A and FIG. 12B (results are expressed as % injected dose per gram). The ear to backskin ratio shows a preferential accumulation of the antibody in inflamed tissue.

All animal experiments were performed under a project license granted by the Veterinaeramt des Kantons Zürich, Switzerland (117/2011).

### Example 15

### Contact Hypersensitivity-Induced Ear Inflammation in Hemizygous K14-VEGF-A Mice

In order to further evaluate the therapeutic potential of F8-IL4 in the treatment of psoriasis, a CHS-induced ear inflammation study was carried out in K14-VEGF-A mice.

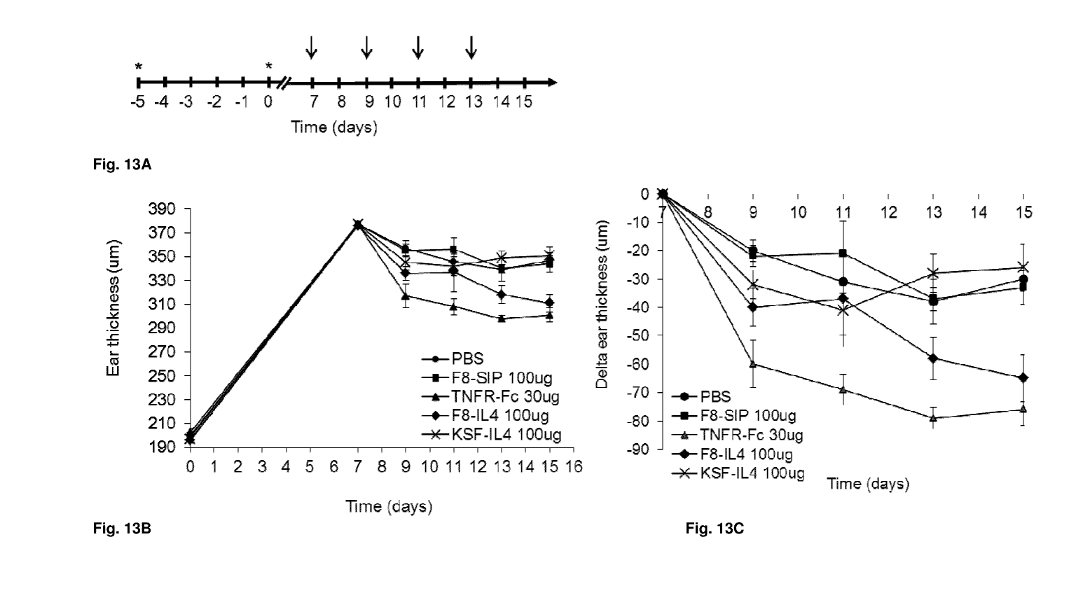

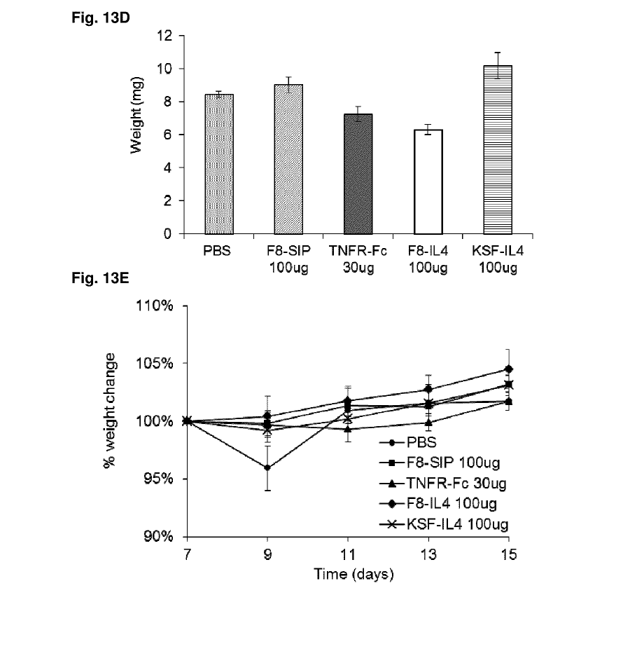

A delayed-type hypersensitivity reaction was induced in the ear skin of female FVB mice that overexpress VEGF-A in the epidermis under control of the human keratin 14 promoter as previously described (Detmar et al., 1998; Kunstfeld et al., 2004; Xia et al., 2003). Heterozygous VEGF-A transgenic mice with no pre-existing inflammatory lesions were sensitized by topical application of a 2% oxazolone (4-ethoxymethylene-2-phenyl-2-oxazoline-5-one; Sigma) solution in acetone/olive oil (4:1 vol/vol) to the shaved abdomen (50 μl) and each paw (5 μl). Five days after sensitization, the ears were challenged by topical application of 20 μl of a 1% oxazolone solution. Therapy was started on day 7 and repeated at day 9, 11 and 13. Mice were grouped and injected intravenously with PBS, 100 μg SIP (F8), 30 μg murine TNFR-Fc, 100 μg F8-IL4 and 100 μg KSF-IL4. Ear thickness was measured every other day and at day 15, mice were sacrificed and ear draining lymph nodes were weighted. The timeline for this experiment is illustrated in FIG. 13A. Each treatment event is indicated by an arrow.

Ear thickness as measured over the course of this experiment is shown in FIG. 13B. Treatment with F8-IL4 significantly reduced ear thickness by day 13 compared to either treatment with PBS or KSF-IL4 (PBS vs F8-IL4 p<0.01; KSF-IL4 vs F8-IL4 p<0.01, both at day 15). Results are expressed as ear thickness in μm±SEM.

The change in ear thickness from the day of treatment initiation (day 7) is shown in FIG. 13C. Results are expressed as delta ear thickness in μm±SEM. Treatment with F8-IL4 decreased ear thickness in mice significantly more than treatment with either FSF-IL4, F8-SIP or PBS. Statistical analysis at day 15: PBS vs F8-IL4 p<0.05; KSF-IL4 vs F8-IL4 p<0.05.

FIG. 13E shows the weight change of the mice during treatment. No loss of weight could be observed, what indicates that the treatment was well tolerated with no apparent toxicity. Results are expressed in weight±SEM.

Mice were sacrificed on day 15 and the ear draining lymph nodes were excised and weighed (FIG. 13D). Results are expressed in weight±SEM. The lymph nodes of mice treated with F8-IL4 or the positive control murine TNFR-Fc were smaller, indicating that the inflammation of the ear is not as severe as in the groups treated with F8-SIP, KSF-IL4 or PBS. Statistical analysis was carried out on results from day 15: PBS vs F8-IL4 p≦0.05, KSF-IL4 vs F8-IL4 p≦0.0001.

### Example 16

### Analysis of Cytokine Levels in Tissue Extracts from Examples 12 and 13

The levels of 13 different cytokines were measured in tissue obtained from the mouse models of psoriasis in Examples 12 and 13 in order to analyze the change in cytokine levels following treatment.

Ear tissue was obtained at the end of therapy from each mouse and processed to a tissue extract as previously described (Schwager et al., 2013). The end of therapy was day 13 for the IMQ model described in Example 14 and day 15 for the CHS model described in Example 15. Briefly, ears were cut into small pieces and suspended in a 50 mM Tris, 150 mM NaCl buffer containing complete protease inhibitor cocktail (Roche Diagnostics, Rotkreuz, Switzerland). For homogenization, a 5 mm stainless steel bead (Qiagen, Hombrechtikon, Switzerland) was added, and the tissue was homogenized in a QIAGEN Tissue Lyzer (4× 1 minute, 4° C., 30 Hz). After centrifugation (5 min, 4° C., 16,000 g), supernatant was harvested after centrifugation (5 min, 4° C., 16,000 g). Protein concentrations were determined by BCA assay (Thermo Fisher Scientific, IL) and total protein concentration of all samples was normalized. To quantify cytokine levels of treated and control mice, a multiplex bead-based flow cytometry analysis was performed using the mouse Th1/Th2/Th17/Th22 13plex FlowCytomix Multiplex kit (eBioscience). FACS analysis was performed on a BD FACS Canto (BD Bioscience, Allschwil, Switzerland) and data evaluated with FlowCytomix Pro 3.0 software (eBioscience). Using standard curves generated by the FlowCytomix Pro 3.0 software with control samples, a level of quantification was assigned for each cytokine.

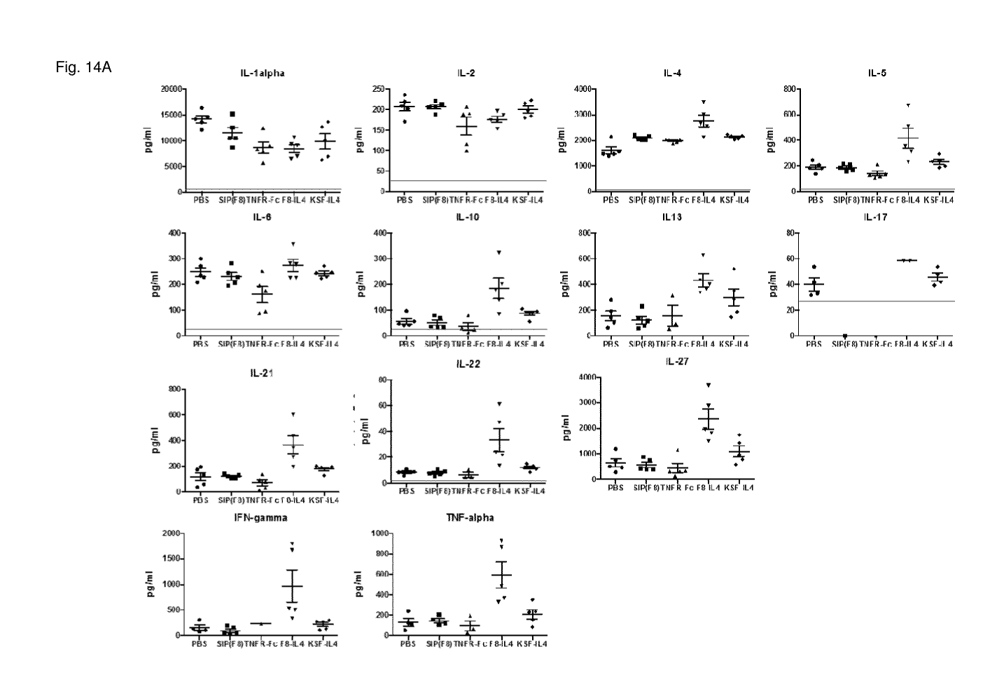

The results of the cytokine analysis are shown in FIG. 14A for the IMQ-induced inflammation model and FIG. 14B for the CHS-induced ear inflammation model. Results are expressed as the mean±SEM. The level of quantification is indicated by the narrow horizontal line across each graph.

The results in FIG. 14A and FIG. 14B shows that some cytokines, including IL10, IL13 and IL27, are up-regulated by F8-IL4 while other cytokines do not appear to show a change in concentration (e.g. IL2). Some cytokines may be down-regulated by F8-IL4, for example IL-1 alpha in the IMQ-induced psoriasis model.

Example Relating to the Treatment of Endometriosis using Antibodv-IL4 Conjugates

### Example 17

### Treatment of Endometriosis

Endometriosis was induced in sixty eight-week old female Balb/c mice according to the protocol described by Somigliana et al. (1999).

One day after the implantation of endometrial tissue, pairs of mice (who received tissue from the same donor) were treated either with intravenous (i.v.) injections of F8-murine IL4 (200 μg/mouse; SEQ ID NO: 21) or PBS (group 1); or with i.v. injections of KSF-murine IL4 (200 μg/mouse; SEQ ID NO: 44) or PBS (group 2). The same treatment was repeated at days 4 and 7. Mice were sacrificed at day 15. The total number of lesions, as well as the size of the single lesions, in the mice treated with F8-IL4 or KSF-IL4 was compared with number and dimension of the lesions in the mice treated with PBS, in accordance with Somigliana et al. (1999). Specifically, the lesions in each mouse treated with F8-IL4 or KSF-IL4 were compared with those in the mouse belonging to the same pair which had received PBS treatment.

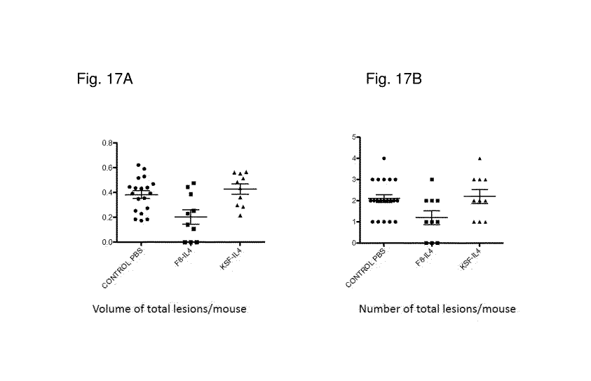

As shown in FIG. 17, F8-IL4 treatment resulted in a statistically significant reduction in both the volume [measured in cm³] (FIG. 17A) and the number (FIG. 17B) of the endometriotic lesions in the mice compared with the PBS-treated control mice. In three mice out of ten, the administration of F8-IL4 completely cured the disease. In contrast, treatment of mice with KSF-IL4 did not have a significant effect on the volume [cm³] or number of endometriotic lesions compared with the mice treated with PBS.

Example Relating to the Treatment of Multiple Sclerosis using Antibodv-IL4 Conjugates

### Example 18

### Treatment of Multiple Sclerosis

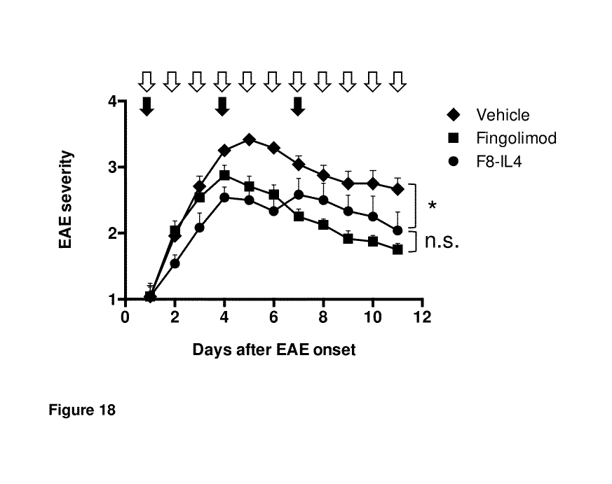

36 C57/BL6 mice were immunized with MOG₃₅₋₅₅/CFA and pertussis toxin by injection as reported in McCarthy et al. (2012) to induce experimental autoimmune encephalomyelitis (EAE), a model for multiple sclerosis (MS). Mice were scored daily and using a grading system from 0 to 5 reflecting progressive paralysis (McCarthy et al., 2012). Any mouse with newly developed clinical signs of EAE was assigned to one of three groups in a balanced manner. Treatment started on the day of assignment to a group (first day after EAE onset). Mice in the F8-murine IL4 treatment group (SEQ ID NO: 21) (indicated by circles in FIG. 18) received a total of three i.v. injection of 200 μg each (200 μl of a 1 mg/ml solution), every third day (see black arrows in FIG. 18). Mice in the Fingolimod (FTY720) treatment group (indicated by squares in FIG. 18) received daily oral gavage at a dose of 1 mg/kg (see open arrows in FIG. 18). Mice in the vehicle (PBS) group (indicated by diamonds in FIG. 18) received 200 μl of PBS i.v. in accordance with the F8-IL4 treatment schedule (see black arrows in FIG. 18).

FIG. 18 shows that F8-IL4 treatment significantly reduced EAE severity compared with mice treated using PBS. EAE severity in the mice treated with F8-IL4 was also not significantly different from that in mice treated with fingolimod (as determined by a two-way analysis of variance [ANOVA] followed by Bonferroni correction; in FIG. 18, *=P<0.05, n.s.=not significant). Clinical trial and post-marketing data suggest that fingolimod is a safe, effective, and well-tolerated treatment for MS. Regulatory approval for fingolimod as a disease-modifying therapy for relapsing forms of MS was granted in 2010. It was approved as first-line therapy without restriction in the United States and Switzerland (Mary A. Willis and Jeffrey A. Cohen. Semin. Neurol., 2013). F8-IL4 thus shows the same therapeutic activity as fingolimod, the gold standard for the treatment of MS, even though F8-IL4 was administered only every third day (3 times in total), compared with the daily administration of fingolimod over the 11 days of the study (11 administrations in total).

Example Relating to the Treatment of Diabetes using Antibody-IL4 Conjugates

### Example 19

### Treatment of Diabetes

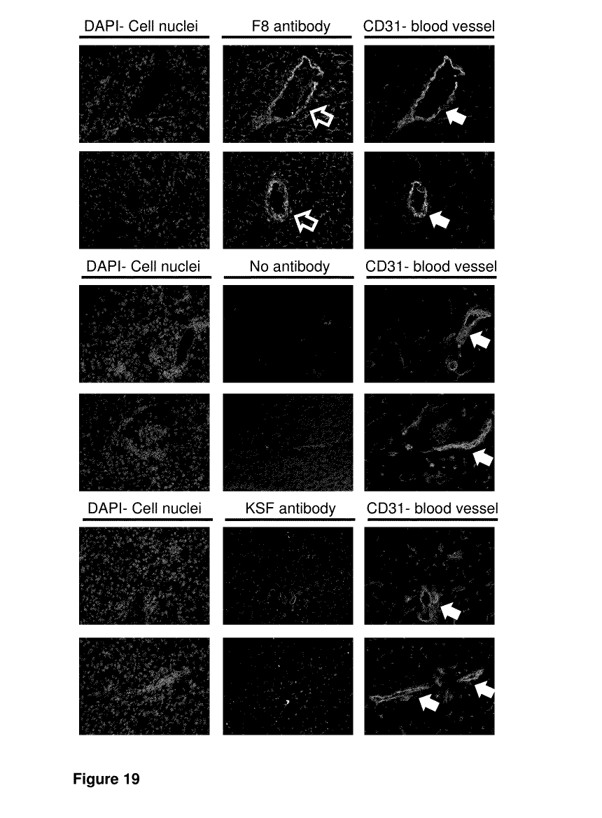

Type 1 diabetes is an autoimmune disease characterized by progressive destruction of pancreatic beta-cells. IL4 has been previously proposed as a potential medicament for preventing diabetes mellitus type 1 (Walz et al. 2002). Six week old C57BL/6J male mice were treated for 5 consecutive days with Streptozotocin (50 mg/kg by intraperitoneal [i.p.] injection). Blood glucose levels were recorded daily and mice were deemed diabetic if their non-fasted blood glucose levels were >300-400 mg/decilitre (dL). Pancreatic tissue was collected from the diabetic mice and snap frozen before cutting and staining. The staining procedure was performed according to the method set out in Pfaffen et al., Eur. J. Nucl. Med. Mol. Imaging (2010). Briefly, purified antibodies in SIP format (2 μg/ml), including antibody F8 (SEQ ID NO: 69), were added onto the sections and detected with a rabbit anti-human-IgE antibody (Dako). A further incubation with the fluorescently labelled goat anti-rabbit IgG Alexa Fluor 488 antibody (Life Technologies) was performed prior to imaging. Blood vessels and cell nuclei were detected using a rat anti-mouse CD31 antibody (BD Bioscience) followed incubation with a donkey anti-rat Alexa 594 antibody (Life Technologies), and DAPI, respectively. The filled white arrows in FIG. 19 indicate CD31⁺ blood vessels. EDA⁺ positive staining of vascular structures is indicated by the white open arrows in FIG. 19. FIG. 19 shows colocalization of EDA and CD31 in the pancreas of diabetic mice. This colocalization demonstrates that IL4 can be targeted to the perivascular space in the pancreas of diabetic patients by means of the F8 antibody. This was surprising, as it was not know that the EDA isoform of fibronectin, to which the F8 antibody binds, is expressed in the diabetic pancreas.

Example Relating to the Preparation of Unglycosylated Antibody-IL4 Conjugates

### Example 20

### Preparation, Characterization and Biodistribution of an Unglycosylated F8-hIL4 Mutant

Recombinant proteins for clinical applications are mainly produced using mammalian cell culture systems because of the capacity of eukaryotic cells for proper protein folding, assembly and post translational modifications. However, due to the ability of mammalian cells to glycosylate proteins at specific residues, manufacturing methods based on mammalian cells can result in highly heterogeneous recombinant proteins that differ in the carbohydrate components which are attached to them.

Due to regulatory concerns, tight control over the production process is required to achieve batch to batch consistency in the glycoform profile of recombinant proteins. In addition, detailed analysis of the protein glycoforms is needed, resulting in extended developmental timelines and increased production costs.

An unglycosylated variant of F8-human IL4 (F8-hIL4) would therefore offer important advantages with regard to production and characterization of the final therapeutic product. Unglycosylated proteins are produced as a single molecular species, thereby avoiding the need for controlling the glycoform profile of the protein product, and strongly simplifying the production process. In addition, highly homogeneous preparations of protein therapeutics are desirable, as they may have more predictable pharmacokinetics/pharmacodynamics (PK/PD) and potentially improved in vivo efficacy and targeting.

Because theoretical predictions indicated that human IL4 may be glycosylated at position 38, a mass spectrometry study was performed to investigate whether F8-hIL4 is glycosylated. In addition, a mutant in which the asparagine (N) at position 284 of F8-hIL4 (corresponding to position 38 of hIL4) was substituted with a glutamine (Q) was prepared (SEQ ID NO: 68).

Wild type F8-hIL4 (SEQ ID NO: 22) and the F8-hIL4 N284Q mutant (SEQ ID NO: 68) were analyzed as intact proteins by electrospray ionization (ESI) mass spectrometry with a Q Exactive mass spectrometer. In brief, sample buffer of protein solutions was exchanged against ultrapure water with VivaSpin 6 columns (GE Healthcare) with a cut-off of 30 kDa. Buffer exchange was performed on 200 μg of a protein solution. After diluting the buffer approximately 1000-fold, the concentration of the protein solution was checked by UV absorption and diluted to a final concentration of 0.015 mg/ml in 50% acetonitrile (CAN), 0.2% FA. This solution was introduced into a Q Exactive mass spectrometer (Thermo Scientific) by direct infusion with a flow rate of 5-10 μl/min. Spectra were recorded with the tune software (Thermo Scientific) of the mass spectrometer. Typically 100-1000 spectra were integrated and deconvoluted according to the manufacturer's instructions with the Protein Deconvolution 2.0 software (Thermo Scientific).

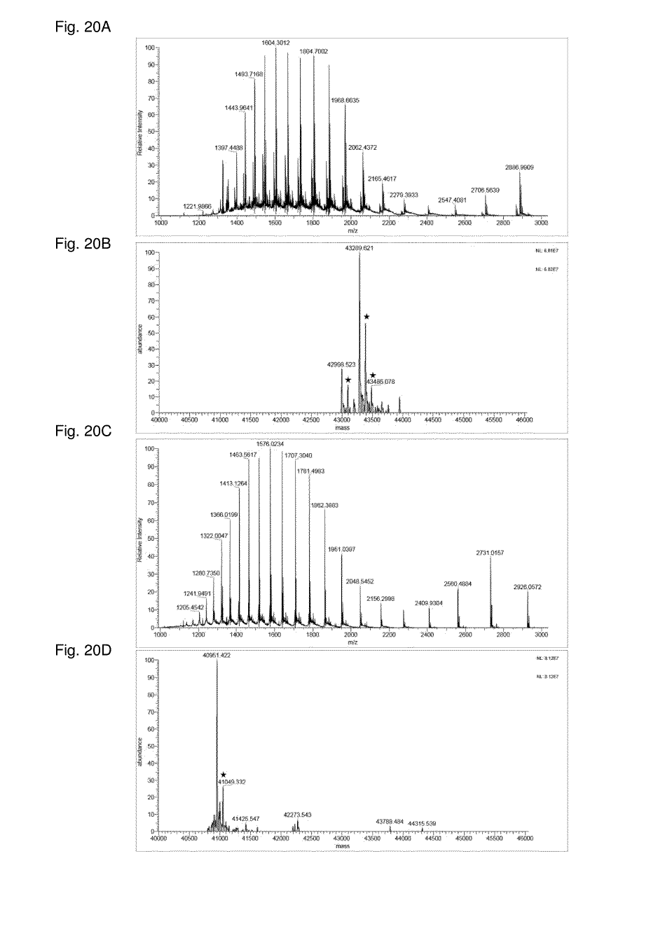

FIG. 20 shows the integrated mass spectra for wild-type F8-hIL4 (A) and the F8-hIL4 N284Q mutant (C). Deconvoluted spectra for wild-type F8-hIL4 (B) and the F8-hIL4 N284Q mutant (D) are also shown. Wild-type F8-hIL4 presents two major species at 43289.6 and 42998.5 Da in the deconvoluted spectrum. These masses are significantly higher than the expected mass of wild-type F8-hIL4 with five disulfide bonds (40938.0 Da), indicating the presence of N-linked glycosylations. The deconvoluted spectrum of the F8-hIL4 N284Q mutant, however, identifies only one single species with a mass of 40951.4 Da, which corresponds with the theoretical mass of the F8-hIL4 N284Q mutant with five disulfide bonds (40952.0 Da). Common ESI adduct peaks are indicated with an asterisk (*). FIG. 20D thus clearly shows that, unlike wild-type F8-hIL4, the F8-hIL4 N284Q mutant is not glycosylated.

After determining that the F8-hIL4 N284Q mutant is not glycosylated, the inventors tested whether this mutant retains comparable targeting properties to wild-type F8-hIL4.

F9 teratocarcinoma cells (available from ATCC under accession number CRL-1720) were grown according to the supplier's protocol. 11-12 weeks old female 129/SvEv mice were obtained from Charles River (Sulzfeld, Germany). Tumor cells were implanted subcutaneously in the flank of the mice using 25×10⁶ cells.

The in vivo targeting performance of F8-hIL4, and its unglycosylated variant F8-hIL4-N284Q were evaluated in quantitative biodistribution studies. The antibodies were radioiodinated using ¹²⁶I and Chloramine T hydrate according to the protocol of Pasche et al. 2011. 10 μg of each radioiodiated antibody were injected into the lateral tail vein of the mice. Injected mice were sacrificed 24 hours after injection. Organs were then excised and radioactivity was counted in Cobra gamma counter (Packard, Meriden, Conn.). Radioactivity was expressed as a percentage of the injected dose per gram of tissue (% ID/g±SE).

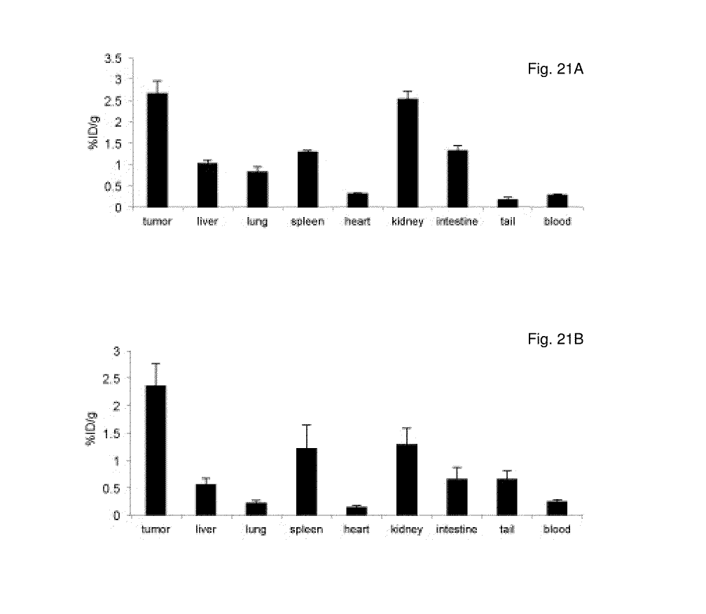

FIG. 21 shows a comparable preferential accumulation at the tumor site for F8-hIL4 (A) and its corresponding unglycosylated variant F8-hIL4-N284Q (B), indicating that the two conjugates have comparable targeting properties in vivo.

Conclusions

The observation of a potent and well-tolerated therapeutic activity of F8-IL4 in mouse models of RA, cancer and psoriasis provides a rationale for the use of the corresponding fully human immunocytokine in patients with RA, psoriasis, solid tumours and lymphomas, as well as polymorbid patients. These clinical development activities are supported by the fact that the F8 antibody recognizes the human and murine cognate antigen with identical affinity, and by the observation that the EDA domain of fibronectin is strongly expressed in a variety of different malignancies, as well as RA patients (Pedretti et al., 2009; Schliemann et al., Leuk. Res., 2009; Rybak et al., 2007; Pedretti et al., 2010; Schwager et al., 2011).

Sequence listing

Nucleotide sequence of F8 CDR's
F8 CDR1 VH-
(SEQ ID NO: 1)
CTGTTTACG
F8 CDR2 VH-
(SEQ ID NO: 2)
AGTGGTAGTGGTGGTAGC
F8 CDR3 VH-
(SEQ ID NO: 3)
AGTACTCATTTGTATCTT
F8 CDR1 VL-
(SEQ ID NO: 4)
ATGCCGTTT
F8 CDR2 VL-
(SEQ ID NO: 5)
GGTGCATCCAGCAGGGCCACT
F8 CDR3 VL-
(SEQ ID NO: 6)
ATGCGTGGTCGGCCGCCG
Nucleotide sequence of F8-VH
(SEQ ID NO: 7)
GAGGTGCAGCTGTTGGAGTCTGGGGGAGGCTTGGTACAGCCTGGG
GGGTCCCTGAGACTCTCCTGTGCAGCCTCTGGATTCACCTTTAGC
CTGTTTACGATGAGCTGGGTCCGCCAGGCTCCAGGGAAGGGGCTG
GAGTGGGTCTCAGCTATTAGTGGTAGTGGTGGTAGCACATACTAC
GCAGACTCCGTGAAGGGCCGGTTCACCATCTCCAGAGACAATTCC
AAGAACACGCTGTATCTGCAAATGAACAGCCTGAGAGCCGAGGAC
ACGGCCGTATATTACTGTGCGAAAAGTACTCATTTGTATCTTTTT
GACTACTGGGGCCAGGGAACCCTGGTCACCGTCTCGAGT
Nucleotide sequence of F8-VL
(SEQ ID NO: 8)
GAAATTGTGTTGACGCAGTCTCCAGGCACCCTGTCTTTGTCTCCA
GGGGAAAGAGCCACCCTCTCCTGCAGGGCCAGTCAGAGTGTTAGC
ATGCCGTTTTTAGCCTGGTACCAGCAGAAACCTGGCCAGGCTCCC
AGGCTCCTCATCTATGGTGCATCCAGCAGGGCCACTGGCATCCCA
GACAGGTTCAGTGGCAGTGGGTCTGGGACAGACTTCACTCTCACC
ATCAGCAGACTGGAGCCTGAAGATTTTGCAGTGTATTACTGTCAG
CAGATGCGTGGTCGGCCGCCGACGTTCGGCCAAGGGACCAAGGTG
GAAATCAAA
Nucleotide sequence of the F8 diabody
(SEQ ID NO: 9)
GAGGTGCAGCTGTTGGAGTCTGGGGGAGGCTTGGTACAGCCTGGG
GGGTCCCTGAGACTCTCCTGTGCAGCCTCTGGATTCACCTTTAGC
CTGTTTACGATGAGCTGGGTCCGCCAGGCTCCAGGGAAGGGGCTG
GAGTGGGTCTCAGCTATTAGTGGTAGTGGTGGTAGCACATACTAC
GCAGACTCCGTGAAGGGCCGGTTCACCATCTCCAGAGACAATTCC
AAGAACACGCTGTATCTGCAAATGAACAGCCTGAGAGCCGAGGAC
ACGGCCGTATATTACTGTGCGAAAAGTACTCATTTGTATCTTTTT
GACTACTGGGGCCAGGGAACCCTGGTCACCGTCTCGAGTGGCGGT
AGCGGAGGGGAAATTGTGTTGACGCAGTCTCCAGGCACCCTGTCT
TTGTCTCCAGGGGAAAGAGCCACCCTCTCCTGCAGGGCCAGTCAG
AGTGTTAGCATGCCGTTTTTAGCCTGGTACCAGCAGAAACCTGGC
CAGGCTCCCAGGCTCCTCATCTATGGTGCATCCAGCAGGGCCACT
GGCATCCCAGACAGGTTCAGTGGCAGTGGGTCTGGGACAGACTTC
ACTCTCACCATCAGCAGACTGGAGCCTGAAGATTTTGCAGTGTAT
TACTGTCAGCAGATGCGTGGTCGGCCGCCGACGTTCGGCCAAGGG
ACCAAGGTGGAAATCAAA
The VH and VL domain CDRs of the F8 antibody
are shown in bold. The VH/VL domain linker
sequence is shown in bold and underlined.
Nucleotide sequence of F8- (murine) IL4
(SEQ ID NO: 10)
GAGGTGCAGCTGTTGGAGTCTGGGGGAGGCTTGGTACAGCCTGGG
GGGTCCCTGAGACTCTCCTGTGCAGCCTCTGGATTCACCTTTAGC
CTGTTTACGATGAGCTGGGTCCGCCAGGCTCCAGGGAAGGGGCTG
GAGTGGGTCTCAGCTATTAGTGGTAGTGGTGGTAGCACATACTAC
GCAGACTCCGTGAAGGGCCGGTTCACCATCTCCAGAGACAATTCC
AAGAACACGCTGTATCTGCAAATGAACAGCCTGAGAGCCGAGGAC
ACGGCCGTATATTACTGTGCGAAAAGTACTCATTTGTATCTTTTT
GACTACTGGGGCCAGGGAACCCTGGTCACCGTCTCGAGTGGCGGT
AGCGGAGGGGAAATTGTGTTGACGCAGTCTCCAGGCACCCTGTCT
TTGTCTCCAGGGGAAAGAGCCACCCTCTCCTGCAGGGCCAGTCAG
AGTGTTAGCATGCCGTTTTTAGCCTGGTACCAGCAGAAACCTGGC
CAGGCTCCCAGGCTCCTCATCTATGGTGCATCCAGCAGGGCCACT
GGCATCCCAGACAGGTTCAGTGGCAGTGGGTCTGGGACAGACTTC
ACTCTCACCATCAGCAGACTGGAGCCTGAAGATTTTGCAGTGTAT
TACTGTCAGCAGATGCGTGGTCGGCCGCCGACGTTCGGCCAAGGG
ACCAAGGTGGAAATCAAATCTTCCTCATCGGGTAGTAGCTCTTCC
GGCTCATCGTCCAGCGGCCATATCCACGGATGCGACAAAAATCAC
TTGAGAGAGATCATCGGCATTTTGAACGAGGTCACAGGAGAAGGG
ACGCCATGCACGGAGATGGATGTGCCAAACGTCCTCACAGCAACG
AAGAACACCACAGAGAGTGAGCTCGTCTGTAGGGCTTCCAAGGTG
CTTCGCATATTTTATTTAAAACATGGGAAAACTCCATGCTTGAAG
AAGAACTCTAGTGTTCTCATGGAGCTGCAGAGACTCTTTCGGGCT
TTTCGATGCCTGGATTCATCGATAAGCTGCACCATGAATGAGTCC
AAGTCCACATCACTGAAAGACTTCCTGGAAAGCCTAAAGAGCATC
ATGCAAATGGATTAGTGG
The VH and VL domain CDRs of the F8 antibody
are shown in bold. The VH/VL domain linker
sequence and the linker between the VL domain
and murine IL4 are shown in bold and
underlined. The sequence of murine IL4 is
based on NCBI reference sequence NM_021283.2
(GI:226874825) and is shown in italics.
NM_021283.2 covers the whole interleukin 4
sequence consisting of signal peptide (also
called leader sequence) and the protein. The
signal peptide is needed for expression but
is later on cleaved off. The mature protein
doesn't have these amino acids. For the
construction of F8-IL4 only the IL4 protein
encoding sequence was used as it was
appended to the F8 antibody and the signal
peptide used for expression of this fusion
protein was that of the antibody.
Nucleotide sequence of F8- (human) IL4
(SEQ ID NO: 11)
GAGGTGCAGCTGTTGGAGTCTGGGGGAGGCTTGGTACAGCCTGGG
GGGTCCCTGAGACTCTCCTGTGCAGCCTCTGGATTCACCTTTAGC
CTGTTTACGATGAGCTGGGTCCGCCAGGCTCCAGGGAAGGGGCTG
GAGTGGGTCTCAGCTATTAGTGGTAGTGGTGGTAGCACATACTAC
GCAGACTCCGTGAAGGGCCGGTTCACCATCTCCAGAGACAATTCC
AAGAACACGCTGTATCTGCAAATGAACAGCCTGAGAGCCGAGGAC
ACGGCCGTATATTACTGTGCGAAAAGTACTCATTTGTATCTTTTT
GACTACTGGGGCCAGGGAACCCTGGTCACCGTCTCGAGTGGCGGT
AGCGGAGGGGAAATTGTGTTGACGCAGTCTCCAGGCACCCTGTCT
TTGTCTCCAGGGGAAAGAGCCACCCTCTCCTGCAGGGCCAGTCAG
AGTGTTAGCATGCCGTTTTTAGCCTGGTACCAGCAGAAACCTGGC
CAGGCTCCCAGGCTCCTCATCTATGGTGCATCCAGCAGGGCCACT
GGCATCCCAGACAGGTTCAGTGGCAGTGGGTCTGGGACAGACTTC
ACTCTCACCATCAGCAGACTGGAGCCTGAAGATTTTGCAGTGTAT
TACTGTCAGCAGATGCGTGGTCGGCCGCCGACGTTCGGCCAAGGG
ACCAAGGTGGAAATCAAATCTTCCTCATCGGGTAGTAGCTCTTCC
GGCTCATCGTCCAGCGGCCACAAGTGCGATATCACCTTACAGGAG
ATCATCAAAACTTTGAACAGCCTCACAGAGCAGAAGACTCTGTGC
ACCGAGTTGACCGTAACAGACATCTTTGCTGCCTCCAAGAACACA
ACTGAGAAGGAAACCTTCTGCAGGGCTGCGACTGTGCTCCGGGAG
TTGTAGAGGGAGGATGAGAAGGAGAGTGGGTGGGTGGGTGGGAGT
GGAGAGGAGTTGGAGAGGGAGAAGGAGGTGATGGGATTGGTGAAA
GGGGTGGAGAGGAAGGTGTGGGGGGTGGGGGGCTTGAATTCCTGT
CCTGTGAAGGAAGCCAACCAGAGTACGTTGGAAAACTTCTTGGAA
AGGCTAAAGACGATCATGAGAGAGAAATATTCAAAGTGTTCGAGC
The VH/VL domain linker sequence and the
linker between the VL domain and human IL4
are shown in bold and underlined. The
sequence of human IL4 is shown in italics.
The sequence of human IL4 is based on NCBI
reference sequence NM_000589.3 (GI:391224448).
NM_000589.3 covers the whole human
interleukin 4 sequence consisting of a signal
peptide (also called leader sequence) and the
protein. The signal peptide is needed for
expression but is later on cleaved off. The
mature protein doesn't have these amino
acids. For the construction of F8-(human)IL4
only the IL4 protein encoding sequence is
used as it is be appended to the F8 antibody
and the signal peptide used for expression
of this fusion protein is therefore that of
the antibody.
Amino acid sequence of F8 CDR's
F8 CDR1 VH-
(SEQ ID NO: 12)
LFT
F8 CDR2 VH-
(SEQ ID NO: 13)
SGSGGS
F8 CDR3 VH-
(SEQ ID NO: 14)
STHLYL
F8 CDR1 VL-
(SEQ ID NO: 15)
MPF
F8 CDR2 VL-
(SEQ ID NO: 16)
GASSRAT
F8 CDR3 VL-
(SEQ ID NO: 17)
MRGRPP
Amino acid sequence of F8 VH
(SEQ ID NO: 18)
EVQLLESGGGLVQPGGSLRLSCAASGFTFSLFTMSWVRQAPGKGL
EWVSAISGSGGSTYYADSVKGRFTISRDNSKNTLYLQMNSLRAED
TAVYYCAKSTHLYLFDYWGQGTLVTVSS
Amino acid sequence of F8 VL
(SEQ ID NO: 19)
EIVLTQSPGTLSLSPGERATLSCRASQSVSMPFLAWYQQKPGQAP
RLLIYGASSRATGIPDRFSGSGSGTDFTLTISRLEPEDFAVYYCQ
QMRGRPPTFGQGTKVEIK
Amino acid sequence of the F8 diabody
(SEQ ID NO: 20)
EVQLLESGGGLVQPGGSLRLSCAASGFTFSLFTMSWVRQAPGKGL
EWVSAISGSGGSTYYADSVKGRFTISRDNSKNTLYLQMNSLRAED
TAVYYCAKSTHLYLFDYWGQGTLVTVSSGGSGGEIVLTQSPGTLS
LSPGERATLSCRASQSVSMPFLAWYQQKPGQAPRLLIYGASSRAT
GIPDRFSGSGSGTDFTLTISRLEPEDFAVYYCQQMRGRPPTFGQG
TKVEIK
The VH and VL domain CDRs of the F8 antibody
are shown in bold. The VH/VL domain linker
sequence is shown in bold and underlined.
Amino acid sequence of F8- (murine) IL4
(SEQ ID NO: 21)
EVQLLESGGGLVQPGGSLRLSCAASGFTFSLFTMSWVRQAPGKGL
EWVSAISGSGGSTYYADSVKGRFTISRDNSKNTLYLQMNSLRAED
TAVYYCAKSTHLYLFDYWGQGTLVTVSSGGSGGEIVLTQSPGTLS
LSPGERATLSCRASQSVSMPFLAWYQQKPGQAPRLLIYGASSRAT
GIPDRFSGSGSGTDFTLTISRLEPEDFAVYYCQQMRGRPPTFGQG
TKVEIKSSSSGSSSSGSSSSGHIHGCDKNHLREIIGILNEVTGEG
TPCTEMDVPNVLTATKNTTESELVCRASKVLRIFYLKHGKTPCLK
KNSSVLMELQRLFRAFRCLDSSISCTMNESKSTSLKDFLESLKSI
MQMDYS
The VH and VL domain CDRs of the F8 antibody
are shown in bold. The VH/VL domain linker
sequence and the linker between the VL domain
and IL4 are shown in bold and underlined. The
sequence of murine IL4 is based on
NM_021283.2 (GI:226874825) and is shown in
italics.
Amino acid sequence of F8- (human) IL4
(SEQ ID NO: 22)
EVQLLESGGGLVQPGGSLRLSCAASGFTFSLFTMSWVRQAPGKGL
EWVSAISGSGGSTYYADSVKGRFTISRDNSKNTLYLQMNSLRAED
TAVYYCAKSTHLYLFDYWGQGTLVTVSSGGSGGEIVLTQSPGTLS
LSPGERATLSCRASQSVSMPFLAWYQQKPGQAPRLLIYGASSRAT
GIPDRFSGSGSGTDFTLTISRLEPEDFAVYYCQQMRGRPPTFGQG
TKVEIKSSSSGSSSSGSSSSGHKCDITLQEIIKTLNSLTEQKTLC
TELTVTDIFAASKNTTEKETFCRAATVLRQFYSHHEKDTRCLGAT
AQQFHRHKQLIRFLKRLDRNLWGLAGLNSCPVKEANQSTLENFLE
RLKTIMREKYSKCSS
The VH/VL domain linker sequence and the
linker between the VL domain and IL4 are
shown in bold and underlined. The sequence of
human IL4 is based on NCBI Reference Sequence
NM_000589.3 (GI:391224448) and is shown in
italics
Amino acid sequence of F8 VH and VL domain
diabody linker
(SEQ ID NO: 23)
GGSGG
Amino acid sequence of F8 VL domain-IL4
linker sequence
(SEQ ID NO: 24)
SSSSGSSSSGSSSSG
Amino acid sequence of L19 CDR's
L19 CDR1 VH-
(SEQ ID NO: 25)
Ser Phe Ser Met Ser
L19 CDR2 VH-
(SEQ ID NO: 26)
Ser Ile Ser Gly Ser Ser Gly Thr Thr Tyr Tyr
Ala Asp Ser Val Lys
L19 CDR3 VH-
(SEQ ID NO: 27)
Pro Phe Pro Tyr Phe Asp Tyr
L19 CDR1 VL-
(SEQ ID NO: 28)
Arg Ala Ser Gln Ser Val Ser Ser Ser Phe Leu
Ala
L19 CDR2 VL-
(SEQ ID NO: 29)
Tyr Ala Ser Ser Arg Ala Thr
L19 CDR3 VL-
(SEQ ID NO: 30)
Gln Gln Thr Gly Arg Ile Pro Pro Thr
Amino acid sequence of L19 VH domain
(SEQ ID NO: 31)
Glu Val Gln Leu Leu Glu Ser Gly Gly Gly Leu
Val Gln Pro Gly Gly Ser Leu Arg Leu Ser Cys
Ala Ala Ser Gly Phe Thr Phe Ser Ser Phe Ser
Met Ser Trp Val Arg Gln Ala Pro Gly Lys Gly
Leu Glu Trp Val Ser Ser Ile Ser Gly Ser Ser
Gly Thr Thr Tyr Tyr Ala Asp Ser Val Lys Gly
Arg Phe Thr Ile Ser Arg Asp Asn Ser Lys Asn
Thr Leu Tyr Leu Gln Met Asn Ser Leu Arg Ala
Glu Asp Thr Ala Val Tyr Tyr Cys Ala Lys Pro
Phe Pro Tyr Phe Asp Tyr Trp Gly Gln Gly Thr
Leu Val Thr Val Ser Ser
Amino acid sequence of L19 VL domain
(SEQ ID NO: 32)
Glu Ile Val Leu Thr Gln Ser Pro Gly Thr Leu
Ser Leu Ser Pro Gly Glu Arg Ala Thr Leu Ser
Cys Arg Ala Ser Gln Ser Val Ser Ser Ser Phe
Leu Ala Trp Tyr Gln Gln Lys Pro Gly Gln Ala
Pro Arg Leu Leu Ile Tyr Tyr Ala Ser Ser Arg
Ala Thr Gly Ile Pro Asp Arg Phe Ser Gly Ser
Gly Ser Gly Thr Asp Phe Thr Leu Thr Ile Ser
Arg Leu Glu Pro Glu Asp Phe Ala Val Tyr Tyr
Cys Gln Gln Thr Gly Arg Ile Pro Pro Thr Phe
Gly Gln Gly Thr Lys Val Glu Ile Lys
Amino acid sequence of scFv(L19)
(SEQ ID NO: 33)
Glu Val Gln Leu Leu Glu Ser Gly Gly Gly Leu
Val Gln Pro Gly Gly Ser Leu Arg Leu Ser Cys
Ala Ala Ser Gly Phe Thr Phe Ser Ser Phe Ser
Met Ser Trp Val Arg Gln Ala Pro Gly Lys Gly
Leu Glu Trp Val Ser Ser Ile Ser Gly Ser Ser
Gly Thr Thr Tyr Tyr Ala Asp Ser Val Lys Gly
Arg Phe Thr Ile Ser Arg Asp Asn Ser Lys Asn
Thr Leu Tyr Leu Gln Met Asn Ser Leu Arg Ala
Glu Asp Thr Ala Val Tyr Tyr Cys Ala Lys Pro
Phe Pro Tyr Phe Asp Tyr Trp Gly Gln Gly Thr
Leu Val Thr Val Ser Ser Gly Asp Gly Ser Ser
Gly Gly Ser Gly Gly Ala Ser Glu Ile Val Leu
Thr Gln Ser Pro Gly Thr Leu Ser Leu Ser Pro
Gly Glu Arg Ala Thr Leu Ser Cys Arg Ala Ser
Gln Ser Val Ser Ser Ser Phe Leu Ala Trp Tyr
Gln Gln Lys Pro Gly Gln Ala Pro Arg Leu Leu
Ile Tyr Tyr Ala Ser Ser Arg Ala Thr Gly Ile
Pro Asp Arg Phe Ser Gly Ser Gly Ser Gly Thr
Asp Phe Thr Leu Thr Ile Ser Arg Leu Glu Pro
Glu Asp Phe Ala Val Tyr Tyr Cys Gln Gln Thr
Gly Arg Ile Pro Pro Thr Phe Gly Gln Gly Thr
Lys Val Glu Ile Lys
Amino acid sequence of F16 CDR's
F16 CDR1 VH-
(SEQ ID NO: 34)
RYGMS
F16 CDR2 VH-
(SEQ ID NO: 35)
AISGSGGSTYYADSVKG
F16 CDR3 VH-
(SEQ ID NO: 36)
AHNAFDY
F16 CDR1 VL-
(SEQ ID NO: 37)
QGDSLRSYYAS
F16 CDR2 VL-
(SEQ ID NO: 38)
GKNNRPS
F16 CDR3 VL-
(SEQ ID NO: 39)
NSSVYTMPPVV
Amino acid sequence F16 VH domain
(SEQ ID NO: 40)
EVQLLESGGGLVQPGGSLRLSCAASGFTFSRYGMSWVRQAPGKGL
EWVSAISGSGGSTYYADSVKGRFTISRDNSKNTLYLQMNSLRAED
TAVYYCAKAHNAFDYWGQGTLVTVSR
Amino acid sequence F16 VL domain
(SEQ ID NO: 41)
SSELTQDPAVSVALGQTVRITCQGDSLRSYYASWYQQKPGQAPVL
VIYGKNNRPSGIPDRFSGSSSGNTASLTITGAQAEDEADYYCNSS
VYTMPPVVFGGGTKLTVL
Amino acid sequence of the scFv(F16)
(SEQ ID NO: 42)
EVQLLESGGGLVQPGGSLRLSCAASGFTFSRYGMSWVRQAPGKGL
EWVSAISGSGGSTYYADSVKGRFTISRDNSKNTLYLQMNSLRAED
TAVYYCAKAHNAFDYWGQGTLVTVSRGGGSGGGSGGSSELTQDPA
VSVALGQTVRITCQGDSLRSYYASWYQQKPGQAPVLVIYGKNNRP
SGIPDRFSGSSSGNTASLTITGAQAEDEADYYCNSSVYTMPPVVF
GGGTKLTVL
The VH and VL domain linker sequence is shown
underlined
Nucleotide sequence of KSF-IL4
(SEQ ID NO: 43)
GAGGTGCAGCTGTTGGAGTCTGGGGGAGGCTTGGTACAGCCTGGG
GGGTCCCTGAGACTCTCCTGTGCAGCCTCTGGATTCACCTTTAGC
AGCTATGCCATGAGCTGGGTCCGCCAGGCTCCAGGGAAGGGGCTG
GAGTGGGTCTCAGCTATTAGTGGTAGTGGTGGTAGCACATACTAC
GCAGACTCCGTGAAGGGCCGGTTCACCATCTCCAGAGACAATTCC
AAGAACACGCTGTATCTGCAAATGAACAGCCTGAGAGCCGAAGAC
ACGGCCGTATATTACTGTGCGAAATCGCCTAAGGTGTCGCTTTTT
GACTACTGGGGCCAGGGAACCCTGGTCACCGTCTCGAGT [symbol]
TCTGAGCTGACTCAGGACCCTGCTGTG
TCTGTGGCCTTGGGACAGACAGTCAGGATCACATGCCAAGGAGAC
AGTCTCAGAAGCTATTATGCAAGCTGGTACCAGCAGAAGCCAGGA
CAGGCCCCTGTACTTGTCATCTATGGTAAAAACAACCGGCCCTCA
GGGATCCCAGACCGATTCTCTGGCTCCAGCTCAGGAAACACAGCT
TCCTTGACCATCACTGGGGCTCAGGCGGAAGATGAGGCTGACTAT
TACTGTAACTCCTCTCCCCTGAATCGGCTGGCTGTGGTATTCGGC
GGAGGGACCAAGCTGACCGTCCTAGGCTCTTCCTCATCGGGTAGT
AGCTCTTCCGGCTCATCGTCCAGCGGCCATATCCACGGATGCGAC
AAAAATCACTTGAGAGAGATCATCGGCATTTTGAACGAGGTCACA
GGAGAAGGGACGCCATGCACGGAGATGGATGTGCCAAACGTCCTC
ACAGCAACGAAGAACACCACAGAGAGTGAGCTCGTCTGTAGGGCT
TCCAAGGTGCTTCGCATATTTTATTTAAAACATGGGAAAACTCCA
TGCTTGAAGAAGAACTCTAGTGTTCTCATGGAGCTGCAGAGACTC
TTTCGGGCTTTTCGATGCCTGGATTCATCGATAAGCTGCACCATG
AATGAGTCCAAGTCCACATCACTGAAAGACTTCCTGGAAAGCCTA
AAGAGCATCATGCAAATGGATTACTCG
The VH/VL domain linker sequence and the
linker between the VL domain and human IL4
are shown in bold. The sequence of human IL4
is shown in italics.
Amino acid sequence of KSF-murine IL4
(SEQ ID NO: 44)
EVQLLESGGGLVQPGGSLRLSCAASGFTFSSYAMSWVRQAPGKGL
EWVSAISGSGGSTYYADSVKGRFTISRDNSKNTLYLQMNSLRAED
TAVYYCAKSPKVSLFDYWGQGTLVTVSSGGSGGSELTQDPAVSVA
LGQTVRITCQGDSLRSYYASWYQQKPGQAPVLVIYGKNNRPSGIP
DRFSGSSSGNTASLTITGAQAEDEADYYCNSSPLNRLAVVFGGGT
KLTVLGSSSSGSSSSGSSSSGHIHGCDKNHLREIIGILNEVTGEG
TPCTEMDVPNVLTATKNTTESELVCRASKVLRIFYLKHGKTPCLK
KNSSVLMELQRLFRAFRCLDSSISCTMNESKSTSLKDFLESLKSI
MQMDYS
The VH/VL domain linker sequence and the
linker between the VL domain and human IL4
are shown in bold. The sequence of IL4 is
shown in italics.
Nucleotide sequence of TNFR-Fc
(SEQ ID NO: 45)
GTGCCCGCCCAGGTTGTOTTGACACCCTACAAACCGGAACCTGGG
TACGAGTGCCAGATCTCACAGGAATACTATGACAGGAAGGCTCAG
ATGTGCTGTGCTAAGTGTCCTCCTGGCCAATATGTGAAACATTTC
TGCAACAAGACCTOGGACACCGTGTGTGCGGACTGTGAGGCAAGC
ATGTATACCCAGGTCTGGAACCAGTTTCGTACATGTTTGAGCTGC
AGTTCTTCCTGTACCACTGACCAGGTGGAGATCCGCGCCTGCACT
AAACAGCAGAACCGAGTGTGTGCTTGCGAAGCTGGCAGGTACTGC
GCCTTGAAAACCCATTCTGGCAGCTGTCGACAGTGCATGAGGCTG
AGCAAGTGCGGCCCTGGCTTCGGAGTGGCCAGTTCAAGAGCCCCA
AATGGAAATGTGCTATGCAAGGCCTGTGCCCCAGGGACGTTCTCT
GACACCACATCATCCACTGATGTGTGCAGGCCCCACCGCATCTGT
AGCATCCTGGCTATTCCCGGAAATGCAAGCACAGATGCAGTCTGT
GCGCCCGAGTCCCCAACTCTAAGTGCCATCCCAAGGACACTCTAC
GTATCTCAGCCAGAGCCCACAAGATCCCAACCCCTGGATCAAGAG
CCAGGGCCCAGCCAAACTCCAAGCATCCTTACATCGTTGGGTTCA
ACCCCCATTATTGAACAAAGTACCAAGGGTGGCGTGCCCAGGGAT
TGTGGTTGTAAGCCTTGCATATGTACAGTCCCAGAAGTATCATCT
GTCTTCATCTTCCCCCCAAAGCCCAAGGATGTGCTCACCATTACT
CTGACTCCTAAGGTCACGTGTGTTGTGGTAGACATCAGCAAGGAT
GATCCCGAGGTCCAGTTCAGCTGGTTTGTAGATGATGTGGAGGTG
CACACAGCTCAGACAAAACCCCGGGAGGAGCAGTTCAACAGCACT
TTCCGTTCAGTCAGTGAACTTCCCATCATGCACCAGGACTGGCTC
AATGGCAAGGAGTTCAAATGCAGGGTCAACAGTGCAGCTTTCCCT
GCCCCCATCGAGAAAACCATCTCCAAAACCAAAGGCAGACCGAAG
GCTCCACAGGTGTACACCATTCCACCTCCCAAGGAGCAGATGGCC
AAGGATAAAGTCAGTCTGACCTGCATGATAACAGACTTCTTCCCT
GAAGACATTACTGTGGAGTGGCAGTGGAATGGGCAGCCAGCGGAG
AACTACAAGAACACTCAGCCCATCATGGACACAGATGGCTCTTAC
TTCGTCTACAGCAAGCTCAATGTGCAGAAGAGCAACTGGGAGGCA
GGAAATACTTTCACCTGCTCTGTGTTACATGAGGGCCTGCACAAC
CACCATACTGAGAAGAGCCTCTCCCACTCTCCTGGTAAA
Signal peptide. The extracellular domain of
TNFRII is underlined. The hinge region is
shown in bold. The CH2 is shown in italics.
The CH3 is shown in bold and underlined.
Amino acid sequence of TNFR-Fc
(SEQ ID NO: 46)
VPAQVVLTPYKPEPGYECQISQEYYDRKAQMCCAKCPPGQYVKHF
CNKTSDTVCADCEASMYTQVWNQFRTCLSCSSSCTTDQVEIRACT
KQQNRVCACEAGRYCALKTHSGSCRQCMRLSKCGPGFGVASSRAP
NGNVLCKACAPGTFSDTTSSTDVCRPHRICSILAIPGNASTDAVC
APESPTLSAIPRTLYVSQPEPTRSQPLDQEPGPSQTPSILTSLGS
TPIIEQSTKGGVPRDCGCKPCICTVPEVSSVFIFPPKPKDVLTIT
LTPKVTCVVVDISKDDPEVQFSWFVDDVEVHTAQTKPREEQFNST
FRSVSELPIMHQDWLNGKEFKCRVNSAAFPAPIEKTISKTKGRPK
APQVYTIPPPKEQMAKDKVSLTCMITDFFPEDITVEWQWNGQPAE
NYKNTQPIMDTDGSYFVYSKLNVQKSNWEAGNTFTCSVLHEGLHN
HHTEKSLSHSPGK
Nucleotide sequence of (murine)IL12-F8-F8
(SEQ ID NO: 47)
ATGTGGGAGCTGGAGAAAGACGTTTATGTTGTAGAGGTGGACTGG
ACTCCCGATGCCCCTGGAGAAACAGTGAACCTCACCTGTGACACG
CCTGAAGAAGATGACATCACCTGGACCTCAGACCAGAGACATGGA
GTCATAGGCTCTGGAAAGACCCTGACCATCACTGTCAAAGAGTTT
CTAGATGCTGGCCAGTACACCTGCCACAAAGGAGGCGAGACTCTG
AGCCACTCACATCTGCTGCTCCACAAGAAGGAAAATGGAATTTGG
TCCACTGAAATTTTAAAAAATTTCAAAAACAAGACTTTCCTGAAG
TGTGAAGCACCAAATTACTCCGGACGGTTCACGTGCTCATGGCTG
GTGCAAAGAAACATGGACTTGAAGTTCAACATCAAGAGCAGTAGC
AGTTCCCCTGACTCTCGGGCAGTGACATGTGGAATGGCGTCTCTG
TCTGCAGAGAAGGTCACACTGGACCAAAGGGACTATGAGAAGTAT
TCAGTGTCCTGCCAGGAGGATGTCACCTGCCCAACTGCCGAGGAG
ACCCTGCCCATTGAACTGGCGTTGGAAGCACGGCAGCAGAATAAA
TATGAGAACTACAGCACCAGCTTCTTCATCAGGGACATCATCAAA
CCAGACCCGCCCAAGAACTTGCAGATGAAGCCTTTGAAGAACTCA
CAGGTGGAGGTCAGCTGGGAGTACCCTGACTCCTGGAGCACTCCC
CATTCCTACTTCTCCCTCAAGTTCTTTGTTCGAATCCAGCGCAAG
AAAGAAAAGATGAAGGAGACAGAGGAGGGGTGTAACCAGAAAGGT
GCGTTCCTCGTAGAGAAGACATCTACCGAAGTCCAATGCAAAGGC
GGGAATGTCTGCGTGCAAGCTCAGGATCGCTATTACAATTCCTCG
TGCAGCAAGTGGGCATGTGTTCCCTGCAGGGTCCGATCCGGTGGA
GGCGGTTCAGGCGGAGGTGGCTCTGGCGGTGGCGGATCG

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

GGTAGCGCTGATGGAGGTGAGGTGCAGCTGTTGGAGTCTGGGGGA
GGCTTGGTACAGCCTGGGGGGTCCCTGAGACTCTCCTGTGCAGCC
TCTGGATTCACCTTTAGCCTGTTTACGATGAGCTGGGTCCGCCAG
GCTCCAGGGAAGGGGCTGGAGTGGGTCTCAGCTATTAGTGGTAGT
GGTGGTAGCACATACTACGCAGACTCCGTGAAGGGCCGGTTCACC
ATCTCCAGAGACAATTCCAAGAACACGCTGTATCTGCAAATGAAC
AGCCTGAGAGCCGAGGACACGGCCGTATATTACTGTGCGAAAAGT
ACTCATTTGTATCTTTTTGACTACTGGGGCCAGGGAACCCTGGTC
ACCGTCTCGAGTGGCGGTAGCGGAGGGGAAATTGTGTTGACGCAG
TCTCCAGGCACCCTGTCTTTGTCTCCAGGGGAAAGAGCCACCCTC
TCCTGCAGGGCCAGTCAGAGTGTTAGCATGCCGTTTTTAGCCTGG
TACCAGCAGAAACCTGGCCAGGCTCCCAGGCTCCTCATCTATGGT
GCATCCAGCAGGGCCACTGGCATCCCAGACAGGTTCAGTGGCAGT
GGGTCTGGGACAGACTTCACTCTCACCATCAGCAGACTGGAGCCT
GAAGATTTTGCAGTGTATTACTGTCAGCAGATGCGTGGTCGGCCG
CCGACGTTCGGCCAAGGGACCAAGGTGGAAATCAAATCTTCCTCA
TCCGGAAGTAGCTCTTCGGGATCCTCGTCCAGCGGCGAGGTGCAG
CTGTTGGAGTCTGGGGGAGGCTTGGTACAGCCTGGGGGGTCCCTG
AGACTCTCCTGTGCAGCCTCTGGATTCACCTTTAGCCTGTTTACG
ATGAGCTGGGTCCGCCAGGCTCCAGGGAAGGGGCTGGAGTGGGTC
TCAGCTATTAGTGGTAGTGGTGGTAGCACATACTACGCAGACTCC
GTGAAGGGCCGGTTCACCATCTCCAGAGACAATTCCAAGAACACG
CTGTATCTGCAAATGAACAGCCTGAGAGCCGAGGACACGGCCGTA
TATTACTGTGCGAAAAGTACTCATTTGTATCTTTTTGACTACTGG
GGCCAGGGAACCCTGGTCACCGTCTCGAGTGGCGGTAGCGGAGGG
GAAATTGTGTTGACGCAGTCTCCAGGCACCCTGTCTTTGTCTCCA
GGGGAAAGAGCCACCCTCTCCTGCAGGGCCAGTCAGAGTGTTAGC
ATGCCGTTTTTAGCCTGGTACCAGCAGAAACCTGGCCAGGCTCCC
AGGCTCCTCATCTATGGTGCATCCAGCAGGGCCACTGGCATCCCA
GACAGGTTCAGTGGCAGTGGGTCTGGGACAGACTTCACTCTCACC
ATCAGCAGACTGGAGCCTGAAGATTTTGCAGTGTATTACTGTCAG
CAGATGCGTGGTCGGCCGCCGACGTTCGGCCAAGGGACCAAGGTG
GAAATCAAA
The VH/VL domain linker sequence, the linker
between murine p40 and murine p35, the linker
between p35 and the antibody and the linker
between the F8 antibodies are shown in bold
and underlined. Murine p40 is shown in
italics and p35 is shown in italics and bold.
Amino acid sequence of (murine)IL12-F8-F8
(SEQ ID NO: 48)
MWELEKDVYVVEVDWTPDAPGETVNLTCDTPEEDDITWTSDQRHG
VIGSGKTLTITVKEFLDAGQYTCHKGGETLSHSHLLLHKKENGIW
STEILKNFKNKTFLKCEAPNYSGRFTCSWLVQRNMDLKFNIKSSS
SSPDSRAVTCGMASLSAEKVTLDQRDYEKYSVSCQEDVTCPTAEE
TLPIELALEARQQNKYENYSTSFFIRDIIKPDPPKNLQMKPLKNS
QVEVSWEYPDSWSTPHSYFSLKFFVRIQRKKEKMKETEEGCNQKG
AFLVEKTSTEVQCKGGNVCVQAQDRYYNSSCSKWACVPCRVRSGG
GGSGGGGSGGGGS

[symbol]

[symbol]

[symbol]

[symbol]

[symbol]

[symbol]

[symbol]

SADGGEVQLLESGGGLVQPGGSLRLSCAASGFTFSLFTMSWVRQA
PGKGLEWVSAISGSGGSTYYADSVKGRFTISRDNSKNTLYLQMNS
LRAEDTAVYYCAKSTHLYLFDYWGQGTLVTVSSGGSGGEIVLTQS
PGTLSLSPGERATLSCRASQSVSMPFLAWYQQKPGQAPRLLIYGA
SSRATGIPDRFSGSGSGTDFTLTISRLEPEDFAVYYCQQMRGRPP
TFGQGTKVEIKSSSSGSSSSGSSSSGEVQLLESGGGLVQPGGSLR
LSCAASGFTFSLFTMSWVRQAPGKGLEWVSAISGSGGSTYYADSV
KGRFTISRDNSKNTLYLQMNSLRAEDTAVYYCAKSTHLYLFDYWG
QGTLVTVSSGGSGGEIVLTQSPGTLSLSPGERATLSCRASQSVSM
PFLAWYQQKPGQAPRLLIYGASSRATGIPDRFSGSGSGTDFTLTI
SRLEPEDFAVYYCQQMRGRPPTFGQGTKVEIK
The linker between murine p40 and murine p35,
the linker between p35 and the first F8
antibody and the linker between the F8
antibodies are shown in bold and underlined.
Murine p40 is shown in italics and p35 is
shown in italics and bold.
Nucleotide sequence of F8-(human)IL2
(SEQ ID NO: 49)
GAGGTGCAGCTGTTGGAGTCTGGGGGAGGCTTGGTACAGCCTGGG
GGGTCCCTGAGACTCTCCTGTGCAGCCTCTGGATTCACCTTTAGC
CTGTTTACGATGAGCTGGGTCCGCCAGGCTCCAGGGAAGGGGCTG
GAGTGGGTCTCAGCTATTAGTGGTAGTGGTGGTAGCACATACTAC
GCAGACTCCGTGAAGGGCCGGTTCACCATCTCCAGAGACAATTCC
AAGAACACGCTGTATCTGCAAATGAACAGCCTGAGAGCCGAGGAC
ACGGCCGTATATTACTGTGCGAAAAGTACTCATTTGTATCTTTTT
GACTACTGGGGCCAGGGAACCCTGGTCACCGTCTCGAGTGGCGGT
AGCGGAGGGGAAATTGTGTTGACGCAGTCTCCAGGCACCCTGTCT
TTGTCTCCAGGGGAAAGAGCCACCCTCTCCTGCAGGGCCAGTCAG
AGTGTTAGCATGCCGTTTTTAGCCTGGTACCAGCAGAAACCTGGC
CAGGCTCCCAGGCTCCTCATCTATGGTGCATCCAGCAGGGCCACT
GGCATCCCAGACAGGTTCAGTGGCAGTGGGTCTGGGACAGACTTC
ACTCTCACCATCAGCAGACTGGAGCCTGAAGATTTTGCAGTGTAT
TACTGTCAGCAGATGCGTGGTCGGCCGCCGACGTTCGGCCAAGGG
ACCAAGGTGGAAATCAAATCTTCCTCATCGGGTAGTAGCTCTTCC
GGCTCATCGTCCAGCGGCGCACCTACTTCAAGTTCTACAAAGAAA
ACACAGCTACAACTGGAGCATTTACTGCTGGATTTACAGATGATT
TTGAATGGAATTAATAATTACAAGAATCCCAAACTCACCAGGATG
CTCACATTTAAGTTTTACATGCCCAAGAAGGCCACAGAACTGAAA
CATCTTCAGTGTCTAGAAGAAGAACTCAAACCTCTGGAGGAAGTG
CTAAATTTAGCTCAAAGCAAAAACTTTCACTTAAGACCCAGGGAC
TTAATCAGCAATATCAACGTAATAGTTCTGGAACTAAAGGGATCT
GAAACAACATTCATGTGTGAATATGCTGATGAGACAGCAACCATT
GTAGAATTTCTGAACAGATGGATTACCTTTTGTCAAAGCATCATC
TCAACACTGACT
Amino acid sequence of F8-(human)IL2
(SEQ ID NO: 50)
EVQLLESGGGLVQPGGSLRLSCAASGFTFSLFTMSWVRQAPGKGL
EWVSAISGSGGSTYYADSVKGRFTISRDNSKNTLYLQMNSLRAED
TAVYYCAKSTHLYLFDYWGQGTLVTVSSGGSGGEIVLTQSPGTLS
LSPGERATLSCRASQSVSMPFLAWYQQKPGQAPRLLIYGASSRAT
GIPDRFSGSGSGTDFTLTISRLEPEDFAVYYCQQMRGRPPTFGQG
TKVEIKSSSSGSSSSGSSSSGAPTSSSTKKTQLQLEHLLLDLQMI
LNGINNYKNPKLTRMLTFKFYMPKKATELKHLQCLEEELKPLEEV
LNLAQSKNFHLRPRDLISNINVIVLELKGSETTFMCEYADETATI
VEFLNRWITFCQSIISTLT
The linker between the VH domain and human
IL2 is shown in bold. The sequence of human
IL2 is shown in italics.
Nucleotide sequence of murine IL4 (based on
NM_021283.2; GI:226874825)
(SEQ ID NO: 51)
CATATCCACGGATGCGACAAAAATCACTTGAGAGAGATCATCGGC
ATTTTGAACGAGGTCACAGGAGAAGGGACGCCATGCACGGAGATG
GATGTGCCAAACGTCCTCACAGCAACGAAGAACACCACAGAGAGT
GAGCTCGTCTGTAGGGCTTCCAAGGTGCTTCGCATATTTTATTTA
AAACATGGGAAAACTCCATGCTTGAAGAAGAACTCTAGTGTTCTC
ATGGAGCTGCAGAGACTCTTTCGGGCTTTTCGATGCCTGGATTCA
TCGATAAGCTGCACCATGAATGAGTCCAAGTCCACATCACTGAAA
GACTTCCTGGAAAGCCTAAAGAGCATCATGCAAATGGATTACTCG
Amino acid sequence of murine IL4 (based on
NM_021283.2; GI:226874825)
(SEQ ID NO: 52)
HIHGCDKNHLREIIGILNEVTGEGTPCTEMDVPNVLTATKNTTES
ELVCRASKVLRIFYLKHGKTPCLKKNSSVLMELQRLFRAFRCLDS
SISCTMNESKSTSLKDFLESLKSIMQMDYS
Nucleotide sequence of human IL4
(SEQ ID NO: 53)
TGCATCGTTAGCTTCTCCTGATAAACTAATTGCCTCACATTGTCA
CTGCAAATCGACACCTATTAATGGGTCTCACCTCCCAACTGCTTC
CCCCTCTGTTCTTCCTGCTAGCATGTGCCGGCAACTTTGTCCACG
GACACAAGTGCGATATCACCTTACAGGAGATCATCAAAACTTTGA
ACAGCCTCACAGAGCAGAAGACTCTGTGCACCGAGTTGACCGTAA
CAGACATCTTTGCTGCCTCCAAGAACACAACTGAGAAGGAAACCT
TCTGCAGGGCTGCGACTGTGCTCCGGCAGTTCTACAGCCACCATG
AGAAGGACACTCGCTGCCTGGGTGCGACTGCACAGCAGTTCCACA
GGCACAAGCAGCTGATCCGATTCCTGAAACGGCTCGACAGGAACC
TCTGGGGCCTGGCGGGCTTGAATTCCTGTCCTGTGAAGGAAGCCA
ACCAGAGTACGTTGGAAAACTTCTTGGAAAGGCTAAAGACGATCA
TGAGAGAGAAATATTCAAAGTGTTCGAGCTGAATATTTTAATTTA
TGAGTTTTTGATAGCTTTATTTTTTAAGTATTTATATATTTATAA
CTCATCATAAAATAAAGTATATATAGAATCTAAAAAAAAAAAAAA
AAAAAAAAAAAA
The nucleotide sequence of human IL4 is based
on NCBI reference sequence number NM_000589.3
(GI:3912244481)
Amino acid sequence of human IL4
(SEQ ID NO: 54)
HKCDITLQEIIKTLNSLTEQKTLCTELTVTDIFAASKNTTEKETF
CRAATVLRQFYSHHEKDTRCLGATAQQFHRHKQLIRFLKRLDRNL
WGLAGLNSCPVKEANQSTLENFLERLKTIMREKYSKCSS
The amino acid sequence of human IL4 is based
on NCBI reference sequence number NM_000589.3
(GI:3912244481)
Nucleotide sequence of human IL2
(SEQ ID NO: 55)
GCACCTACTTCAAGTTCTACAAAGAAAACACAGCTACAACTGGAG
CATTTACTGCTGGATTTACAGATGATTTTGAATGGAATTAATAAT
TACAAGAATCCCAAACTCACCAGGATGCTCACATTTAAGTTTTAC
ATGCCCAAGAAGGCCACAGAACTGAAACATCTTCAGTGTCTAGAA
GAAGAACTCAAACCTCTGGAGGAAGTGCTAAATTTAGCTCAAAGC
AAAAACTTTCACTTAAGACCCAGGGACTTAATCAGCAATATCAAC
GTAATAGTTCTGGAACTAAAGGGATCTGAAACAACATTCATGTGT
GAATATGCTGATGAGACAGCAACCATTGTAGAATTTCTGAACAGA
TGGATTACCTTTTGTCAAAGCATCATCTCAACACTGACT
Amino acid sequence of human IL2
(SEQ ID NO: 56)
APTSSSTKKTQLQLEHLLLDLQMILNGINNYKNPKLTRMLTFKFY
MPKKATELKHLQCLEEELKPLEEVLNLAQSKNFHLRPRDLISNIN
VIVLELKGSETTFMCEYADETATIVEFLNRWITFCQSIISTLT
Nucleotide sequence of human IL12
(SEQ ID NO: 57)
ATATGGGAACTGAAGAAAGATGTTTATGTCGTAGAATTGGATTGG
TA [symbol] TGCCCCTGGAGAAATGGTGGTCCTCACCTGTGACAC
CCCTGAAGAAGATGGTATCACCTGGACCTTGGACCAGAGCAGTGA
GGTCTTAGGCTCTGGCAAAACCCTGACCATCCAAGTCAAAGAGTT
TGGAGATGCTGGCCAGTACACCTGTCACAAAGGAGGCGAGGTTCT
AAGCCATTCGCTCCTGCTGCTTCACAAAAAGGAAGATGGAATTTG
GTCCACTGATATTTTAAAGGACCAGAAAGAACCCAAAAATAAGAC
CTTTCTAAGATGCGAGGCCAAGAATTATTCTGGACGTTTCACCTG
CTGGTGGCTGACGACAATCAGTACTGATTTGACATTCAGTGTCAA
AAGCAGCAGAGGCTCTTCTGACCCCCAAGGGGTGACGTGCGGAGC
TGCTACACTCTCTGCAGAGAGAGTCAGAGGGGACAACAAGGAGTA
TGAGTACTCAGTGGAGTGCCAGGAGGACAGTGCCTGCCCAGCTGC
TGAGGAGAGTCTGCCCATTGAGGTCATGGTGGATGCCGTTCACAA
GCTCAAGTATGAAAACTACACCAGCAGCTTCTTCATCAGGGACAT
CATCAAACCTGACCCACCCAAGAACTTGCAGCTGAAGCCATTAAA
GAATTCTCGGCAGGTGGAGGTCAGCTGGGAGTACCCTGACACCTG
GAGTACTCCACATTCCTACTTCTCCCTGACATTCTGCGTTCAGGT
CCAGGGCAAGAGCAAGAGAGAAAAGAAAGATAGAGTCTTCACGGA
CAAGACCTCAGCCACGGTCATCTGCCGCAAAAATGCCAGCATTAG
CGTGCGGGCCCAGGACCGCTACTATAGCTCATCTTGGAGCGAATG
GGCATCTGTGCCCTGCAGTGGTGGAGGCGGTTCAGGCGGAGGTGG
CTCTGGCGGTGGCGGATCG

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

The sequence coding for the linker sequence,
(GGGGS)₃, between the p40 and p35 subunits of
IL12 is shown in bold and underlined. p40 is
shown in italics and p35 is shown in italics
and bold.
Amino acid sequence of human IL12
(SEQ ID NO: 58)
IWELKKDVYVVELDWYPDAPGEMVVLTCDTPEEDGITWTLDQSSE
VLGSGKTLTIQVKEFGDAGQYTCHKGGEVLSHSLLLLHKKEDGIW
STDILKDQKEPKNKTFLRCEAKNYSGRFTCWWLTTISTDLTFSVK
SSRGSSDPQGVTCGAATLSAERVRGDNKEYEYSVECQEDSACPAA
EESLPIEVMVDAVHKLKYENYTSSFFIRDIIKPDPPKNLQLKPLK
NSRQVEVSWEYPDTWSTPHSYFSLTFCVQVQGKSKREKKDRVFTD
KTSATVICRKNASISVRAQDRYYSSSWSEWASVPCSGGGGSGGGG
SGGGGSRNLPVATPDPGMFPCLHHSQNLLRAVSNMLQKARQTLEF
YPCTSEEIDHEDITKDKTSTVEACLPLELTKNESCLNSRETSFIT
NGSCLASRKTSFMMALCLSSIYEDLKMYQVEFKTMNAKLLMDPKR
QIFLDQNMLAVIDELMQALNFNSETVPQKSSLEEPDFYKTKIKLC
ILLHAFRIRAVTIDRVMSYLNAS
The linker sequence, (GGGGS)₃, between the
p40 and p35 subunits of IL12 is shown in bold
and underlined
Amino acid sequence of L19 diabody
(SEQ ID NO: 59)
EVQLLESGGGLVQPGGSLRLSCAASGFTFSSFSMSWVRQAPGKGL
EWVSSISGSSGTTYYADSVKGRFTISRDNSKNTLYLQMNSLRAED
TAVYYCAKPFPYFDYWGQGTLVTVSSGSSGGEIVLTQSPGTLSLS
PGERATLSCRASQSVSSSFLAWYQQKPGQAPRLLIYYASSRATGI
PDRFSGSGSGTDFTLTISRLEPEDFAVYYCQQTGRIPPTFGQGTK
VEIK
The VH/VL domain linker sequence is shown in
bold and underlined.
Amino acid sequence of F16 diabody
(SEQ ID NO: 60)
EVQLLESGGGLVQPGGSLRLSCAASGFTFSRYGMSWVRQAPGKGL
EWVSAISGSGGSTYYADSVKGRFTISRDNSKNTLYLQMNSLRAED
TAVYYCAKAHNAFDYWGQGTLVTVSRGGSGGSSELTQDPAVSVAL
GQTVRITCQGDSLRSYYASWYQQKPGQAPVLVIYGKNNRPSGIPD
RFSGSSSGNTASLTITGAQAEDEADYYCNSSVYTMPPVVFGGGTK
LTVL
The VH/VL domain linker sequence is
underlined.
Amino acid sequence of (human) IL12-F8-F8
(SEQ ID NO: 61)
IWELKKDVYVVELDWYPDAPGEMVVLTCDTPEEDGITWTLDQSSE
VLGSGKTLTIQVKEFGDAGQYTCHKGGEVLSHSLLLLHKKEDGIW
STDILKDQKEPKNKTFLRCEAKNYSGRFTCWWLTTISTDLTFSVK
SSRGSSDPQGVTCGAATLSAERVRGDNKEYEYSVECQEDSACPAA
EESLPIEVMVDAVHKLKYENYTSSFFIRDIIKPDPPKNLQLKPLK
NSRQVEVSWEYPDTWSTPHSYFSLTFCVQVQGKSKREKKDRVFTD
KTSATVICRKNASISVRAQDRYYSSSWSEWASVPCSGGGGSGGGG
SGGGGSRNLPVATPDPGMFPCLHHSQNLLRAVSNMLQKARQTLEF
YPCTSEEIDHEDITKDKTSTVEACLPLELTKNESCLNSRETSFIT
NGSCLASRKTSFMMALCLSSIYEDLKMYQVEFKTMNAKLLMDPKR
QIFLDQNMLAVIDELMQALNFNSETVPQKSSLEEPDFYKTKIKLC
ILLHAFRIRAVTIDRVMSYLNASGSADGGEVQLLESGGGLVQPGG
SLRLSCAASGFTFSLFTMSWVRQAPGKGLEWVSAISGSGGSTYYA
DSVKGRFTISRDNSKNTLYLQMNSLRAEDTAVYYCAKSTHLYLFD
YWGQGTLVTVSSGGSGGEIVLTQSPGTLSLSPGERATLSCRASQS
VSMPFLAWYQQKPGQAPRLLIYGASSRATGIPDRFSGSGSGTDFT
LTISRLEPEDFAVYYCQQMRGRPPTFGQGTKVEIKSSSSGSSSSG
SSSSGEVQLLESGGGLVQPGGSLRLSCAASGFTFSLFTMSWVRQA
PGKGLEWVSAISGSGGSTYYADSVKGRFTISRDNSKNTLYLQMNS
LRAEDTAVYYCAKSTHLYLFDYWGQGTLVTVSSGGSGGEIVLTQS
PGTLSLSPGERATLSCRASQSVSMPFLAWYQQKPGQAPRLLIYGA
SSRATGIPDRFSGSGSGTDFTLTISRLEPEDFAVYYCQQMRGRPP
TFGQGTKVEIK
Amino acid sequence of L19 diabody
(SEQ ID NO: 62)
EVQLLESGGGLVQPGGSLRLSCAASGFTFSSFSMSWVRQAPGKGL
EWVSSISGSSGTTYYADSVKGRFTISRDNSKNTLYLQMNSLRAED
TAVYYCAKPFPYFDYWGQGTLVTVSSGGSGGEIVLTQSPGTLSLS
PGERATLSCRASQSVSSSFLAWYQQKPGQAPRLLIYYASSRATGI
PDRFSGSGSGTDFTLTISRLEPEDFAVYYCQQTGRIPPTFGQGTK
VEIK
The VH/VL domain linker sequence is shown in
bold and underlined.
Amino acid sequence of the L19-IL10 conjugate
used in the RA experiments
(SEQ ID NO: 63)
EVQLLESGGGLVQPGGSLRLSCAASGFTFSSFSMSWVRQAPGKGL
EWVSSISGSSGTTYYADSVKGRFTISRDNSKNTLYLQMNSLRAED
TAVYYCAKPFPYFDYWGQGTLVTVSSGSSGGEIVLTQSPGTLSLS
PGERATLSCRASQSVSSSFLAWYQQKPGQAPRLLIYYASSRATGI
PDRFSGSGSGTDFTLTISRLEPEDFAVYYCQQTGRIPPTFGQGTK
VEIKSSSSGSSSSGSSSSGSPGQGTQSENSCTHFPGNLPNMLRDL
RDAFSRVKTFFQMKDQLDNLLLKESLLEDFKGYLGCQALSEMIQF
YLEEVMPQAENQDPDIKAHVNSLGENLKTLRLRLRRCHRFLPCEN
KSKAVEOVKNAFNKLQEKGIYKAMSEFDIFINYIEAYMTMKIRN
The VH/VL domain linker sequence and the
linker between the VL domain and IL10 are
shown in bold. The sequence of IL10 is shown
in italics.
Amino acid sequence of the L19-murine IL4
conjugate
(SEQ ID NO: 64)
EVQLLESGGGLVQPGGSLRLSCAASGFTFSSFSMSWVRQAPGKGL
EWVSSISGSSGTTYYADSVKGRFTISRDNSKNTLYLQMNSLRAED
TAVYYCAKPFPYFDYWGQGTLVTVSSGGSGGEIVLTQSPGTLSLS
PGERATLSCRASQSVSSSFLAWYQQKPGQAPRLLIYYASSRATGI
PDRFSGSGSGTDFTLTISRLEPEDFAVYYCQQTGRIPPTFGQGTK
VEIKSSSSGSSSSGSSSSGHIHGCDKNHLREIIGILNEVTGEGTP
CTEMDVPNVLTATKNTTESELVCRASKVLRIFYLKHGKTPCLKKN
SSVLMELQRLFRAFRCLDSSISCTMNESKSTSLKDFLESLKSIMQ
MDYS
The VH/VL domain linker sequence and the
linker between the VL domain and human IL4
are shown in bold. The sequence of murine IL4
is shown in italics.
Amino acid sequence of the KSF-IL10 conjugate
(SEQ ID NO: 65)
EVQLLESGGGLVQPGGSLRLSCAASGFTFSSYAMSWVRQAPGKGL
EWVSAISGSGGSTYYADSVKGRFTISRDNSKNTLYLQMNSLRAED
TAVYYCAKSPKVSLFDYWGQGTLVTVSSGGSGGSELTQDPAVSVA
LGQTVRITCQGDSLRSYYASWYQQKPGQAPVLVIYGKNNRPSGIP
DRFSGSSSGNTASLTITGAQAEDEADYYCNSSPLNRLAVVFGGGT
KLTVLGSSSSGSSSSGSSSSGSPGQGTOSENSCTHFPGNLPNMLR
DLRDAFSRVKTFFQMKDOLDNLLLKESLLEDFKGYLGCQALSEMI
QFYLEEVMPQAENQDPDIKAHVNSLGENLKTLRLRLRRCHRFLPC
ENKSKAVEQVKNAFNKLQEKGIYKAMSEFDIFINYIEAYMTMKIR
N
The VH/VL domain linker sequence and the
linker between the VL domain and IL10 are
shown in bold. The sequence of IL10 is shown
in italics.
Amino acid sequence of the L19-IL10 conjugate
used in cancer experiments
(SEQ ID NO: 66)
EVQLLESGGGLVQPGGSLRLSCAASGFTFSSFSMSWVRQAPGKGL
EWVSSISGSSGTTYYADSVKGRFTISRDNSKNTLYLQMNSLRAED
TAVYYCAKPFPYFDYWGQGTLVTVSSGGSGGEIVLTQSPGTLSLS
PGERATLSCRASQSVSSSFLAWYQQKPGQAPRLLIYYASSRATGI
PDRFSGSGSGTDFTLTISRLEPEDFAVYYCQQTGRIPPTFGQGTK
VEIKSSSSGSSSSGSSSSGSPGQGTQSENSCTHFPGNLPNMLRDL
RDAFSRVKTFFQMKDQLDNLLLKESLLEDFKGYLGCCIALSEMIQ
FYLEEVMPQAENQDPDIKAHVNSLGENLKTLRLRLRRCHRFLPCE
NKSKAVEQVKNAFNKLQEKGIYKAMSEFDIFINYIEAYMTMKIRN
The VH/VL domain linker sequence and the
linker between the VL domain and IL10 are
shown in bold. The sequence of IL10 is shown
in italics.
Amino acid sequence of the human IL4 N38Q
mutant
(SEQ ID NO: 67)
HKCDITLQEIIKTLNSLTEQKTLCTELTVTDIFAASKQTTEKETF
CRAATVLRQFYSHHEKDTRCLGATAQQFHRHKQLIRFLKRLDRNL
WGLAGLNSCPVKEANQSTLENFLERLKTIMREKYSKCSS
Amino acid sequence of F8- (human) IL4 N284Q
mutant
(SEQ ID NO: 68)
EVQLLESGGGLVQPGGSLRLSCAASGFTFSLFTMSWVRQAPGKGL
EWVSAISGSGGSTYYADSVKGRFTISRDNSKNTLYLQMNSLRAED
TAVYYCAKSTHLYLFDYWGQGTLVTVSSGGSGGEIVLTQSPGTLS
LSPGERATLSCRASQSVSMPFLAWYQQKPGQAPRLLIYGASSRAT
GIPDRFSGSGSGTDFTLTISRLEPEDFAVYYCQQMRGRPPTFGQG
TKVEIKSSSSGSSSSGSSSSGHKCDITLQEIIKTLNSLTEQKTLC
TELTVTDIFAASKQTTEKETFCRAATVLRQFYSHHEKDTRCLGAT
AQQFHRHKQLIRFLKRLDRNLWGLAGLNSCPVKEANQSTLENFLE
RLKTIMREKYSKCSS
The VH/VL domain linker sequence and the
linker between the VL domain and IL4 are
shown in bold and underlined. The sequence
of the mutant IL4 is shown in italics
Amino acid sequence of F8-SIP
(SEQ ID NO: 69)
EVQLLESGGGLVQPGGSLRLSCAASGFTFSLFTMSWVRQAPGKGL
EWVSAISGSGGSTYYADSVKGRFTISRDNSKNTLYLQMNSLRAED
TAVYYCAKSTHLYLFDYWGQGTLVTVSSGGGGSGGGGSGGGGEIV
LTQSPGTLSLSPGERATLSCRASQSVSMPFLAWYQQKPGQAPRLL
IYGASSRATGIPDRFSGSGSGTDFTLTISRLEPEDFAVYYCQQMR
GRPPTFGQGTKVEIKSGGSGGPRAAPEVYAFATPEWPGSRDKRTL
ACLIQNFMPEDISVQWLHNEVQLPDARHSTTQPRKTKGSGFFVFS
RLEVTRAEWEQKDEFICRAVHEAASPSQTVQRAVSVNPESSRRGG
C
The VH/VL domain linker sequence and the
linker between the VL and epsilon-CH4 domain
are shown in bold the epsilon-CH4 domain of
the human IgE is shown in italics.

### REFERENCES

Borsi L, Balza E, Carnemolla B, Sassi F, Castellani P, Berndt A, at al. Selective targeted delivery of TNFalpha to tumor blood vessels. Blood. 2003; 102:4384-92.
Brack S S, Silacci M, Birchler M, Neri D. Tumor-targeting properties of novel antibodies specific to the large isoform of tenascin-C. Clin Cancer Res. 2006; 12:3200-8.
Carnemolla B, Borsi L, Balza E, Castellani P, Meazza R, Berndt A, et al. Enhancement of the antitumor properties of interleukin-2 by its targeted delivery to the tumor blood vessel extracellular matrix. Blood. 2002; 99:1659-65.
Chomarat P, Banchereau J. An update on interleukin-4 and its receptor. Eur Cytokine Netw. 1997; 8:333-44.
Dela Cruz, J. S., Huang, T. H., Penichet, M. L. & Morrison, S. L. Antibody-cytokine fusion proteins: innovative weapons in the war against cancer. Clin Exp Med 4, 57-64 (2004).
Ebbinghaus et al., Int. J. Cancer, 2005, 116, 304-313.
Eigentler T K, Weide B, de Braud F, Spitaleri G, Romanini A, Pflugfelder A, et al. A dose-escalation and signal-generating study of the immunocytokine L19-IL2 in combination with dacarbazine for the therapy of patients with metastatic melanoma. Clin Cancer Res. 2011; 17:7732-42.
Fort M, Lesley R, Davidson N, Menon S, Brombacher F, Leach M, Rennick D. IL-4 Exacerbates Disease in a Th1 Cell Transfer Model of Colitis. J. Immunol. 2001; 166:2793-2800.
Frey K, Schliemann C, Schwager K, Giavazzi R, Johannsen M, Neri D. The immunocytokine F8-IL2 improves the therapeutic performance of sunitinib in a mouse model of renal cell carcinoma. J Urol. 2010; 184:2540-8.
Frey K, Zivanovic A, Schwager K, Neri D. Antibody-based targeting of interferon-alpha to the tumor neovasculature: a critical evaluation. Integr Biol (Camb). 2011; 3:468-78.
Frey K, Zivanovic A, Schwager K, Neri D., Antibody-based targeting of interferon-alpha to the tumor neovasculature: a critical evaluation. Integr Biol (Camb). 2011 April; 3(4):468-78.
Fukushi J, Ono M, Morikawa W, Iwamoto Y, Kuwano M. The Activity of Soluble VCAM-1 in angiogenesis Stimulated by IL-4 and IL-13. J. Immunol. 2000; 165:2818-2823.
Janeway C. Immunobiology: the immune system in health and disease. 6th ed. New York: Garland Science; 2005.
Joosten Leo A B, Erik Lubberts, Monique M A Helsen, Tore Saxne, Christina J J Coenen-de Roo, Dick Heineg\dot{a}rd and Wim B van den Berg. Protection against cartilage and bone destruction by systemic interleukin-4 treatment in established murine type II collagen induced arthritis, Arthritis Research, Vol 1, No 1, 81-91 (1999).
Joosten, Leo A. B., Erik Lubberts, Patrick Durez, Monique M. A. Helsen, Marike J. M. Jacobs, Michel Goldman, and Wim B. Van Den Berg, Role Of Interleukin-4 And Interleukin-10 In Murine Collagen-Induced Arthritis, Arthritis And Rheumatism, Vol. 40, No. 2, 249-260 (1997).
Kaspar M, Trachsel E, Neri D. The antibody-mediated targeted delivery of interleukin-15 and GM-CSF to the tumor neovasculature inhibits tumor growth and metastasis. Cancer Res. 2007; 67:4940-8.
Kontermann R E. Antibody-cytokine fusion proteins. Arch Biochem Biophys. 2012; 526:194-205.
Kurtz D M, Tschetter L K, Allred J B, Geyer S M, Kurtin P J, Putnam W D, et al. Subcutaneous interleukin-4 (IL-4) for relapsed and resistant non-Hodgkin lymphoma: a phase II trial in the North Central Cancer Treatment Group, NCCTG 91-78-51. Leuk Lymphoma. 2007; 48:1290-8.
Li Z, Chen L, Qin Z. Paradoxical roles of IL-4 in tumor immunity. Cell Mol Immunol. 2009; 6(6):415-422.
Li Z, Jiang J, Wang Z, Zhang J, Xiao M, Wang C, Lu Y, Qin Z. Endogenous interleukin-4 promotes tumor development by increasing tumor cell resistance to apoptosis. Cancer Res. 2008; 68(21):8687-8694.
McCarthy D P, Richards M H, Miller S D. Mouse models of multiple sclerosis: experimental autoimmune encephalomyelitis and Theiler's virus-induced demyelinating disease. Methods Mol Biol. 2012; 900:381-401.
Neri, D. & Bicknell, R. Tumour vascular targeting. Nat Rev Cancer 5, 436-446 (2005).
OuYang Z, Osuga Y, Hirota Y, Hirata T, Yoshino O, Koga K, Yano T, Taketani T. Interleukin-4 induces expression of eotaxin in endometriotic stromal cells. Fertility and Sterility. 2010; 94(1): 58-62.
OuYang Z, Hirota Y, Osuga Y, Hamasaki K, Hasegawa A, Tajima T, Hirata T, Koga K, Yoshino O, Harada M, Takemura Y, Nose E, Yano T, Taketani Y. Interleukin-4 Stimulates Proliferation of Endometriotic Stromal Cells. The American Journal of Pathology. 2008. 173(2) 463-469.
Papadia F, Basso V, Patuzzo R, Maurichi A, Di Florio A, Zardi L, et al. Isolated limb perfusion with the tumor-targeting human monoclonal antibody-cytokine fusion protein L19-TNF plus melphalan and mild hyperthermia in patients with locally advanced extremity melanoma. J Surg Oncol. 2012.
Pasche N, Frey K, Neri D. The targeted delivery of IL17 to the mouse tumor neo-vasculature enhances angiogenesis but does not reduce tumor growth rate. Angiogenesis. 2012; 15:165-9.
Pasche N, Neri D. Immunocytokines: a novel class of potent armed antibodies. Drug Discov Today. 2012; 17:583-90.
Pasche N, Woytschak J, Wulhfard S, Villa A, Frey K, Neri D. Cloning and characterization of novel tumor-targeting immunocytokines based on murine IL7. J Biotechnol. 2011; 154:84-92.
Pasche N, Wulhfard S, Pretto F, Carugati E, Neri D. The antibody-based delivery of interleukin-12 to the tumor neovasculature eradicates murine models of cancer in combination with Paclitaxel. Clin Cancer Res. 2012; 18:4092-103.
Pedretti M, Rancic Z, Soltermann A, Herzog B A, Schliemann C, Lachat M, at al. Comparative immunohistochemical staining of atherosclerotic plaques using F16, F8 and L19: Three clinical-grade fully human antibodies. Atherosclerosis. 2010; 208:382-9.
Pedretti M, Soltermann A, Arni S, Weder W, Neri D, Hillinger S. Comparative immunohistochemistry of L19 and F16 in non-small cell lung cancer and mesothelioma: two human antibodies investigated in clinical trials in patients with cancer. Lung Cancer. 2009; 64:28-33.
Pedretti M, Verpelli C, Marlind J, Bertani G, Sala C, Neri D, et al. Combination of temozolomide with immunocytokine F16-IL2 for the treatment of glioblastoma. Br J Cancer. 2010; 103:827-36.
Pfaffen S, Frey K, Stutz I, Roesli C, Neri D. Tumour-targeting properties of antibodies specific to MMP-1A, MMP-2 and MMP-3, Eur. J. Nucl. Med. Mol. Imaging 37(8):1559-65 (2010)
Reisfeld, R. A., Becker, J. C. & Gillies, S. D. Immunocytokines: a new approach to immunotherapy of melanoma. Melanoma Res 7 Suppl 2, S99-106 (1997).
Rybak J N, Roesli C, Kaspar M, Villa A, Neri D. The extra-domain A of fibronectin is a vascular marker of solid tumors and metastases. Cancer Res. 2007; 67:10948-57.
Sauer S, Erba P A, Petrini M, Menrad A, Giovannoni L, Grana C, et al. Expression of the oncofetal ED-B-containing fibronectin isoform in hematologic tumors enables ED-B-targeted 131I-L19SIP radioimmunotherapy in Hodgkin lymphoma patients. Blood. 2009; 113:2265-74.
Savage, P., So, A., Spooner, R. A. & Epenetos, A. A. A recombinant single chain antibody interleukin-2 fusion protein. Br J Cancer 67, 304-310 (1993).
Schiffmann S, Ferreiros N, Birod K, Eberle M, Schreiber Y, Pfeilschifter W, Ziemann U, Pierre S, Scholich K, Grösch S, Geisslinger G. J. Ceramide synthase 6 plays a critical role in the development of experimental autoimmune encephalomyelitis. J. Immunol., 188(11):5723-33 (2012)
Schliemann C, Palumbo A, Zuberbuhler K, Villa A, Kaspar M, Trachsel E, et al. Complete eradication of human B-cell lymphoma xenografts using rituximab in combination with the immunocytokine L19-IL2. Blood. 2009; 113:2275-83.
Schliemann C, Wiedmer A, Pedretti M, Szczepanowski M, Klapper W, Neri D. Three clinical-stage tumor targeting antibodies reveal differential expression of oncofetal fibronectin and tenascin-C isoforms in human lymphoma. Leuk Res. 2009; 33:1718-22.
Schrama, D., Reisfeld, R. A. & Becker, J. C. Antibody targeted drugs as cancer therapeutics. Nat Rev Drug Discov 5, 147-159 (2006).
Schwager et al., Preclinical characterization of DEKAVIL (F8-IL10), a novel clinical-stage immunocytokine which inhibits the progression of collagen-induced arthritis. Arthritis Research & Therapy 2009, 11(5):R142.
Schwager K, Bootz F, Imesch P, Kaspar M, Trachsel E, Neri D. The antibody-mediated targeted delivery of interleukin-10 inhibits endometriosis in a syngeneic mouse model. Hum Reprod. 2011; 26:2344-52.
Schwager K, Kaspar M, Bootz F, Marcolongo R, Paresce E, Neri D. et al. Preclinical characterization of DEKAVIL (F8-IL10), a novel clinical-stage immunocytokine which inhibits the progression of collagen-induced arthritis. Arthritis Res Ther. 2009; 11:R142.
Schwager K, Villa A, Rosli C, Neri D, Rosli-Khabas M, Moser G. A comparative immunofluorescence analysis of three clinical-stage antibodies in head and neck cancer. Head Neck Oncol. 2011; 3:25.
Somigliana E, Viganò P, Rossi G, Carinelli S, Vignali M, Panina-Bordignon P. Endometrial ability to implant in ectopic sites can be prevented by interleukin-12 in a murine model of endometriosis. Human Reprod., 14(12):2044-2950 (1999).
Trachsel et al., Antibody-mediated delivery of IL-10 inhibits the progression of established collagen-induced arthritis. Arthritis Research & Therapy, 2007, 9(1):R9.
Tepper R I, Pattengale P K, Leder P. Murine interleukin-4 displays potent anti-tumor activity in vivo. Cell. 1989; 57:503-12.
Tepper R I. The tumor-cytokine transplantation assay and the antitumor activity of interleukin-4. Bone Marrow Transplant. 1992; 9 Suppl 1:177-81.
Villa A, Trachsel E, Kaspar M, Schliemann C, Sommavilla R, Rybak J N, at al. A high-affinity human monoclonal antibody specific to the alternatively spliced EDA domain of fibronectin efficiently targets tumor neo-vasculature in vivo. Int J Cancer. 2008; 122:2405-13.
Viti F, Tarli L, Giovannoni L, Zardi L, Neri D. Increased binding affinity and valence of recombinant antibody fragments lead to improved targeting of tumoral angiogenesis. Cancer Res. 1999; 59:347-52.
Volpert O V, Fong T, Koch A E, Peterson J D, Waltenbaugh C, Tepper R I, et al. Inhibition of angiogenesis by interleukin 4. J Exp Med. 1998; 188:1039-46.
Walz M, Overbergh L, Mathieu C, Kolb H, Martin S. A Murine Interleukin-4-Ig Fusion Protein Regulates the Expression of Th1- and Th2-Specific Cytokines in the Pancreas of NOD Mice. Horm. Metab. Res. 2002; 34:561-569.
Wei M X, Tamiya T, Hurford R K, Jr., Boviatsis E J, Tepper R I, Chiocca E A. Enhancement of interleukin-4-mediated tumor regression in athymic mice by in situ retroviral gene transfer. Hum Gene Ther. 1995; 6:437-43.
Whitehead R P, Lew D, Flanigan R C, Weiss G R, Roy V, Glode M L, et al. Phase II trial of recombinant human interleukin-4 in patients with advanced renal cell carcinoma: a southwest oncology group study. J Immunother. 2002; 25:352-8.
Whitehead R P, Unger J M, Goodwin J W, Walker M J, Thompson J A, Flaherty L E, et al. Phase II trial of recombinant human interleukin-4 in patients with disseminated malignant melanoma: a Southwest Oncology Group study. J Immunother. 1998; 21:440-6.
Wiernik P H, Dutcher J P, Yao X, Venkatraj U, Falkson C I, Rowe J M, et al. Phase II study of interleukin-4 in indolent B-cell non-Hodgkin lymphoma and B-cell chronic lymphocytic leukemia: a study of the Eastern Cooperative Oncology Group (E5Y92). J Immunother. 2010; 33:1006-9.
Yu J S, Wei M X, Chiocca E A, Martuza R L, Tepper R I. Treatment of glioma by engineered interleukin 4-secreting cells. Cancer Res. 1993; 53:3125-8.
Zamorano J, Wang H Y, Wang L M, Pierce J H, Keegan A D. IL-4 protects cells from apoptosis via the insulin receptor substrate pathway and a second independent signaling pathway. J Immunol. 1996; 157:4926-34.

1. A conjugate comprising interleukin-4 (IL4) and a specific binding member which binds the Extra Domain-A (ED-A) of fibronectin.

2. A conjugate according to claim 1, wherein the specific binding member is an antibody.

3. A conjugate according to claim 2, wherein the specific binding member is a diabody.

4-5. (canceled)

6. A conjugate according to claim 1, wherein the specific binding member comprises an antigen binding site having the complementarity determining regions (CDRs) of antibody F8 set forth in SEQ ID NOs 12-17.

7. A conjugate according to claim 6, wherein the specific binding member comprises the VH and VL domains of antibody F8 set forth in SEQ ID NOs 18 and 19.

8. A conjugate according to claim 7, wherein the specific binding member comprises the amino acid sequence of antibody F8 set forth in SEQ ID NO: 20.

9. A conjugate according to claim 8, wherein the conjugate comprises the amino acid sequence set forth in SEQ ID NO: 22.

10-17. (canceled)

18. A conjugate according to any one of claim 1, wherein the IL4 is human IL4.

19. A conjugate according to claim 18, wherein the IL4 comprises or consists of the sequence set forth in SEQ ID NO: 54; or comprises or consist of the sequence set forth in SEQ ID NO: 54, except that the residue at position 38 of SEQ ID NO: 54 is a serine, glutamine, or alanine residue rather than an asparagine residue.

20. A conjugate according to claim 19, wherein the IL4 comprises or consist of the sequence set forth in SEQ ID NO: 67.

21-27. (canceled)

28. A method of treating of an inflammatory autoimmune disease in a patient, the method comprising administering a therapeutically effective amount of a conjugate according to claim 1 to the patient.

29. A method of delivering IL4 to sites of inflammatory autoimmune disease in a human or animal comprising administering to the human or animal a conjugate according to claim 1.

30-31. (canceled)

32. The method according to claim 28, wherein the method further comprises administering a glucocorticoid to the individual.

33. The method according to claim 32, wherein the glucocorticoid is dexametehasone.

34. The method according to claim 28, wherein the inflammatory autoimmune disease is rheumatoid arthritis (RA), endometriosis, autoimmune diabetes, inflammatory bowel disease (IBD), psoriasis, psoriatic arthritis, peridontitis, or multiple sclerosis (MS).

35-95. (canceled)

96. The method according to claim 29, wherein the inflammatory autoimmune disease is rheumatoid arthritis (RA), multiple sclerosis (MS), endometriosis, autoimmune diabetes, inflammatory bowel disease (IBD), psoriasis, psoriatic arthritis, or peridontitis.

97. A conjugate according to claim 19, wherein the IL4 comprises or consists of sequence set forth in SEQ ID NO: 54, except that the residue at position 38 of SEQ ID NO: 54 is a glutamine residue rather than an asparagine residue.


SEQUENCE LISTING (69 sequences)

SEQ ID NO: 1 - DNA, 9 bp, Artificial
  Synthetic sequence F8 CDR1 VH of PCT/EP2014/053998
ctgtttacg

SEQ ID NO: 2 - DNA, 18 bp, Artificial
  Synthetic sequence F8 CDR2 VH of PCT/EP2014/053998
agtggtagtggtggtagc

SEQ ID NO: 3 - DNA, 18 bp, Artificial
  Synthetic sequence F8 CDR3 VH of PCT/EP2014/053998
agtactcatttgtatctt

SEQ ID NO: 4 - DNA, 9 bp, Artificial
  Synthetic sequence F8 CDR1 VL of PCT/EP2014/053998
atgccgttt

SEQ ID NO: 5 - DNA, 21 bp, Artificial
  Synthetic sequence F8 CDR2 VL of PCT/EP2014/053998
ggtgcatccagcagggccact

SEQ ID NO: 6 - DNA, 18 bp, Artificial
  Synthetic sequence F8 CDR3 VL of PCT/EP2014/053998
atgcgtggtcggccgccg

SEQ ID NO: 7 - DNA, 354 bp, Artificial
  Synthetic sequence Nucleotide sequence of F8-VH of PCT/EP2014/053998
gaggtgcagctgttggagtctgggggaggcttggtacagcctggggggtccctgagactctcctgtgcagcctctggattcacctttagcctgtttacgatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaagtactcatttgtatctttttgactactggggccagggaaccctggtcaccgtctcgagt

SEQ ID NO: 8 - DNA, 324 bp, Artificial
  Synthetic sequence Nucleotide sequence of F8-VL of PCT/EP2014/053998
gaaattgtgttgacgcagtctccaggcaccctgtctttgtctccaggggaaagagccaccctctcctgcagggccagtcagagtgttagcatgccgtttttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagatgcgtggtcggccgccgacgttcggccaagggaccaaggtggaaatcaaa

SEQ ID NO: 9 - DNA, 693 bp, Artificial sequence
  Synthetic sequence Nucleotide sequence of the F8 diabody of PCT/EP2014/053998
gaggtgcagctgttggagtctgggggaggcttggtacagcctggggggtccctgagactctcctgtgcagcctctggattcacctttagcctgtttacgatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaagtactcatttgtatctttttgactactggggccagggaaccctggtcaccgtctcgagtggcggtagcggaggggaaattgtgttgacgcagtctccaggcaccctgtctttgtctccaggggaaagagccaccctctcctgcagggccagtcagagtgttagcatgccgtttttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagatgcgtggtcggccgccgacgttcggccaagggaccaaggtggaaatcaaa

SEQ ID NO: 10 - DNA, 1098 bp, Artificial sequence
  Synthetic sequence Nucleotide sequence of F8 - (murine) IL4 of PCT/EP2014/053998
gaggtgcagctgttggagtctgggggaggcttggtacagcctggggggtccctgagactctcctgtgcagcctctggattcacctttagcctgtttacgatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaagtactcatttgtatctttttgactactggggccagggaaccctggtcaccgtctcgagtggcggtagcggaggggaaattgtgttgacgcagtctccaggcaccctgtctttgtctccaggggaaagagccaccctctcctgcagggccagtcagagtgttagcatgccgtttttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagatgcgtggtcggccgccgacgttcggccaagggaccaaggtggaaatcaaatcttcctcatcgggtagtagctcttccggctcatcgtccagcggccatatccacggatgcgacaaaaatcacttgagagagatcatcggcattttgaacgaggtcacaggagaagggacgccatgcacggagatggatgtgccaaacgtcctcacagcaacgaagaacaccacagagagtgagctcgtctgtagggcttccaaggtgcttcgcatattttatttaaaacatgggaaaactccatgcttgaagaagaactctagtgttctcatggagctgcagagactctttcgggcttttcgatgcctggattcatcgataagctgcaccatgaatgagtccaagtccacatcactgaaagacttcctggaaagcctaaagagcatcatgcaaatggattactcg

SEQ ID NO: 11 - DNA, 1125 bp, Artificial sequence
  Synthetic sequence Nucleotide sequence of F8 - (human) IL4 of PCT/EP2014/053998
gaggtgcagctgttggagtctgggggaggcttggtacagcctggggggtccctgagactctcctgtgcagcctctggattcacctttagcctgtttacgatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaagtactcatttgtatctttttgactactggggccagggaaccctggtcaccgtctcgagtggcggtagcggaggggaaattgtgttgacgcagtctccaggcaccctgtctttgtctccaggggaaagagccaccctctcctgcagggccagtcagagtgttagcatgccgtttttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagatgcgtggtcggccgccgacgttcggccaagggaccaaggtggaaatcaaatcttcctcatcgggtagtagctcttccggctcatcgtccagcggccacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagc

SEQ ID NO: 12 - protein, 3 aa, Artificial
  Synthetic sequence F8 CDR1 VH of PCT/EP2014/053998
LFT

SEQ ID NO: 13 - protein, 6 aa, Artificial
  Synthetic sequence F8 CDR2 VH of PCT/EP2014/053998
SGSGGS

SEQ ID NO: 14 - protein, 6 aa, Artificial
  Synthetic sequence F8 CDR3 VH of PCT/EP2014/053998
STHLYL

SEQ ID NO: 15 - protein, 3 aa, Artificial
  Synthetic sequence F8 CDR1 VL of PCT/EP2014/053998
MPF

SEQ ID NO: 16 - protein, 7 aa, Artificial
  Synthetic sequence F8 CDR2 VL of PCT/EP2014/053998
GASSRAT

SEQ ID NO: 17 - protein, 6 aa, Artificial
  Synthetic sequence F8 CDR3 VL of PCT/EP2014/053998
MRGRPP

SEQ ID NO: 18 - protein, 118 aa, Artificial
  Synthetic sequence Amino acid sequence of F8 VH of PCT/EP2014/053998
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 19 - protein, 108 aa, Artificial
  Synthetic sequence Amino acid sequence of F8 VL of PCT/EP2014/053998
EIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 20 - protein, 231 aa, Artificial sequence
  Synthetic sequence Amino acid sequence of the F8 diabody of PCT/EP2014/053998
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 21 - protein, 366 aa, Artificial sequence
  Synthetic sequence Amino acid sequence of F8 -(murine) IL4 of PCT/EP2014/053998
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 22 - protein, 375 aa, Artificial sequence
  Synthetic sequence Amino acid sequence of F8 - (human) IL4 of PCT/EP2014/053998
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 23 - protein, 5 aa, Artificial sequence
  Synthetic sequence Amino acid sequence of F8 VH and VL domain diabody linker of PCT/EP2014/053998
GGSGG

SEQ ID NO: 24 - protein, 15 aa, Artificial sequence
  Synthetic sequence Amino acid sequence of F8 VL domain-IL4 linker sequence of PCT/EP2014/053998
SSSSGSSSSGSSSSG

SEQ ID NO: 25 - protein, 5 aa, Artificial
  Synthetic sequence L19 CDR1 VH of PCT/EP2014/053998
SFSMS

SEQ ID NO: 26 - protein, 16 aa, Artificial
  Synthetic sequence L19 CDR2 VH of PCT/EP2014/053998
SISGSSGTTYYADSVK

SEQ ID NO: 27 - protein, 7 aa, Artificial
  Synthetic sequence L19 CDR3 VH of PCT/EP2014/053998
PFPYFDY

SEQ ID NO: 28 - protein, 12 aa, Artificial
  Synthetic sequence L19 CDR1 VL of PCT/EP2014/053998
RASQSVSSSFLA

SEQ ID NO: 29 - protein, 7 aa, Artificial
  Synthetic sequence L19 CDR2 VL of PCT/EP2014/053998
YASSRAT

SEQ ID NO: 30 - protein, 9 aa, Artificial
  Synthetic sequence L19 CDR3 VL of PCT/EP2014/053998
QQTGRIPPT

SEQ ID NO: 31 - protein, 116 aa, Artificial [sequence omitted]

SEQ ID NO: 32 - protein, 108 aa, Artificial [sequence omitted]

SEQ ID NO: 33 - protein, 236 aa, Artificial
  Synthetic sequence Amino acid sequence of scFv(L19) of PCT/EP2014/053998
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 34 - protein, 5 aa, Artificial
  Synthetic sequence F16 CDR1 VH of PCT/EP2014/053998
RYGMS

SEQ ID NO: 35 - protein, 17 aa, Artificial
  Synthetic sequence F16 CDR2 VH of PCT/EP2014/053998
AISGSGGSTYYADSVKG

SEQ ID NO: 36 - protein, 7 aa, Artificial
  Synthetic sequence F16 CDR3 VH of PCT/EP2014/053998
AHNAFDY

SEQ ID NO: 37 - protein, 11 aa, Artificial
  Synthetic sequence F16 CDR1 VL of PCT/EP2014/053998
QGDSLRSYYAS

SEQ ID NO: 38 - protein, 7 aa, Artificial
  Synthetic sequence F16 CDR2 VL of PCT/EP2014/053998
GKNNRPS

SEQ ID NO: 39 - protein, 11 aa, Artificial
  Synthetic sequence F16 CDR3 VL of PCT/EP2014/053998
NSSVYTMPPVV

SEQ ID NO: 40 - protein, 116 aa, Artificial
  Synthetic sequence Amino acid sequence F16 VH domain of PCT/EP2014/053998
EVQLLESGGGLVQPGGSLRLSCAASGFTFSRYGMSWVRQAPGKGLEWVSAISGSGGSTYYADSVKGRFTISRDNSKNTLYLQMNSLRAEDTAVYYCAKAHNAFDYWGQGTLVTVSR

SEQ ID NO: 41 - protein, 108 aa, Artificial
  Synthetic sequence Amino acid sequence F16 VL domain of PCT/EP2014/053998
SSELTQDPAVSVALGQTVRITCQGDSLRSYYASWYQQKPGQAPVLVIYGKNNRPSGIPDRFSGSSSGNTASLTITGAQAEDEADYYCNSSVYTMPPVVFGGGTKLTVL

SEQ ID NO: 42 - protein, 234 aa, Artificial sequence
  Synthetic sequence Amino acid sequence of the scFv(F16) of PCT/EP2014/053998
EVQLLESGGGLVQPGGSLRLSCAASGFTFSRYGMSWVRQAPGKGLEWVSAISGSGGSTYYADSVKGRFTISRDNSKNTLYLQMNSLRAEDTAVYYCAKAHNAFDYWGQGTLVTVSRGGGSGGGSGGSSELTQDPAVSVALGQTVRITCQGDSLRSYYASWYQQKPGQAPVLVIYGKNNRPSGIPDRFSGSSSGNTASLTITGAQAEDEADYYCNSSVYTMPPVVFGGGTKLTVL

SEQ ID NO: 43 - DNA, 1098 bp, Artificial sequence
  Synthetic sequence Nucleotide sequence of KSF-IL4 of PCT/EP2014/053998
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaagacacggccgtatattactgtgcgaaatcgcctaaggtgtcgctttttgactactggggccagggaaccctggtcaccgtctcgagtggcggtagcggagggtctgagctgactcaggaccctgctgtgtctgtggccttgggacagacagtcaggatcacatgccaaggagacagtctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcctctcccctgaatcggctggctgtggtattcggcggagggaccaagctgaccgtcctaggctcttcctcatcgggtagtagctcttccggctcatcgtccagcggccatatccacggatgcgacaaaaatcacttgagagagatcatcggcattttgaacgaggtcacaggagaagggacgccatgcacggagatggatgtgccaaacgtcctcacagcaacgaagaacaccacagagagtgagctcgtctgtagggcttccaaggtgcttcgcatattttatttaaaacatgggaaaactccatgcttgaagaagaactctagtgttctcatggagctgcagagactctttcgggcttttcgatgcctggattcatcgataagctgcaccatgaatgagtccaagtccacatcactgaaagacttcctggaaagcctaaagagcatcatgcaaatggattactcg

SEQ ID NO: 44 - protein, 366 aa, Artificial sequence
  Synthetic sequence Amino acid sequence of KSF-murine IL4 of PCT/EP2014/053998
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPKVSLFDYWGQGTLVTVSSGGSGGSELTQDPAVSVALGQTVRITCQGDSLRSYYASWYQQKPGQAPVLVIYGKNNRPSGIPDRFSGSSSGNTASLTITGAQAEDEADYYCNSSPLNRLAVVFGGGTKLTVLGSSSSGSSSSGSSSSGHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 45 - DNA, 1389 bp, Artificial sequence
  Synthetic sequence Nucleotide sequence of TNFR-Fc of PCT/EP2014/053998
gtgcccgcccaggttgtcttgacaccctacaaaccggaacctgggtacgagtgccagatctcacaggaatactatgacaggaaggctcagatgtgctgtgctaagtgtcctcctggccaatatgtgaaacatttctgcaacaagacctcggacaccgtgtgtgcggactgtgaggcaagcatgtatacccaggtctggaaccagtttcgtacatgtttgagctgcagttcttcctgtaccactgaccaggtggagatccgcgcctgcactaaacagcagaaccgagtgtgtgcttgcgaagctggcaggtactgcgccttgaaaacccattctggcagctgtcgacagtgcatgaggctgagcaagtgcggccctggcttcggagtggccagttcaagagccccaaatggaaatgtgctatgcaaggcctgtgccccagggacgttctctgacaccacatcatccactgatgtgtgcaggccccaccgcatctgtagcatcctggctattcccggaaatgcaagcacagatgcagtctgtgcgcccgagtccccaactctaagtgccatcccaaggacactctacgtatctcagccagagcccacaagatcccaacccctggatcaagagccagggcccagccaaactccaagcatccttacatcgttgggttcaacccccattattgaacaaagtaccaagggtggcgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacaaaaccccgggaggagcagttcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 46 - protein, 463 aa, Artificial sequence
  Synthetic sequence Amino acid sequence of TNFR-Fc of PCT/EP2014/053998
VPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGVPRDCGCKPCICTVPEVSSVFIFPPKPKDVLTITLTPKVTCVVVDISKDDPEVQFSWFVDDVEVHTAQTKPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 47 - DNA, 3012 bp, Artificial sequence
  Synthetic sequence Nucleotide sequence of (murine)IL12-F8-F8 of PCT/EP2014/053998
atgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcgtgcagcaagtgggcatgtgttccctgcagggtccgatccggtggaggcggttcaggcggaggtggctctggcggtggcggatcgagggtcattccagtctctggacctgccaggtgtcttagccagtcccgaaacctgctgaagaccacagatgacatggtgaagacggccagagaaaagcttaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccggtagcgctgatggaggtgaggtgcagctgttggagtctgggggaggcttggtacagcctggggggtccctgagactctcctgtgcagcctctggattcacctttagcctgtttacgatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaagtactcatttgtatctttttgactactggggccagggaaccctggtcaccgtctcgagtggcggtagcggaggggaaattgtgttgacgcagtctccaggcaccctgtctttgtctccaggggaaagagccaccctctcctgcagggccagtcagagtgttagcatgccgtttttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagatgcgtggtcggccgccgacgttcggccaagggaccaaggtggaaatcaaatcttcctcatccggaagtagctcttcgggatcctcgtccagcggcgaggtgcagctgttggagtctgggggaggcttggtacagcctggggggtccctgagactctcctgtgcagcctctggattcacctttagcctgtttacgatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaagtactcatttgtatctttttgactactggggccagggaaccctggtcaccgtctcgagtggcggtagcggaggggaaattgtgttgacgcagtctccaggcaccctgtctttgtctccaggggaaagagccaccctctcctgcagggccagtcagagtgttagcatgccgtttttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagatgcgtggtcggccgccgacgttcggccaagggaccaaggtggaaatcaaa

SEQ ID NO: 48 - protein, 1004 aa, Artificial sequence
  Synthetic sequence Amino acid sequence of (murine)IL12-F8-F8 of PCT/EP2014/053998
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGSADGGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 49 - DNA, 1137 bp, Artificial sequence
  Synthetic sequence Nucleotide sequence of F8-(human)IL2 of PCT/EP2014/053998
gaggtgcagctgttggagtctgggggaggcttggtacagcctggggggtccctgagactctcctgtgcagcctctggattcacctttagcctgtttacgatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaagtactcatttgtatctttttgactactggggccagggaaccctggtcaccgtctcgagtggcggtagcggaggggaaattgtgttgacgcagtctccaggcaccctgtctttgtctccaggggaaagagccaccctctcctgcagggccagtcagagtgttagcatgccgtttttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagatgcgtggtcggccgccgacgttcggccaagggaccaaggtggaaatcaaatcttcctcatcgggtagtagctcttccggctcatcgtccagcggcgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 50 - protein, 379 aa, Artificial sequence
  Synthetic sequence Amino acid sequence of F8-(human)IL2 of PCT/EP2014/053998
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 51 - DNA, 360 bp, Mus musculus
catatccacggatgcgacaaaaatcacttgagagagatcatcggcattttgaacgaggtcacaggagaagggacgccatgcacggagatggatgtgccaaacgtcctcacagcaacgaagaacaccacagagagtgagctcgtctgtagggcttccaaggtgcttcgcatattttatttaaaacatgggaaaactccatgcttgaagaagaactctagtgttctcatggagctgcagagactctttcgggcttttcgatgcctggattcatcgataagctgcaccatgaatgagtccaagtccacatcactgaaagacttcctggaaagcctaaagagcatcatgcaaatggattactcg

SEQ ID NO: 52 - protein, 120 aa, Mus musculus
HIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 53 - DNA, 642 bp, Homo sapiens
tgcatcgttagcttctcctgataaactaattgcctcacattgtcactgcaaatcgacacctattaatgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctgaatattttaatttatgagtttttgatagctttattttttaagtatttatatatttataactcatcataaaataaagtatatatagaatctaaaaaaaaaaaaaaaaaaaaaaaaaa

SEQ ID NO: 54 - protein, 129 aa, Homo sapiens [sequence omitted]

SEQ ID NO: 55 - DNA, 399 bp, Homo sapiens
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 56 - protein, 133 aa, Homo sapiens [sequence omitted]

SEQ ID NO: 57 - DNA, 1554 bp, Artificial sequence
  Synthetic sequence Nucleotide sequence of human IL12 of PCT/EP2014/053998
atatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggaggcggttcaggcggaggtggctctggcggtggcggatcgagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcc

SEQ ID NO: 58 - protein, 518 aa, Artificial sequence
  Synthetic sequence Amino acid sequence of human IL12 of PCT/EP2014/053998
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 59 - protein, 229 aa, Artificial sequence
  Synthetic sequence Amino acid sequence of L19 diabody of PCT/EP2014/053998
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGSSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 60 - protein, 229 aa, Artificial sequence
  Synthetic sequence Amino acid sequence of F16 diabody of PCT/EP2014/053998
EVQLLESGGGLVQPGGSLRLSCAASGFTFSRYGMSWVRQAPGKGLEWVSAISGSGGSTYYADSVKGRFTISRDNSKNTLYLQMNSLRAEDTAVYYCAKAHNAFDYWGQGTLVTVSRGGSGGSSELTQDPAVSVALGQTVRITCQGDSLRSYYASWYQQKPGQAPVLVIYGKNNRPSGIPDRFSGSSSGNTASLTITGAQAEDEADYYCNSSVYTMPPVVFGGGTKLTVL

SEQ ID NO: 61 - protein, 1001 aa, Artificial sequence [sequence omitted]

SEQ ID NO: 62 - protein, 229 aa, Artificial sequence
  Synthetic sequence Amino acid sequence of L19 diabody of PCT/EP2014/053998
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 63 - protein, 404 aa, Artificial sequence
  Synthetic sequence Amino acid sequence of the L19-IL10 conjugate used in the RA experiments of PCT/EP2014/053998
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGSSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKSSSSGSSSSGSSSSGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 64 - protein, 364 aa, Artificial sequence
  Synthetic sequence Amino acid sequence of the L19-murine IL4 conjugate of PCT/EP2014/053998
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKSSSSGSSSSGSSSSGHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 65 - protein, 406 aa, Artificial sequence
  Synthetic sequence Amino acid sequence of the KSF-IL10 conjugate of PCT/EP2014/053998
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPKVSLFDYWGQGTLVTVSSGGSGGSELTQDPAVSVALGQTVRITCQGDSLRSYYASWYQQKPGQAPVLVIYGKNNRPSGIPDRFSGSSSGNTASLTITGAQAEDEADYYCNSSPLNRLAVVFGGGTKLTVLGSSSSGSSSSGSSSSGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 66 - protein, 404 aa, Artificial sequence
  Synthetic sequence Amino acid sequence of the L19-IL10 conjugate used in cancer experiments of PCT/EP2014/053998
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKSSSSGSSSSGSSSSGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 67 - protein, 129 aa, Artificial sequence
  Synthetic sequence Amino acid sequence of the human IL4 N38Q mutant of PCT/EP2014/053998
HKCDITLQEIIKTLNSLTEQKTLCTELTVTDIFAASKQTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 68 - protein, 375 aa, Artificial sequence
  Synthetic sequence Amino acid sequence of F8 - (human) IL4 N284Q mutant of PCT/EP2014/053998
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGHKCDITLQEIIKTLNSLTEQKTLCTELTVTDIFAASKQTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 69 - protein, 361 aa, Artificial sequence
  Synthetic sequence Amino acid sequence of F8-SIP of PCT/EP2014/053998
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGGGSGGGGSGGGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSGGSGGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPESSRRGGC